SEQUENCE LISTING (73 sequences)

SEQ ID NO: 1 - DNA, 614 bp, Bacillus licheniformis
gcatgcgtccttctttgtgcttggaagcagagcccaatattatcccgaaacgataaaacggatgctgaaggaaggaaacgaagtcggcaaccattcctgggaccatccgttattgacaaggctgtcaaacgaaaaagcgtatcaggagattaacgacacgcaagaaatgatcgaaaaaatcagcggacacctgcctgtacacttgcgtcctccatacggcgggatcaatgattccgtccgctcgctttccaatctgaaggtttcattgtgggatgttgatccggaagattggaagtacaaaaataagcaaaagattgtcaatcatgtcatgagccatgcgggagacggaaaaatcgtcttaatgcacgatatttatgcaacgttcgcagatgctgctgaagagattattaaaaagctgaaagcaaaaggctatcaattggtaactgtatctcagcttgaagaagtgaagaagcagagaggctattgaataaatgagtagaagcgccatatcggcgcttttcttttggaagaaaatatagggaaaatggtacttgttaaaaattcggaatatttatacaatatcatatgtatcacattgaaaggggaggagaatc

SEQ ID NO: 2 - DNA, 1251 bp, Streptococcus equisimilis
atgagaacattaaaaaacctcataactgttgtggcctttagtattttttgggtactgttgatttacgtcaatgtttatctctttggtgctaaaggaagcttgtcaatttatggctttttgctgatagcttacctattagtcaaaatgtccttatcctttttttacaagccatttaagggaagggctgggcaatataaggttgcagccattattccctcttataacgaagatgctgagtcattgctagagaccttaaaaagtgttcagcagcaaacctatcccctagcagaaatttatgttgttgacgatggaagtgctgatgagacaggtattaagcgcattgaagactatgtgcgtgacactggtgacctatcaagcaatgtcattgttcaccggtcagaaaaaaatcaaggaaagcgtcatgcacaggcctgggcctttgaaagatcagacgctgatgtctttttgaccgttgactcagatacttatatctaccctgatgctttagaggagttgttaaaaacctttaatgacccaactgtttttgctgcgacgggtcaccttaatgtcagaaatagacaaaccaatctcttaacacgcttgacagatattcgctatgataatgcttttggcgttgaacgagctgcccaatccgttacaggtaatattctcgtttgctcaggcccgcttagcgtttacagacgcgaggtggttgttcctaacatagatagatacatcaaccagaccttcctgggtattcctgtaagtatcggtgatgacaggtgcttgaccaactatgcaactgatttaggaaagactgtttatcaatccactgctaaatgtattacagatgttcctgacaagatgtctacttacttgaagcagcaaaaccgctggaacaagtccttctttagagagtccattatttctgttaagaaaatcatgaacaatccttttgtagccctatggaccatacttgaggtgtctatgtttatgatgcttgtttattctgtggtggatttctttgtagacaatgtcagagaatttgattggctcagggttttggcctttctggtgattatcttcattgttgctctttgtcgtaatattcactatatgcttaagcacccgctgtccttcttgttatctccgttttatggggtactgcatttgtttgtcctacagcccttgaaattgtattctctttttactattagaaatgctgactggggaacacgtaaaaaattatta

SEQ ID NO: 3 - protein, 417 aa, Streptococcus equisimilis
MRTLKNLITVVAFSIFWVLLIYVNVYLFGAKGSLSIYGFLLIAYLLVKMSLSFFYKPFKGRAGQYKVAAIIPSYNEDAESLLETLKSVQQQTYPLAEIYVVDDGSADETGIKRIEDYVRDTGDLSSNVIVHRSEKNQGKRHAQAWAFERSDADVFLTVDSDTYIYPDALEELLKTFNDPTVFAATGHLNVRNRQTNLLTRLTDIRYDNAFGVERAAQSVTGNILVCSGPLSVYRREVVVPNIDRYINQTFLGIPVSIGDDRCLTNYATDLGKTVYQSTAKCITDVPDKMSTYLKQQNRWNKSFFRESIISVKKIMNNPFVALWTILEVSMFMMLVYSVVDFFVDNVREFDWLRVLAFLVIIFIVALCRNIHYMLKHPLSFLLSPFYGVLHLFVLQPLKLYSLFTIRNADWGTRKKLL

SEQ ID NO: 4 - DNA, 1257 bp, Streptococcus pyogenes
gtgcctatttttaaaaaaactttaattgttttatcctttatttttttgatatctatcttgatttatctaaatatgtatctatttggaacatcaactgtaggaatttatggagtaatattaataacctatctagttattaaacttggattatctttcctttatgagccatttaaaggaaagccacatgactataaagttgctgctgtaattccttcttataatgaagatgccgagtcattattagaaactcttaaaagtgtgttagcacagacctatccgttatcagaaatttatattgttgatgatgggagttcaaacacagatgcaatacaattaattgaagagtatgtaaatagagaagtggatatttgtcgaaacgttatcgttcaccgttcccttgtcaataaaggaaaacgccatgctcaagcgtgggcatttgaaagatctgacgctgacgtttttttaaccgtagattcagatacttatatctatccaaatgccttagaagaactcctaaaaagcttcaatgatgagacagtttatgctgcaacaggacatttgaatgctagaaacagacaaactaatctattaacgcgacttacagatatccgttacgataatgcctttggggtggagcgtgctgctcaatcattaacaggtaatattttagtttgctcaggaccattgagtatttatcgacgtgaagtgattattcctaacttagagcgctataaaaatcaaacattcctaggtttacctgttagcattggggatgatcgatgtttaacaaattatgctattgatttaggacgcactgtctaccaatcaacagctagatgtgatactgatgtacctttccaattaaaaagttatttaaagcaacaaaatcgatggaataaatctttttttaaagaatctattatttctgttaaaaaaattctttctaatcccatcgttgccttatggactattttcgaagtcgttatgtttatgatgttgattgtcgcaattgggaatcttttgtttaatcaagctattcaattagaccttattaaactttttgcctttttatccatcatctttatcgttgctttatgtcgtaatgttcattatatgatcaaacatcctgctagttttttgttatctcctctgtatggaatattacacttgtttgtcttacagcccctaaaactttattctttatgcaccattaaaaatacggaatggggaacacgtaaaaaggtcactatttttaaa

SEQ ID NO: 5 - protein, 419 aa, Streptococcus pyogenes
VPIFKKTLIVLSFIFLISILIYLNMYLFGTSTVGIYGVILITYLVIKLGLSFLYEPFKGKPHDYKVAAVIPSYNEDAESLLETLKSVLAQTYPLSEIYIVDDGSSNTDAIQLIEEYVNREVDICRNVIVHRSLVNKGKRHAQAWAFERSDADVFLTVDSDTYIYPNALEELLKSFNDETVYAATGHLNARNRQTNLLTRLTDIRYDNAFGVERAAQSLTGNILVCSGPLSIYRREVIIPNLERYKNQTFLGLPVSIGDDRCLTNYAIDLGRTVYQSTARCDTDVPFQLKSYLKQQNRWNKSFFKESIISVKKILSNPIVALWTIFEVVMFMMLIVAIGNLLFNQAIQLDLIKLFAFLSIIFIVALCRNVHYMIKHPASFLLSPLYGILHLFVLQPLKLYSLCTIKNTEWGTRKKVTIFK

SEQ ID NO: 6 - DNA, 1251 bp, Streptococcus uberis
atggaaaaactaaaaaatctcattacatttatgacttttattttcctgtggctcataattattgggcttaatgtttttgtatttggaactaaaggaagtctaacagtgtatgggattattctattaacctatttgtcgataaaaatgggattatcttttttttatcgtccctataaaggaagtgtaggtcaatataaggtagcagctattatcccatcttataatgaggatggtgtcggtttactagaaactctaaagagtgttcaaaaacaaacatatccaattgcagaaattttcgtaattgacgatgggtcagtagataaaacaggtataaaattggtcgaagactatgtgaagttaaatggctttggagaccaagttatcgttcatcagatgcctgaaaatgttggtaaaagacatgctcaggcttgggcatttgaaaggtctgatgctgatgttttcttaacagtggattcagatacctacatctatcctgatgctcttgaagaattattaaagacatttaatgatccagaggtctacgctgcaactggtcatttaaatgcaagaaatagacaaactaatctcttaactagactgactgatattcgttacgataatgcatttggtgtagaacgtgctgctcagtctgttacgggaaatattttggtttgttccggacctttaagtatttatagacgttccgtcggtattccaaatcttgaacgctatacctcacaaacatttcttggtgtccctgtaagcataggggatgaccgttgtttgacaaattatgcaactgatttgggaaaaacggtttatcagtcaactgcaagatgtgatactgacgttccagataagtttaaggttttcatcaaacaacaaaatcgttggaataagtcattttttagggagtctattatctctgttaagaagttattagccacaccaagtgttgctgtttggactattacagaagtttccatgttcatcatgctagtttattctatctttagcttattgataggagaggctcaagaatttaatctcataaaactggttgcttttttagttattattttcatagtagctctttgtagaaatgttcattacatggttaagcatccatttgcttttttattgtcaccgttttatggattgatacatctattcgttttgcaacctcttaagatatattcgttatttactataagaaatgctacatggggaactcgtaaaaagacaagtaaa

SEQ ID NO: 7 - protein, 416 aa, Streptococcus uberis
MEKLKNLITFMTFIFLWLIIIGLNVFVFGTKGSLTVYGIILLTYLSIKMGLSFFYRPYKGSVGQYKVAAIIPSYNEDGVGLLETLKSVQKQTYPIAEIFVIDDGSVDKTGIKLVEDYVKLNGFGDQVIVHQMPENVGKRHAQAWAFERSDADVFLTVDSDTYIYPDALEELLKTFNDPEVYAATGHLNARNRQTNLLTRLTDIRYDNAFGVERAAQSVTGNILVCSGPLSIYRRSVGIPNLERYTSQTFLGVPVSIGDDRCLTNYATDLGKTVYQSTARCDTDVPDKFKVFIKQQNRWNKSFFRESIISVKKLLATPSVAVWTITEVSMFIMLVYSIFSLLIGEAQEFNLIKLVAFLVIIFIVALCRNVHYMVKHPFAFLLSPFYGLIHLFVLQPLKIYSLFTIRNATWGTRKKTS

SEQ ID NO: 8 - DNA, 2916 bp, Pasteurella multocida
atgaatacattatcacaagcaataaaagcatataacagcaatgactatcaattagcactcaaattatttgaaaagtcggcggaaatctatggacggaaaattgttgaatttcaaattaccaaatgccaagaaaaactctcagcacatccttctgttaattcagcacatctttctgtaaataaagaagaaaaagtcaatgtttgcgatagtccgttagatattgcaacacaactgttactttccaacgtaaaaaaattagtactttctgactcggaaaaaaacacgttaaaaaataaatggaaattgctcactgagaagaaatctgaaaatgcggaggtaagagcggtcgcccttgtaccaaaagattttcccaaagatctggttttagcgcctttacctgatcatgttaatgattttacatggtacaaaaagcgaaagaaaagacttggcataaaacctgaacatcaacatgttggtctttctattatcgttacaacattcaatcgaccagcaattttatcgattacattagcctgtttagtaaaccaaaaaacacattacccgtttgaagttatcgtgacagatgatggtagtcaggaagatctatcaccgatcattcgccaatatgaaaataaattggatattcgctacgtcagacaaaaagataacggttttcaagccagtgccgctcggaatatgggattacgcttagcaaaatatgactttattggcttactcgactgtgatatggcgccaaatccattatgggttcattcttatgttgcagagctattagaagatgatgatttaacaatcattggtccaagaaaatacatcgatacacaacatattgacccaaaagacttcttaaataacgcgagtttgcttgaatcattaccagaagtgaaaaccaataatagtgttgccgcaaaaggggaaggaacagtttctctggattggcgcttagaacaattcgaaaaaacagaaaatctccgcttatccgattcgcctttccgtttttttgcggcgggtaatgttgctttcgctaaaaaatggctaaataaatccggtttctttgatgaggaatttaatcactggggtggagaagatgtggaatttggatatcgcttattccgttacggtagtttctttaaaactattgatggcattatggcctaccatcaagagccaccaggtaaagaaaatgaaaccgatcgtgaagcgggaaaaaatattacgctcgatattatgagagaaaaggtcccttatatctatagaaaacttttaccaatagaagattcgcatatcaatagagtacctttagtttcaatttatatcccagcttataactgtgcaaactatattcaacgttgcgtagatagtgcactgaatcagactgttgttgatctcgaggtttgtatttgtaacgatggttcaacagataataccttagaagtgatcaataagctttatggtaataatcctagggtacgcatcatgtctaaaccaaatggcggaatagcctcagcatcaaatgcagccgtttcttttgctaaaggttattacattgggcagttagattcagatgattatcttgagcctgatgcagttgaactgtgtttaaaagaatttttaaaagataaaacgctagcttgtgtttataccactaatagaaacgtcaatccggatggtagcttaatcgctaatggttacaattggccagaattttcacgagaaaaactcacaacggctatgattgctcaccactttagaatgttcacgattagagcttggcatttaactgatggattcaatgaaaaaattgaaaatgccgtagactatgacatgttcctcaaactcagtgaagttggaaaatttaaacatcttaataaaatctgctataaccgtgtattacatggtgataacacatcaattaagaaacttggcattcaaaagaaaaaccattttgttgtagtcaatcagtcattaaatagacaaggcataacttattataattatgacgaatttgatgatttagatgaaagtagaaagtatattttcaataaaaccgctgaatatcaagaagagattgatatcttaaaagatattaaaatcatccagaataaagatgccaaaatcgcagtcagtattttttatcccaatacattaaacggcttagtgaaaaaactaaacaatattattgaatataataaaaatatattcgttattgttctacatgttgataagaatcatcttacaccagatatcaaaaaagaaatactagccttctatcataaacatcaagtgaatattttactaaataatgatatctcatattacacgagtaatagattaataaaaactgaggcgcatttaagtaatattaataaattaagtcagttaaatctaaattgtgaatacatcatttttgataatcatgacagcctattcgttaaaaatgacagctatgcttatatgaaaaaatatgatgtcggcatgaatttctcagcattaacacatgattggatcgagaaaatcaatgcgcatccaccatttaaaaagctcattaaaacttattttaatgacaatgacttaaaaagtatgaatgtgaaaggggcatcacaaggtatgtttatgacgtatgcgctagcgcatgagcttctgacgattattaaagaagtcatcacatcttgccagtcaattgatagtgtgccagaatataacactgaggatatttggttccaatttgcacttttaatcttagaaaagaaaaccggccatgtatttaataaaacatcgaccctgacttatatgccttgggaacgaaaattacaatggacaaatgaacaaattgaaagtgcaaaaagaggagaaaatatacctgttaacaagttcattattaatagtataactcta

SEQ ID NO: 9 - protein, 972 aa, Pasteurella multocida
MNTLSQAIKAYNSNDYQLALKLFEKSAEIYGRKIVEFQITKCQEKLSAHPSVNSAHLSVNKEEKVNVCDSPLDIATQLLLSNVKKLVLSDSEKNTLKNKWKLLTEKKSENAEVRAVALVPKDFPKDLVLAPLPDHVNDFTWYKKRKKRLGIKPEHQHVGLSIIVTTFNRPAILSITLACLVNQKTHYPFEVIVTDDGSQEDLSPIIRQYENKLDIRYVRQKDNGFQASAARNMGLRLAKYDFIGLLDCDMAPNPLWVHSYVAELLEDDDLTIIGPRKYIDTQHIDPKDFLNNASLLESLPEVKTNNSVAAKGEGTVSLDWRLEQFEKTENLRLSDSPFRFFAAGNVAFAKKWLNKSGFFDEEFNHWGGEDVEFGYRLFRYGSFFKTIDGIMAYHQEPPGKENETDREAGKNITLDIMREKVPYIYRKLLPIEDSHINRVPLVSIYIPAYNCANYIQRCVDSALNQTVVDLEVCICNDGSTDNTLEVINKLYGNNPRVRIMSKPNGGIASASNAAVSFAKGYYIGQLDSDDYLEPDAVELCLKEFLKDKTLACVYTTNRNVNPDGSLIANGYNWPEFSREKLTTAMIAHHFRMFTIRAWHLTDGFNEKIENAVDYDMFLKLSEVGKFKHLNKICYNRVLHGDNTSIKKLGIQKKNHFVVVNQSLNRQGITYYNYDEFDDLDESRKYIFNKTAEYQEEIDILKDIKIIQNKDAKIAVSIFYPNTLNGLVKKLNNIIEYNKNIFVIVLHVDKNHLTPDIKKEILAFYHKHQVNILLNNDISYYTSNRLIKTEAHLSNINKLSQLNLNCEYIIFDNHDSLFVKNDSYAYMKKYDVGMNFSALTHDWIEKINAHPPFKKLIKTYFNDNDLKSMNVKGASQGMFMTYALAHELLTIIKEVITSCQSIDSVPEYNTEDIWFQFALLILEKKTGHVFNKTSTLTYMPWERKLQWTNEQIESAKRGENIPVNKFIINSITL

SEQ ID NO: 10 - DNA, 1200 bp, Bacillus anthracis
taaggttccaaatatgaaagtggcagtagtggtcccctcatataatgaaagtgcaagtgctattgttaatacaattaatagcgttttagctcaagattatccaattcatgaaattttctttgttgatgatggtagtaaggataaatcggcttatgaagtagcacttaaaatgagggaggaacttcttagaactcaacgagaaattgctgctacaactaagaatatttgttctgaaatattaggtattcctgacttaatcgtacatcgtttacctaagaattgcgggaaaagacatgctcaattatgggcttttaaacggacaacagcagatgctattgttaccattgattcagatggtgatttgttcccaaatgctgttagagagttattgaaaccctttaatgatgaaaaagtaatggccacaactggtcacgtgaacattcgtaatagaaatgataatttattaacaaaactaattgatatgcgttatgacaatgcgttccgtgtggagcgtgcagcacagtccgtaacaggaaatgttcttgtttgtagtgggccgttaagttgttatcgtagagaagtaataactgaaaatttagaacattatggaagtcagatgttccttggtgaggaggtgcagtttggagatgatagatgtctaactaattatgctattttgaaagggaaaacagtttatcaatccactgctcgatgtattactgatgctccaactacattaaaacaatttcttaaacagcaactacgttggaacaagtcattttttagagaaagtttaatttcacttggcattggtatgaaaaaaccaaatgttcttgtttggacaattttcgaaatatcgttatggattttatttgggctttccctacttctaagtattattctcaaggcaagtcatgtagggttaattttggctgtttattatttgggttatatttcattagctgtatatgctagaaatgtattttatctattaaaacatccccttactttcttactggcgccattatatggaattctccatgtattagcactattacctatacgcttttatgctttactaactattaaatctaatggttggggaacacgttaattacagtaattttatgtatttttttaggaggatattattaagtgaagattagaaaagcgattatcccagcagcgggattaggcaca

SEQ ID NO: 11 - protein, 366 aa, Bacillus anthracis
MKVAVVVPSYNESASAIVNTINSVLAQDYPIHEIFFVDDGSKDKSAYEVALKMREELLRTQREIAATTKNICSEILGIPDLIVHRLPKNCGKRHAQLWAFKRTTADAIVTIDSDGDLFPNAVRELLKPFNDEKVMATTGHVNIRNRNDNLLTKLIDMRYDNAFRVERAAQSVTGNVLVCSGPLSCYRREVITENLEHYGSQMFLGEEVQFGDDRCLTNYAILKGKTVYQSTARCITDAPTTLKQFLKQQLRWNKSFFRESLISLGIGMKKPNVLVWTIFEISLWILFGLSLLLSIILKASHVGLILAVYYLGYISLAVYARNVFYLLKHPLTFLLAPLYGILHVLALLPIRFYALLTIKSNGWGTR

SEQ ID NO: 12 - DNA, 1380 bp, Sulfolobus solfataricus
tgccctcatgcggttaagcttgaaatggccttttttagaaagagaaaggagttatctagttaaccaatcttgccaccagaaagttatcatagcatatagggaagcaatatattgcactgctagtgcgatagaaccgtatattagagttttcaatttatcttctggaattaaataaatcatggtcattacaaagggtattatagctataaatcctccgtaatgcaagaataaataaaagataaagaagtcggaatggtaacgggttggcagatacaatagtaataagagcttagtattaattacattaattgaggaatagatcttaagaattctagtgaaactcatataaaggaacaagagcgtaaataatggtaacagattggtgtatatcatattaaaaacgtataatgaaccccttttacttatactaccgtcagctatctcccttataaaattaagataatttgctcttgtccatctagttacttgtttcgtaaacatttttatgtctctagggggttttgtatatgccactgcatcaaagacttttacagccctataccctttttttataacaaaatcggttaaatctctatcatcggaaattttaattggtcttccaaacattttcggctctaaaaactctttagataatatatatggttttacgagttcggtcctatatattacacattgtccacttaatattatagcacttccaaaatagtttaccgccctgtttactatctcacttattctctcaaagaattcaccataataatatgcatatttatttttctcgtcatacataattctaatatttggccctactccacctactgactcatcaaaaacacttaacatctttagtatagagtctttataaataatcgtatcactatctagaaacatcactagaggagatcttacatacttaactccctcggctaacgcgtatcttttccccttatgttcacgcatataaataaatttaccaccatatctttccgtaattgatttgtatggttctagaacactatcccctacaacaataaattctaaccttgtgtcatataaagtccttatcactttttcaaaaatatctatttcctccttataaactggtatcacaactgtaagatcagagagattataaaaacttgagtgttgagtttttctattattacttattactgcaaaaaatgaattcaaaaagaaataaagaatagttataattgtgaatgaaagagaataaatgaaatatgagactccgtgaaataagtgaaacataatcaccactataatgctcgatatcgaaatatataacgatttttcctaattcaccattcgaattctccgttcaaaaaggggttagttaac

SEQ ID NO: 13 - protein, 415 aa, Sulfolobus solfataricus
MVIMFHLFHGVSYFIYSLSFTIITILYFFLNSFFAVISNNRKTQHSSFYNLSDLTVVIPVYKEEIDIFEKVIRTLYDTRLEFIVVGDSVLEPYKSITERYGGKFIYMREHKGKRYALAEGVKYVRSPLVMFLDSDTIIYKDSILKMLSVFDESVGGVGPNIRIMYDEKNKYAYYYGEFFERISEIVNRAVNYFGSAIILSGQCVIYRTELVKPYILSKEFLEPKMFGRPIKISDDRDLTDFVIKKGYRAVKVFDAVAYTKPPRDIKMFTKQVTRWTRANYLNFIREIADGSISKRGSLYVFNMIYTNLLPLFTLLFLYMSFTRILKIYSSINVINTKLLLLLYLPTRYHSDFFIFYLFLHYGGFIAIIPFVMTMIYLIPEDKLKTLIYGSIALAVQYIASLYAMITFWWQDWLTR

SEQ ID NO: 14 - DNA, 1680 bp, Ectocarpus siliculosus
gccatttacatcttggcgcgatcgttgcaaagtaaaaataatgttttctgtgacggaaacaggggcatgacatgcttctcggcctgaagaaaaacctcttgttcattgcgtatctgttcttcaatgcccttttaacgcttttgttgcttggattcgattacggctacatcatcgtatcgatattcgttgtcggcggacacttcagagatgtgatcaacgtagcataccaattacttcacatgcacaggatactcaggcgttgtgccgacatcccggaagacgatgccaagatagttatttgttgcttggttccggtgtacaacgagaagccttcgatgttaaagaagaatcttgatgctctgacgacacagaagctatcggaaaacaccaagttggtggtgatgcttctctttgacggactgaacaaccacaacgcagatctcttcaatgccgtcgtcgatgccattggccttgacaccggatgcggagaagagcaatggtttccgaattggaagagcaagctgctgaagaagttggtgtacaaaatcggcatatacaatgacacgtcggtcatcctgtcgtacaaggagaacaattcgggaaaaaaagactctctcatcatcggggaaaacttcatcgtgctcggcatcccgaggatcgaatctctggacgtacgacaagtggatttcatctatcacacggacggcgacaccatttccgacgaaaactgtttgaacgagatggtgaagtctctcgtggatgatccagacctcgacggcgtctctggcctcctgagaacatacctcaaggacgacgcgacttgctcggaaagtgcgttcgtagcgatgcaagactttcagtacttcttctccattgttgtccgtaggatgacggagagcataatgaattcaactacctgcctcccggggtgctccaacatgatcaggataagcgaaaagactcacgctgcgattgaaaaatacggaaaccttccggtcaagaagagcggtctggtgcagacagtcacgcggatgcaaggaaccgaccgacgatacaccacgctcttgctgagacagggttccaagctacagatgaattggcgtgcgtttgttcacacggagccaccgctgaacgcgacggcgtttgtgaatcaacgcagacgatggtcttcaaactccttcttcaattccatgatcacgctgtactccaacaacatcccgatgtacatcaagctatcgaaccttgtcgacatcgcccgagtcttcaccacgatctttcgcgtgatatcgtacttgtgcttttgggtttacgtcaagaatttttcccttgtcaacatcgtttttttctctatatttatcgcccttccctacctctatgccttcgcctggatattctgtatcgttccagagtggaagcagatgatagccggtttttttttgaacaaaatcttcacgccttttttatctgtgatcgcggtcacaaagatgttcttcacttcaaccgatttcgcttggggcagtacgcggttgacaccaccggatgcagcgtcttaaactcaaccaacaatcgtctctattaataaacctgattcgtgattttgggttcgattctcctgtttcctttttttcctttttttcctttttttcctttttttcctattttcctttttgattttgtttt

SEQ ID NO: 15 - protein, 493 aa, Ectocarpus siliculosus virus
MLLGLKKNLLFIAYLFFNALLTLLLLGFDYGYIIVSIFVVGGHFRDVINVAYQLLHMHRILRRCADIPEDDAKIVICCLVPVYNEKPSMLKKNLDALTTQKLSENTKLVVMLLFDGLNNHNADLFNAVVDAIGLDTGCGEEQWFPNWKSKLLKKLVYKIGIYNDTSVILSYKENNSGKKDSLIIGENFIVLGIPRIESLDVRQVDFIYHTDGDTISDENCLNEMVKSLVDDPDLDGVSGLLRTYLKDDATCSESAFVAMQDFQYFFSIVVRRMTESIMNSTTCLPGCSNMIRISEKTHAAIEKYGNLPVKKSGLVQTVTRMQGTDRRYTTLLLRQGSKLQMNWRAFVHTEPPLNATAFVNQRRRWSSNSFFNSMITLYSNNIPMYIKLSNLVDIARVFTTIFRVISYLCFWVYVKNFSLVNIVFFSIFIALPYLYAFAWIFCIVPEWKQMIAGFFLNKIFTPFLSVIAVTKMFFTSTDFAWGSTRLTPPDAAS

SEQ ID NO: 16 - DNA, 1740 bp, Paramecium bursaria Chlorella virus 1
aagacttcttgaaagttacaatgggtaaaaatataatcataatggtttcgtggtacaccatcataacttcaaatctaatcgcggttggaggagcctctctaatcttggctccggcaattactgggtatgttctacattggaatattgctctctcgacaatctggggagtatcagcttatggtattttcgtttttgggtttttccttgcacaagttttattttcagaactgaacaggaaacgtcttcgcaagtggatttctctcagacctaagggttggaatgatgttcgtttggctgtgatcattgctggatatcgcgaggatccttatatgttccagaagtgcctcgagtctgtacgtgactctgattatggcaacgttgcccgtctgatttgtgtgattgacggtgatgaggacgatgatatgaggatggctgccgtttacaaggcgatctacaatgataatatcaagaagcccgagtttgttctgtgtgagtcagacgacaaggaaggtgaacgcatcgactctgatttctctcgcgacatttgtgtcctccagcctcatcgtggaaaacgggagtgtctttatactgggtttcaacttgcaaagatggaccccagtgtcaatgctgtcgttctgattgacagcgataccgttctcgagaaggatgctattctggaagttgtatacccacttgcatgcgatcccgagatccaagccgttgcaggtgagtgtaagatttggaacacagacactcttttgagtcttctcgtcgcttggcggtactattctgcgttttgtgtggagaggagtgcccagtcttttttcaggactgttcagtgcgttggggggccactgggtgcctacaagattgatatcattaaggagattaaggacccctggatttcccagcgctttcttggtcagaagtgtacttacggtgacgaccgccggctaaccaacgagatcttgatgcgtggtaaaaaggttgtgttcactccatttgctgttggttggtctgacagtccgaccaatgtgtttcggtacatcgttcagcagacccgctggagtaagtcgtggtgccgcgaaatttggtacaccctcttcgccgcgtggaagcacggtttgtctggaatttggctggcctttgaatgtttgtatcaaattacatacttcttcctcgtgatttacctcttttctcgcctagccgttgaggccgaccctcgcgcccagacagccacggtgattgtgagcaccacggttgcattgattaagtgtgggtatttttcattccgagccaaggatattcgggcgttttactttgtgctttatacatttgtttactttttctgtatgattccggccaggattactgcaatgatgacgctttgggacattggctgggatactcgcggtggaaacgagaagccttccgttggcacccgggtcgctctgtgggcaaagcaatatctcattgcatatatgtggtgggccgcggttgttggcgctggagtttacagcatcgtccataactggatgttcgattggaattctctttcttatcgttttgctttggttggtatttgttcttacattgtttttattgttattgtgctggtggtttatttcaccggcaaaattacgacttggaatttcacgaagcttcagaaggagctaatcgaggatcgcgttctgtacgatgcaactaccaatgctcagtctgtgtgatttttcctgcaag

SEQ ID NO: 17 - protein, 567 aa, Paramecium bursaria Chlorella virus
MGKNIIIMVSWYTIITSNLIAVGGASLILAPAITGYVLHWNIALSTIWGVSAYGIFVFGFFLAQVLFSELNRKRLRKWISLRPKGWNDVRLAVIIAGYREDPYMFQKCLESVRDSDYGNVARLICVIDGDEDDDMRMAAVYKAIYNDNIKKPEFVLCESDDKEGERIDSDFSRDICVLQPHRGKRECLYTGFQLAKMDPSVNAVVLIDSDTVLEKDAILEVVYPLACDPEIQAVAGECKIWNTDTLLSLLVAWRYYSAFCVERSAQSFFRTVQCVGGPLGAYKDIIKEIKDPWISQRFLGQKCTYGDDRRLTNEILMRGKKVVFTPFAVGWSDSPTNVFRYIVQQTRWSKSWCREIWYTLFAAWKHGLSGIWLAFECLYQITYFFLVIYLFSRLAVEADPRAQTATVIVSTTVALIKCGYFSFRAKDIRAFYFVLYTFVYFFCMIPARITAMMTLWDIGWDTRGGNEKPSVGTRVALWAKQYLIAYMWWAAVVGAGVYSIVHNWMFDWNSLSYRFALVGICSYIVFIVIVLVVYFTGKITTWNFTKLQKELIEDRVLYDATTNAQSV

SEQ ID NO: 18 - DNA, 1203 bp, Streptococcus
gtgaaaatttctgtagcaggctcaggatatgtcggcctatccttgagtattttactggcacaacataatgacgtcactgttgttgatattattgatgaaaaggtgagattgatcaatcaaggcatatctccaatcaaggatgctgatattgaggagtatttaaaaaatgcgccgctaaatctcacagcgacccttgatggcgcaagcgcttatagcaatgcagaccttattatcattgctactccgacaaattatgacagcgaacgcaactactttgacacaaggcatgttgaagaggtcattgagcaggtcctagacctaaatgcgtcagcaaccattattatcaaatcaaccataccactaggctttatcaagcatgttagggaaaaataccagacagatcgtattatttttagcccagaatttttaagagaatcaaaagccttatacgataacctttacccaagtcggatcattgtttcttatgaaaaggacgactcaccaagggttattcaggctgctaaagcctttgctggtcttttaaaggaaggagccaaaagcaaggatactccggtcttatttatgggctcacaggaggctgaggcggtcaagctatttgcgaatacctttttggctatgcgggtgtcttactttaatgaattagacacctattccgaaagcaagggtctagatgctcagcgcgtgattgaaggagtctgtcatgatcagcgcattggtaaccattacaataacccttcctttggatatggcggctattgcctgccaaaggacagcaaacagctgttggcaaattatagaggcattccccagtccttgatgtcagcgattgttgagtccaacaagatacgaaaatcctatttagctgaacaaatattagacagagcctctagtcaaaagcaggctggtgtaccattaacgattggcttttaccgcttgattatgaaaagcaactctgataatttccgagaaagcgccattaaagatattattgatatcatcaacgactatggggttaatattgtcatttacgaacccatgcttggcgaggatattggctacagggttgtcaaggacttagagcagttcaaaaacgagtctacaatcattgtgtcaaatcgctttgaggacgacctaggagatgtcattgataaggtttatacgagagatgtctttggaagagac

SEQ ID NO: 19 - protein, 401 aa, Streptococcus
VKISVAGSGYVGLSLSILLAQHNDVTVVDIIDEKVRLINQGISPIKDADIEEYLKNAPLNLTATLDGASAYSNADLIIIATPTNYDSERNYFDTRHVEEVIEQVLDLNASATIIIKSTIPLGFIKHVREKYQTDRIIFSPEFLRESKALYDNLYPSRIIVSYEKDDSPRVIQAAKAFAGLLKEGAKSKDTPVLFMGSQEAEAVKLFANTFLAMRVSYFNELDTYSESKGLDAQRVIEGVCHDQRIGNHYNNPSFGYGGYCLPKDSKQLLANYRGIPQSLMSAIVESNKIRKSYLAEQILDRASSQKQAGVPLTIGFYRLIMKSNSDNFRESAIKDIIDIINDYGVNIVIYEPMLGEDIGYRVVKDLEQFKNESTIIVSNRFEDDLGDVIDKVYTRDVFGRD

SEQ ID NO: 20 - DNA, 1206 bp, Streptococcus
atgaaaatagcagttgctggatcaggatatgttggattatcactaggagttcttttatcacttcaaaacgaagtcactattgttgatattcttccctctaaagttgataagattaataatggcttatcaccaattcaagatgaatatattgaatattacttaaaaagtaagcaattatctattaaagcaactttagatagcaaagcagcttataaagaagcggaactggtcattattgccacacctacaaattacaacagtagaattaattattttgatacacagcatgttgaaacagttatcaaagaggtactaagcgttaatagccatgcaactcttatcatcaaatcaacaattccaataggtttcattactgaaatgagacagaaattccaaactgatcgtattatcttcagccctgaatttttaagagaatctaaagctttatatgacaacttatatccaagccgaattattgtttcttgtgaagaaaacgattctccaaaagtaaaggcagacgcagaaaaatttgcacttttattaaagtctgcagctaaaaaaaataatgtaccagtacttattatgggagcttcagaagctgaagcagtaaaactatttgccaatacttatttagcgttaagggtagcttattttaatgagttagacacttacgcagaatcgagaaaattaaatagtcacatgattattcaaggaatttcttatgatgatcgaataggaatgcattataataacccatcatttggttatggaggttattgtctacctaaagatacgaagcaattattggcaaattacaataatattcctcaaacgctaattgaagctatcgtttcatcaaataatgtgcgcaagtcctatattgctaagcaaattatcaacgtcttagaagagcgggagtccccagtaaaagtagtcggggtttaccgtttaattatgaaaagtaactcagataattttagagaaagtgctatcaaagatgttattgacattcttaaaagtaaagacattaagataattatttatgagccaatgttaaacaaacttgaatctgaagatcaatctgtacttgtaaatgatttagagaatttcaagaaacaagcaaatattatcgtaactaatcgctatgataatgaattacaagatgttaaaaataaagtttacagtagagatatttttaatagagac

SEQ ID NO: 21 - protein, 402 aa, Streptococcus
MKIAVAGSGYVGLSLGVLLSLQNEVTIVDILPSKVDKINNGLSPIQDEYIEYYLKSKQLSIKATLDSKAAYKEAELVIIATPTNYNSRINYFDTQHVETVIKEVLSVNSHATLIIKSTIPIGFITEMRQKFQTDRIIFSPEFLRESKALYDNLYPSRIIVSCEENDSPKVKADAEKFALLLKSAAKKNNVPVLIMGASEAEAVKLFANTYLALRVAYFNELDTYAESRKLNSHMIIQGISYDDRIGMHYNNPSFGYGGYCLPKDTKQLLANYNNIPQTLIEAIVSSNNVRKSYIAKQIINVLEERESPVKVVGVYRLIMKSNSDNFRESAIKDVIDILKSKDIKIIIYEPMLNKLESEDQSVLVNDLENFKKQANIIVTNRYDNELQDVKNKVYSRDIFNRD

SEQ ID NO: 22 - DNA, 1203 bp, Streptococcus
gtgaaaattgcagttgcaggttctggctatgttggcctatcattaagtgtattattagcacagaaaaatcctgttacagttgtagatattattgagaagaaagtaaatctcataaatcaaaaacaatcaccaatccaggatgttgatattgaaaactatttaaaagaaaaaaagttacaattaagagctactctagacgccgatcaagcatttagggatgcagatatactaattattgctacaccaaccaattatgatgtggagaagaatttttttgatactagtcatgttgagactgtaattgagaaagctttagctttaaatagtcaggctttgttagttattaaatcaacgataccacttggttttattaaaaagatgcgtcaaaaatatcagacagaccgtattatttttagtcccgaatttcttagagagtctaaagctttaaaagataatctttatcctagtcgaataattgtttcctttgaagatgatgattctatggaagtaatagaagcagcaaagacttttgctcaattgttaaaagatggttctttggataaagatgttcctgtactttttatgggttcagcagaggctgaagcagtaaaattatttgccaatacctatttagctatgcgtgtctcctattttaatgagttagatacatatgctgaaaagaatggtttacgtgtggataatattattgagggcgtttgccatgatcgacgcataggaattcattataataacccttcttttggctatggaggatactgcttacctaaagataccaaacagttgctagcaggctatgatggtattcctcaatcgcttataaaagcaattgttgattctaataaaattcgtaaagagtatatcgcatcacaaattttacaacaattgagtgatattaatgtagatcctaaagatgcaacgattggtatttaccgccttatcatgaaaagtaactctgataatttcagagagagtgcaataaaagatattattgatcatattaagagctatcaaattaatatagtcttgtatgagccaatgatgaatgaagattttgatttaccaatcattgatgatttatctgacttcaaagccatgtcacatattatcgtttcaaatagatatgatttagccttagaagatgttaaagaaaaagtttacaccagagatatttacggtgtggat

SEQ ID NO: 23 - protein, 401 aa, Streptococcus
VKIAVAGSGYVGLSLSVLLAQKNPVTVVDIIEKKVNLINQKQSPIQDVDIENYLKEKKLQLRATLDADQAFRDADILIIATPTNYDVEKNFFDTSHVETVIEKALALNSQALLVIKSTIPLGFIKKMRQKYQTDRIIFSPEFLRESKALKDNLYPSRIIVSFEDDDSMEVIEAAKTFAQLLKDGSLDKDVPVLFMGSAEAEAVKLFANTYLAMRVSYFNELDTYAEKNGLRVDNIIEGVCHDRRIGIHYNNPSFGYGGYCLPKDTKQLLAGYDGIPQSLIKAIVDSNKIRKEYIASQILQQLSDINVDPKDATIGIYRLIMKSNSDNFRESAIKDIIDHIKSYQINIVLYEPMMNEDFDLPIIDDLSDFKAMSHIIVSNRYDLALEDVKEKVYTRDIYGVD

SEQ ID NO: 24 - DNA, 1383 bp, Bacillus subtilis
gtgaaaaaaatagctgtcattggaacaggttatgtaggactcgtatcaggcacttgctttgcggagatcggcaataaagttgtttgctgtgatatcgatgaatcaaaaatcagaagcctgaaaaatggggtaatcccaatctatgaaccagggcttgcagacttagttgaaaaaaatgtgctggatcagcgcctgacctttacgaacgatatcccgtctgccattcgggcctcagatattatttatattgcagtcggaacgcctatgtccaaaacaggtgaagctgatttaacgtacgtcaaagcggcggcgaaaacaatcggtgagcatcttaacggctacaaagtgatcgtaaataaaagcacagtcccggttggaacagggaaactggtgcaatctatcgttcaaaaagcctcaaaggggagatactcatttgatgttgtatctaaccctgaattccttcgggaagggtcagcgattcatgacacgatgaatatggagcgtgccgtgattggttcaacaagtcataaagccgctgccatcattgaggaacttcatcagccattccatgctcctgtcattaaaacaaacctagaaagtgcagaaatgattaaatacgccgcgaatgcatttctggcgacaaagatttcctttatcaacgatatcgcaaacatttgtgagcgagtcggcgcagacgtttcaaaagttgctgatggtgttggtcttgacagccgtatcggcagaaagttccttaaagctggtattggattcggcggttcatgttttccaaaggatacaaccgcgctgcttcaaatcgcaaaatcggcaggctatccattcaagctcatcgaagctgtcattgaaacgaacgaaaagcagcgtgttcatattgtagataaacttttgactgttatgggaagcgtcaaagggagaaccatttcagtcctgggattagccttcaaaccgaatacgaacgatgtgagatccgctccagcgcttgatattatcccaatgctgcagcagctgggcgcccatgtaaaagcatacgatccgattgctattcctgaagcttcagcgatccttggcgaacaggtcgagtattacacagatgtgtatgctgcgatggaagacactgatgcatgcctgattttaacggattggccggaagtgaaagaaatggagcttgtaaaagtgaaaaccctcttaaaacagccagtcatcattgacggcagaaatttattttcacttgaagagatgcaggcagccggatacatttatcactctatcggccgtcccgctgttcggggaacggaaccctctgacaagtattttccgggcttgccgcttgaagaattggctaaagacttgggaagcgtcaattta

SEQ ID NO: 25 - protein, 461 aa, Bacillus subtilis
VKKIAVIGTGYVGLVSGTCFAEIGNKVVCCDIDESKIRSLKNGVIPIYEPGLADLVEKNVLDQRLTFTNDIPSAIRASDIIYIAVGTPMSKTGEADLTYVKAAAKTIGEHLNGYKVIVNKSTVPVGTGKLVQSIVQKASKGRYSFDVVSNPEFLREGSAIHDTMNMERAVIGSTSHKAAAIIEELHQPFHAPVIKTNLESAEMIKYAANAFLATKISFINDIANICERVGADVSKVADGVGLDSRIGRKFLKAGIGFGGSCFPKDTTALLQIAKSAGYPFKLIEAVIETNEKQRVHIVDKLLTVMGSVKGRTISVLGLAFKPNTNDVRSAPALDIIPMLQQLGAHVKAYDPIAIPEASAILGEQVEYYTDVYAAMEDTDACLILTDWPEVKEMELVKVKTLLKQPVIIDGRNLFSLEEMQAAGYIYHSIGRPAVRGTEPSDKYFPGLPLEELAKDLGSVNL

SEQ ID NO: 26 - DNA, 900 bp, Streptococcus
atgacaaaggtcagaaaagccattatcccagccgccggcctaggcactcgcttcctacccgccaccaaggcactggccaaggaaatgctcccaatcgtcgataagccaaccattcaattcatcgtcgaggaagctctaaaggccggtatcgaggagattcttgtcgtcaccggcaaggccaaacgctctattgaagaccactttgactccaacttcgagctcgaatacaatctccaagccaagggcaaaaccgagctgctcaagctcgttgatgagaccactgccatcaacctgcacttcattcgtcagagccaccctagaggactaggggacgctgtcctccaggccaaggcctttgtgggcaatgagccctttgtggtcatgctgggggatgacctcatggatattaccaatcctagtgccaagcccttggccaagcagctcattgaggattatgattgcacacacgcctcaacgattgcagtgatgagggtgccgcatgaggaggtttccaattatggcgtgattgcaccgcaagggaaggctgttaagggcttgtatagtgtggagacctttgttgagaagccaagtccagatgaggcaccgagtgacttagcgattattggtcgatatttgttgacgcctgagatttttgccatattggagaatcaggcgcctggggctggcaatgaggtacagctagccgatgcgattgacaagctcaacaagactcagcgggtttttgcgagggagtttaagggagagcggtatgatgttggggacaagtttggctttatgaagacctcacttgactatgctctcaagcaccctcaggtcaaggacgacctcactgactacattataaagctcagtaagcaactgaacaaggacgttaaaaaa

SEQ ID NO: 27 - protein, 300 aa, Streptococcus
MTKVRKAIIPAAGLGTRFLPATKALAKEMLPIVDKPTIQFIVEEALKAGIEEILVVTGKAKRSIEDHFDSNFELEYNLQAKGKTELLKLVDETTAINLHFIRQSHPRGLGDAVLQAKAFVGNEPFVVMLGDDLMDITNPSAKPLAKQLIEDYDCTHASTIAVMRVPHEEVSNYGVIAPQGKAVKGLYSVETFVEKPSPDEAPSDLAIIGRYLLTPEIFAILENQAPGAGNEVQLADAIDKLNKTQRVFAREFKGERYDVGDKFGFMKTSLDYALKHPQVKDDLTDYIIKLSKQLNKDVKK

SEQ ID NO: 28 - DNA, 912 bp, Streptococcus
atgaccaaagtcagaaaagccattattcctgctgcaggtctaggaacacgttttttacctgctaccaaagctcttgccaaagagatgttgcccatcgttgataaaccaaccatccagtttatcgtcgaagaagcgctaaaatctggcatcgaggaaatccttgtggtgaccggaaaagctaaacgctctatcgaggaccattttgattcaaactttgaattagaatacaacctccaagctaaggggaaaaatgaactgttgaaattagtggatgaaaccactgccattaaccttcattttatccgtcaaagccacccaagagggctgggagatgctgtcttacaagccaaagcctttgtgggcaatgaaccctttgtggtcatgcttggagatgacttaatggacattacaaatgcatccgctaaacctctcaccaaacaactcatggaggactatgacaagacgcatgcatccactatcgctgtgatgaaagttcctcatgaagatgtgtctagctatggggttatcgctcctcaaggcaaggctgtcaagggcctttacagtgtagacacctttgttgaaaaaccacaaccagaagatgcgcctagtgatttggctattattggtcgttacctcctaacccctgaaatttttggtattttggaaagacagacccctggagcaggtaacgaagtgcaactcacagatgctatcgataccctcaataaaactcagcgtgtctttgcacgagaatttaaaggcaatcgttacgatgttggggataaatttggattcatgaaaacatctatcgactatgccttagaacacccacaggtcaaagaggacttgaaaaattacattatcaaactaggaaaagctttggaaaaaagtaaagtaccaacacattcaaag

SEQ ID NO: 29 - protein, 304 aa, Streptococcus
MTKVRKAIIPAAGLGTRFLPATKALAKEMLPIVDKPTIQFIVEEALKSGIEEILVVTGKAKRSIEDHFDSNFELEYNLQAKGKNELLKLVDETTAINLHFIRQSHPRGLGDAVLQAKAFVGNEPFVVMLGDDLMDITNASAKPLTKQLMEDYDKTHASTIAVMKVPHEDVSSYGVIAPQGKAVKGLYSVDTFVEKPQPEDAPSDLAIIGRYLLTPEIFGILERQTPGAGNEVQLTDAIDTLNKTQRVFAREFKGNRYDVGDKFGFMKTSIDYALEHPQVKEDLKNYIIKLGKALEKSKVPTHSK

SEQ ID NO: 30 - DNA, 912 bp, Streptococcus
atgactaaagtaagaaaagccattattccagctgccggacttggcacacgttttttaccagcaacaaaagctctcgctaaggaaatgttgcccatcgttgacaaaccaaccattcaattcatcgtggaagaagctttgcgttctggcattgaagaaatcttggtcgtaacaggaaaatcaaaacgctccattgaagaccattttgattccaactttgaactcgaatataatttgcaagaaaaagggaaaactgaactcttaaaattagttgatgaaaccacttctataaacttgcatttcattcgtcaaagtcatcccaaaggcttaggggatgctgttttacaagcaaaagcttttgtaggaaatgaacccttcattgttatgcttggtgacgatttgatggacattacaaataccaaagctgtcccattaaccaaacaattaatggacgattatgaaacaacacatgcttctacaatagccgtaatgaaagttcctcacgatgacgtatcctcttatggtgtcattgctccaaacggcaaagccttgaatggcttatatagcgtggatacctttgttgaaaaaccaaaacctgaggacgcaccaagtgaccttgctatcattggacgttatctcttaacacctgaaatttttgacattcttgaaaatcaagcaccaggtgccggaaacgaagtccaattaactgatgctatcgataccctcaacaaaacacaacgtgtttttgctcgtgagtttactggcaaacgctacgatgttggagacaagtttggcttcatgaaaacatctatcgattatgccctaaaacaccatcaagtcaaagatgacctaaaagcttatattatcaagttaggtaaagaattagaaaaagcacaagattccaaagaaagcaaa

SEQ ID NO: 31 - protein, 304 aa, Streptococcus
MTKVRKAIIPAAGLGTRFLPATKALAKEMLPIVDKPTIQFIVEEALRSGIEEILVVTGKSKRSIEDHFDSNFELEYNLQEKGKTELLKLVDETTSINLHFIRQSHPKGLGDAVLQAKAFVGNEPFIVMLGDDLMDITNTKAVPLTKQLMDDYETTHASTIAVMKVPHDDVSSYGVIAPNGKALNGLYSVDTFVEKPKPEDAPSDLAIIGRYLLTPEIFDILENQAPGAGNEVQLTDAIDTLNKTQRVFAREFTGKRYDVGDKFGFMKTSIDYALKHHQVKDDLKAYIIKLGKELEKAQDSKESK

SEQ ID NO: 32 - DNA, 876 bp, Bacillus subtilis
atgaaaaaagtacgtaaagccataattccagcagcaggcttaggaacacgttttcttccggctacgaaagcaatgccgaaagaaatgcttcctatcgttgataaacctaccattcaatacataattgaagaagctgttgaagccggtattgaagatattattatcgtaacaggaaaaagcaagcgtgcgattgaggatcattttgattactctcctgagcttgaaagaaacctagaagaaaaaggaaaaactgagctgcttgaaaaagtgaaaaaggcttctaacctggctgacattcactatatccgccaaaaagaacctaaaggtctcggacatgctgtctggtgcgcacgcaactttatcggcgatgagccgtttgcggtactgcttggtgacgatattgttcaggctgaaactccagggttgcgccaattaatggatgaatatgaaaaaacactttcttctattatcggtgttcagcaggtgcccgaagaagaaacacaccgctacggcattattgacccgctgacaagtgaaggccgccgttatcaggtgaaaaacttcgttgaaaaaccgcctaaaggcacagcaccttctaatcttgccatcttaggccgttacgtattcacgcctgagatcttcatgtatttagaagagcagcaggttggcgccggcggagaaattcagctcacagacgccattcaaaagctgaatgaaattcaaagagtgtttgcttacgattttgaaggcaagcgttatgatgttggtgaaaagctcggctttatcacaacaactcttgaatttgcgatgcaggataaagagcttcgcgatcagctcgttccatttatggaaggtttactaaacaaagaagaaatc

SEQ ID NO: 33 - protein, 292 aa, Bacillus subtilis
MKKVRKAIIPAAGLGTRFLPATKAMPKEMLPIVDKPTIQYIIEEAVEAGIEDIIIVTGKSKRAIEDHFDYSPELERNLEEKGKTELLEKVKKASNLADIHYIRQKEPKGLGHAVWCARNFIGDEPFAVLLGDDIVQAETPGLRQLMDEYEKTLSSIIGVQQVPEEETHRYGIIDPLTSEGRRYQVKNFVEKPPKGTAPSNLAILGRYVFTPEIFMYLEEQQVGAGGEIQLTDAIQKLNEIQRVFAYDFEGKRYDVGEKLGFITTTLEFAMQDKELRDQLVPFMEGLLNKEEI

SEQ ID NO: 34 - DNA, 1380 bp, Streptococcus
atgaaaaactacgccattatcctagcagctggaaagggaacccgcatgaattcagggctttccaaggtgctgcacaaggtatcaggcctaagcatgctggagcatgtcctcaagagcgtctcagccctagctcctcaaaagcaactcacagtgatcggtcatcaggcagagcaagtacgtgccgtcctaggtgatcaattactgacagtggtgcaagaggagcagctaggaacaggccatgcagtcatgatggcagaagaggagctatctggcttagaagggcagaccctagtgattgcaggtgacacccccttgatcagaggagaaagcctcaaggctctgctagactatcatatcagagaaaagaatgtggcaaccattctcacagccaatgccaaggatccctttggctacggccgaatcattcgcaatgcagcaggagaggtggtcaacatcgttgaacaaaaggacgctaatgaggcagagcaagaggtcaaggagatcaacacagggacctatatctttgacaataagcgcctctttgaggctctaaagcatctcacgactgataatgcccaaggggaatattacctaaccgatgtgatcagtattttcaaggccagccaagaaaaggttggagcttacctgctgaaggattttgatgaaagcctaggggttaatgatcgcctagctctagcccaggctgaggtgatcatgcaggagcggatcaacaagcagcacatgcttaatggggtgaccctgcaaaaccctgcagctacctatatcgaaagcagtgtagagattgcgccggacgtcttgattgaagctaatgtgaccctaaagggacagactagaattggcagcagaagtgttataaccaatgggagctatatccttgattcaaggcttggtgagggcgtagtggtgagccagtcagtgattgagggctcagtcctagcagatggtgtgacagtagggccctatgcacacattcgcccggactctcagctcgatgagtgtgttcatattgggaactttgtagaggttaaggggtctcatctaggggccaataccaaggcagggcatttgacttatctggggaatgccgagattggctcagaggttaatattggtgcaggaagcattacggttaattatgatggtcaacggaaataccagacagtgattggcgatcacgcttttattgggagtcattcgactttgatagctccggtagaggttggggagaatgctttaacagcagcagggtctacgatagcccagtcggtgccagcagacagtgtggctatagggcgtagccgtcaggtggtgaaggaaggctatgccaagaggctaccacatcacccggatcagccccag

SEQ ID NO: 35 - protein, 460 aa, Streptococcus
MKNYAIILAAGKGTRMNSGLSKVLHKVSGLSMLEHVLKSVSALAPQKQLTVIGHQAEQVRAVLGDQLLTVVQEEQLGTGHAVMMAEEELSGLEGQTLVIAGDTPLIRGESLKALLDYHIREKNVATILTANAKDPFGYGRIIRNAAGEVVNIVEQKDANEAEQEVKEINTGTYIFDNKRLFEALKHLTTDNAQGEYYLTDVISIFKASQEKVGAYLLKDFDESLGVNDRLALAQAEVIMQERINKQHMLNGVTLQNPAATYIESSVEIAPDVLIEANVTLKGQTRIGSRSVITNGSYILDSRLGEGVVVSQSVIEGSVLADGVTVGPYAHIRPDSQLDECVHIGNFVEVKGSHLGANTKAGHLTYLGNAEIGSEVNIGAGSITVNYDGQRKYQTVIGDHAFIGSHSTLIAPVEVGENALTAAGSTIAQSVPADSVAIGRSRQVVKEGYAKRLPHHPDQPQ

SEQ ID NO: 36 - DNA, 1368 bp, Bacillus subtilis
atggataagcggtttgcagttgttttagcggctggacaaggaacgagaatgaaatcgaagctttataaagtccttcatccagtttgcggtaagcctatggtagagcacgtcgtggacgaagccttaaaattatctttatcaaagcttgtcacgattgtcggacatggtgcggaagaagtgaaaaagcagcttggtgataaaagcgagtacgcgcttcaagcaaaacagcttggcactgctcatgctgtaaaacaggcacagccatttcttgctgacgaaaaaggcgtcacaattgtcatttgcggagatacgccgcttttgacagcagagacgatggaacagatgctgaaagaacatacacaaagagaagcgaaagctacgattttaactgcggttgcagaagatccaactggatacggccgcattattcgcagcgaaaacggagcggttcaaaaaatagttgagcataaggacgcctctgaagaagaacgtcttgtaactgagatcaacaccggtacgtattgttttgacaatgaagcgctatttcgggctattgatcaggtgtctaatgataatgcacaaggcgagtattatttgccggatgtcatagagattcttaaaaatgaaggcgaaactgttgccgcttaccagactggtaatttccaagaaacgctcggagttaatgatagagttgctctttctcaggcagaacaatttatgaaagagcgcattaataaacggcatatgcaaaatggcgtgacgttgattgacccgatgaatacgtatatttctcctgacgctgttatcggaagcgatactgtgatttaccctggaactgtgattaaaggtgaggtgcaaatcggagaagatacgattattggccctcatacggagattatgaatagtgccattggcagccgtacggttattaaacaatcggtagtcaatcacagtaaagtggggaatgatgtaaacataggaccttttgctcacatcagacctgattctgtcatcgggaatgaagtgaagatcgggaattttgtagaaattaaaaagactcaattcggagaccgaagcaaggcatctcatctaagctatgtcggcgatgctgaggtaggcactgatgtaaacctgggctgcggttcaattactgtcaattatgatggaaagaataagtatttgacaaaaattgaagatggcgcgtttatcggctgcaattccaacttggttgcccctgtcacagtcggagaaggcgcttatgtggcggcaggttcaactgttacggaagatgtacctggaaaagcacttgctattgccagagcgagacaagtaaataaagacgattatgtgaaaaatattcataaaaaa

SEQ ID NO: 37 - protein, 456 aa, Bacillus subtilis
MDKRFAVVLAAGQGTRMKSKLYKVLHPVCGKPMVEHVVDEALKLSLSKLVTIVGHGAEEVKKQLGDKSEYALQAKQLGTAHAVKQAQPFLADEKGVTIVICGDTPLLTAETMEQMLKEHTQREAKATILTAVAEDPTGYGRIIRSENGAVQKIVEHKDASEEERLVTEINTGTYCFDNEALFRAIDQVSNDNAQGEYYLPDVIEILKNEGETVAAYQTGNFQETLGVNDRVALSQAEQFMKERINKRHMQNGVTLIDPMNTYISPDAVIGSDTVIYPGTVIKGEVQIGEDTIIGPHTEIMNSAIGSRTVIKQSVVNHSKVGNDVNIGPFAHIRPDSVIGNEVKIGNFVEIKKTQFGDRSKASHLSYVGDAEVGTDVNLGCGSITVNYDGKNKYLTKIEDGAFIGCNSNLVAPVTVGEGAYVAAGSTVTEDVPGKALAIARARQVNKDDYVKNIHKK

SEQ ID NO: 38 - DNA, 1347 bp, Streptococcus
atgtcacatattacatttgattattcaaaggttcttgagcaatttgccggacagcatgaaattgactttttacaaggtcaggtaacagaggctgatcaggcactacgtcagggcactggacctggatcagatttcttgggctggcttgagttacctgaaaactatgacaaagaagaatttgctcgtatccttaaagcagctgagaagattaaggctgacagtgacgttcttgttgtgattggtattggtggctcttaccttggtgctaaggctgcaattgactttttgaacagccattttgccaacctacaaacagcaaaagagcgcaaagcaccacaaattctttatgctggtaactccatctcatcaagctatcttgctgatcttgtggactatgttcaagataaagatttctctgttaacgtgatttctaagtctggtacaacaacagagcctgcaatcgcctttcgtgtctttaaagaattacttgttaaaaagtacggtcaagaagaggccaacaagcgtatctatgcaacgactgataaggtcaagggtgctgttaaggttgaggctgatgcaaatcattgggaaacctttgttgtgccagataatgttggtggccgtttctcagtgctgacagctgtgggcttgctaccaattgcagcatcaggggctgatattaccgcgctgatggaaggagcaaatgcagctcgtaaggacctgtcatcagataaaatctcagaaaacatcgcttaccaatatgctgtggtccgcaatatcctctatcgcaaaggctatgtaactgaaattttggcaaactatgagccatcattgcagtattttagcgaatggtggaagcaactggctggtgagtctgaaggaaaggaccaaaagggtatttacccaacttcagctaatttctcgacagacctgcattctcttggtcaatttatccaagaaggctaccgtaacctctttgagacagtgattcgtgtggacaagccacgtcaaaatgtgattatcccagaaatggctgaggaccttgatggccttggctacctacaaggaaaagacgttgactttgtcaacaaaaaagcaacagatggtgtccttcttgcccatacagatggtggtgtgccaaatatgtttatcacgcttccagagcaagacgaatttacactaggctatacgatctacttctttgagcttgctattgccctttcaggctacctcaacggggtcaatccatttgatcagccaggcgttgaggcttacaagaaaaacatgtttgcccttcttggtaagccaggctttgaagagctaggagcagcgctcaacgcacgcttg

SEQ ID NO: 39 - protein, 449 aa, Streptococcus
MSHITFDYSKVLEQFAGQHEIDFLQGQVTEADQALRQGTGPGSDFLGWLELPENYDKEEFARILKAAEKIKADSDVLVVIGIGGSYLGAKAAIDFLNSHFANLQTAKERKAPQILYAGNSISSSYLADLVDYVQDKDFSVNVISKSGTTTEPAIAFRVFKELLVKKYGQEEANKRIYATTDKVKGAVKVEADANHWETFVVPDNVGGRFSVLTAVGLLPIAASGADITALMEGANAARKDLSSDKISENIAYQYAVVRNILYRKGYVTEILANYEPSLQYFSEWWKQLAGESEGKDQKGIYPTSANFSTDLHSLGQFIQEGYRNLFETVIRVDKPRQNVIIPEMAEDLDGLGYLQGKDVDFVNKKATDGVLLAHTDGGVPNMFITLPEQDEFTLGYTIYFFELAIALSGYLNGVNPFDQPGVEAYKKNMFALLGKPGFEELGAALNARL

SEQ ID NO: 40 - DNA, 5158 bp, Streptococcus equisimilis
tcaatttatggctttttgctgatagcttacctattagtcaaaatgtccttatcctttttttacaagccatttaagggaagggctgggcaatataaggttgcagccattattccctcttataacgaagatgctgagtcattgctagagaccttaaaaagtgttcagcagcaaacctatcccctagcagaaatttatgttgttgacgatggaagtgctgatgagacaggtattaagcgcattgaagactatgtgcgtgacactggtgacctatcaagcaatgtcattgttcatcggtcagagaaaaatcaaggaaagcgtcatgcacaggcctgggcctttgaaagatcagacgctgatgtctttttgaccgttgactcagatacttatatctaccctgatgctttagaggagttgttaaaaacctttaatgacccaactgtttttgctgcgacgggtcaccttaatgtcagaaatagacaaaccaatctcttaacacgcttgacagatattcgctatgataatgcttttggcgttgaacgagctgcccaatccgttacaggtaatatccttgtttgctcaggtccgcttagcgtttacagacgcgaggtggttgttcctaacatagatagatacatcaaccagaccttcctgggtattcctgtaagtattggtgatgacaggtgcttgaccaactatgcaactgatttaggaaagactgtttatcaatccactgctaaatgtattacagatgttcctgacaagatgtctacttacttgaagcagcaaaaccgctggaacaagtccttctttagagagtccattatttctgttaagaaaatcatgaacaatccttttgtagccctatggaccatacttgaggtgtctatgtttatgatgcttgtttattctgtggtggatttctttgtaggcaatgtcagagaatttgattggctcagggttttagcctttctggtgattatcttcattgttgccctgtgtcggaacattcattacatgcttaagcacccgctgtccttcttgttatctccgttttatggggtgctgcatttgtttgtcctacagcccttgaaattatattctctttttactattagaaatgctgactggggaacacgtaaaaaattattataaaccaactagacctaggttctgacaagggagctaagctagggataaacaaagagttttgatccgactcgagcagctcataaacgaaagctatcccacttgtaattgaagctaagagcttttagcttgcagctctataaagacgaaccagaggctgagtgtcagctttggtgtgagggctaggtcattatgatccttcaggtgtggcacctgagctccggcagtagctaactgtactaaggtatcaaaggaaaaaatgaagtgaaaatttctgtagcaggctcaggatatgtcggcctatccttgagtattttactggcacaacataatgacgtcactgttgttgacattattgatgaaaaggtgagattgatcaatcaaggcatatcgccaatcaaggatgctgatattgaggagtatttaaaaaatgcgccgctaaatctcacagcgacgcttgatggcgcaagcgcttatagcaatgcagaccttattatcattgctactccgacaaattatgacagcgaacgcaactactttgacacaaggcatgttgaagaggtcatcgagcaggtcctagacctaaatgcgtcagcaaccattattatcaaatcaaccataccactaggctttatcaagcatgttagggaaaaataccagacagatcgtattatttttagcccagaatttttaagagaatcaaaagccttatacgataacctttacccaagtcggatcattgtttcttatgaaaaggacgactcaccaagggttattcaggctgctaaagcctttgctggtcttttaaaggaaggagccaaaagcaaggatactccggtcttatttatgggctcacaggaggctgaggcggtcaagctatttgcgaatacctttttggctatgcgggtgtcttactttaatgaattagacacctattccgaaagcaagggtctagatgctcagcgcgtgattgaaggagtctgtcatgatcagcgcattggtaaccattacaataacccttcctttggatatggcggctattgcctgccaaaggacagcaagcagctgttggcaaattatagaggcattccccagtccttgatgtcagcgattgttgaatccaacaagatacgaaaatcttatttggctgaacaaatattagacagagcctctagtcaaaagcaggctggtgtaccattaacgattggcttttaccgcttgattatgaaaagcaactctgataatttccgagaaagcgccattaaagatattattgatatcatcaacgactatggggttaatattgtcatttacgaacccatgcttggcgaggatattggctacagggttgtcaaggacttagagcagttcaaaaacgagtctacaatcattgtgtcaaatcgctttgaggacgacctaggagatgtcattgataaggtttatacgagagatgtctttggaagagactagtcagaaaacgaatggcactcataaggaaccacaaatcaaggaggaactcatgacaaaggtcagaaaagccattatcccagccgccggcctaggcactcgcttcctgcccgccaccaaggcactggccaaggaaatgctcccaatcgtcgataagccaaccattcaattcatcgtcgaggaagccctaaaggcaggtatcgaggagattcttgtcgtcaccggcaaggccaaacgctctatcgaggaccactttgactccaacttcgagctcgaatacaatctccaagccaagggcaaaaccgagctactcaagctcgttgatgagaccactgccatcaacctgcacttcattcgtcagagccaccctagaggactaggggacgctgtcctccaagccaaggcctttgttggcaatgagccctttgtggtcatgctgggggatgacctcatggatattaccaatcctagtgccaagcccttgaccaagcagcttattgaggattatgattgcacacacgcctcaacgattgcagtgatgagggtgccgcatgaggaggtttccaattatggtgtgattgcaccgcaagggaaggctgttaagggcttgtatagtgtggagacctttgttgagaagccaagtccagatgaggcaccgagtgacttagcgattattggtcgatatttgttgacgcctgagatttttgccatattggagaagcaggcgcctggagctggcaatgaggtacagctgaccgatgcgattgacaagctcaataagacacagcgggtttttgcgagggagtttaagggagagcggtatgatgttggggacaagtttggctttatgaagacctcacttgactatgctctcaagcaccctcaggtcaaggacgacctcactgactacattataaagctcagtaagcaactgaacaaggacgtcaagaaataggcgtttattgatcagctattgcagagctatttaaaagcatttagagctttaaggtgggatactagaggattggtatctcactttttaggctgacttgtattaataccaaaagccaaaactaggcagataagcataaggaattagattaaaaataaggaaccaaaacatgaaaaactacgccattatcctagcagctggaaagggaacgcgcatgaagtcagcgcttcccaaggtgctgcacaaggtatcaggcctaagcatgctggagcatgtcctcaagagtgtctcagccctagcccctcaaaagcagctcacagtgatcggtcatcaggcagagcaggtgcgtgctgtcctaggagagcaatcgctaacagtggtgcaagaggagcagctagggacaggccatgcagtcatgatggcagaagaggagctatctggcttagaggggcaaaccctagtgattgcaggtgacacccccttgatcagaggagaaagcctcaaggctctgctagactatcatatcagagaaaagaatgtggcaaccattctcacagccaatgccaaggatccctttggctatggacgaatcattcgcaatgcagcaggagaggtggtcaacatcgttgagcaaaaggatgctaatgaggcagagcaagaggtcaaggagatcaacacagggacttatatctttgacaataagcgcctttttgaggctctaaagcatctcacgactgataatgcccaaggggagtactacctaaccgatgtgatcagtattttcaaggctggccaagaaagggttggcgcttacctgctgaaggactttgatgagagcctaggggttaatgatcgcttagctctagcccaggccgaggtgattatgcaagagcggatcaacaggcagcacatgcttaatggggtgaccctgcaaaacccggcagctacctatattgaaagcagtgtagagattgcaccagacgtcttgattgaagccaatgtgaccttaaagggacagactagaattggcagcagaagtgtcataagcaatgggagctatatccttgattcgaggcttggtgagggtgtagtggttagccagtcggtgattgaggcttcagtcttagcagatggagtgacagtagggccatatgcacacattcgcccggactcccagctcgatgagtgtgttcatattgggaactttgtagaggttaaggggtctcatctaggggccaataccaaggcagggcatttgacttacctggggaatgccgagattggctcagaggttaacattggtgcaggaagcattacggttaattatgatggtcaacggaaataccagacagtgattggcgatcacgcttttattgggagtcattcgactttgatagctccggtagaggttggggagaatgctttaacagcagcagggtctacgatagcccagtcagtgccggcagacagtgtggctatagggcgcagccgtcaggtggtgaaggaaggctatgccaagaggctgccgcaccacccaaatcaagcctaatcgctcaaccaaaagaggcaggtgagaaaacctaggccattaaa

SEQ ID NO: 41 - DNA, 3050 bp, Bacillus thuringiensis
tcgaaacgtaagatgaaaccttagataaaagtgctttttttgttgcaattgaagaattattaatgttaagcttaattaaagataatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatttatgaaaaatcattttatcagtttgaaaattatgtattatgataagaaagggaggaagaaaaatgaatccgaacaatcgaagtgaacatgatacaataaaaactactgaaaataatgaggtgccaactaaccatgttcaatatcctttagcggaaactccaaatccaacactagaagatttaaattataaagagtttttaagaatgactgcagataataatacggaagcactagatagctctacaacaaaagatgtcattcaaaaaggcatttccgtagtaggtgatctcctaggcgtagtaggtttcccgtttggtggagcgcttgtttcgttttatacaaactttttaaatactatttggccaagtgaagacccgtggaaggcttttatggaacaagtagaagcattgatggatcagaaaatagctgattatgcaaaaaataaagctcttgcagagttacagggccttcaaaataatgtcgaagattatgtgagtgcattgagttcatggcaaaaaaatcctgtgagttcacgaaatccacatagccaggggcggataagagagctgttttctcaagcagaaagtcattttcgtaattcaatgccttcgtttgcaatttctggatacgaggttctatttctaacaacatatgcacaagctgccaacacacatttatttttactaaaagacgctcaaatttatggagaagaatggggatacgaaaaagaagatattgctgaattttataaaagacaactaaaacttacgcaagaatatactgaccattgtgtcaaatggtataatgttggattagataaattaagaggttcatcttatgaatcttgggtaaactttaaccgttatcgcagagagatgacattaacagtattagatttaattgcactatttccattgtatgatgttcggctatacccaaaagaagttaaaaccgaattaacaagagacgttttaacagatccaattgtcggagtcaacaaccttaggggctatggaacaaccttctctaatatagaaaattatattcgaaaaccacatctatttgactatctgcatagaattcaatttcacacgcggttccaaccaggatattatggaaatgactctttcaattattggtccggtaattatgtttcaactagaccaagcataggatcaaatgatataatcacatctccattctatggaaataaatccagtgaacctgtacaaaatttagaatttaatggagaaaaagtctatagagccgtagcaaatacaaatcttgcggtctggccgtccgctgtatattcaggtgttacaaaagtggaatttagccaatataatgatcaaacagatgaagcaagtacacaaacgtacgactcaaaaagaaatgttggcgcggtcagctgggattctatcgatcaattgcctccagaaacaacagatgaacctctagaaaagggatatagccatcaactcaattatgtaatgtgctttttaatgcagggtagtagaggaacaatcccagtgttaacttggacacataaaagtgtagacttttttaacatgattgattcgaaaaaaattacacaacttccgttagtaaaggcatataagttacaatctggtgcttccgttgtcgcaggtcctaggtttacaggaggagatatcattcaatgcacagaaaatggaagtgcggcaactatttacgttacaccggatgtgtcgtactctcaaaaatatcgagctagaattcattatgcttctacatctcagataacatttacactcagtttagacggggcaccatttaatcaatactatttcgataaaacgataaataaaggagacacattaacgtataattcatttaatttagcaagtttcagcacaccattcgaattatcagggaataacttacaaataggcgtcacaggattaagtgctggagataaagtttatatagacaaaattgaatttattccagtgaattaaattaactagaaagtaaagaagtagtgaccatctatgatagtaagcaaaggataaaaaaatgagttcataaaatgaataacatagtgttcttcaactttcgctttttgaaggtagatgaagaacactatttttattttcaaaatgaaggaagttttaaatatgtaatcatttaaagggaacaatgaaagtaggaaataagtcattatctataacaaaataacatttttatatagccagaaatgaattataatattaatcttttctaaattgacgtttttctaaacgttctatagcttcaagacgcttagaatcatcaatatttgtatacagagctgttgtttccatcgagttatgtcccatttgattcgctaatagaacaagatctttattttcgttataatgattggttgcataagtatggcgtaatttatgagggcttttcttttcatcaaaagccctcgtgtatttctctgtaagctt

SEQ ID NO: 42 - DNA, 185 bp, Bacillus licheniformis
ggccttaagggcctgcaatcgattgtttgagaaaagaagaagaccataaaaataccttgtctgtcatcagacagggtattttttatgctgtccagactgtccgctgtgtaaaaaaaaggaataaaggggggttgacattattttactgatatgtataatataatttgtataagaaaatggagctc

SEQ ID NO: 43 - DNA, 185 bp, Bacillus licheniformis
ggccttaagggcctgcaatcgattgtttgagaaaagaagaagaccataaaaataccttgtctgtcatcagacagggtattttttatgctgtccagactgtccgctgtgtaaaaaataggaataaaggggggttgacattattttactgatatgtataatataatttgtataagaaaatggagctc

SEQ ID NO: 44 - DNA, 185 bp, Bacillus licheniformis
ggccttaagggcctgcaatcgattgtttgagaaaagaagaagaccataaaaataccttgtctgtcatcagacagggtattttttatgctgtccagactgtccgctgtgtaaaaaataggaataaaggggggttgttattattttactgatatgtaaaatataatttgtataagaaaatggagctc

SEQ ID NO: 45 - DNA, 27 bp, Bacillus
gtccttcttggtacctggaagcagagc

SEQ ID NO: 46 - DNA, 39 bp, Bacillus
gtataaatattcggcccttaaggccagtaccattttccc

SEQ ID NO: 47 - DNA, 59 bp, Bacillus
tggtactggccttaagggccgaatatttatacaatatcatgagctccacattgaaaggg

SEQ ID NO: 48 - DNA, 34 bp, Bacillus
ggtgttctctagagcggccgcggttgcggtcagc

SEQ ID NO: 49 - DNA, 33 bp, Bacillus
ggccttaagggcctgctgtccagactgtccgct

SEQ ID NO: 50 - DNA, 18 bp, Bacillus
ggcgttacaattcaaaga

SEQ ID NO: 51 - DNA, 37 bp, Bacillus licheniformis
ccaggccttaagggccgcatgcgtccttctttgtgct

SEQ ID NO: 52 - DNA, 27 bp, Bacillus licheniformis
gagctcctttcaatgtgatacatatga

SEQ ID NO: 53 - DNA, 24 bp, Bacillus licheniformis
gaattcgacggcttcccgtgcgcc

SEQ ID NO: 54 - DNA, 35 bp, Bacillus licheniformis
gcaagcgagcacggattgtaagtacaagttagata

SEQ ID NO: 55 - DNA, 35 bp, Bacillus licheniformis
aacttgtacttacaatccgtgctcgcttgccgtac

SEQ ID NO: 56 - DNA, 28 bp, Bacillus licheniformis
aagcttccattcaaacctggtgaggaag

SEQ ID NO: 57 - DNA, 30 bp, Bacillus
cgagctcgatgtgttataaattgagaggag

SEQ ID NO: 58 - DNA, 32 bp, Bacillus
gcggccgcgtcataaacgttgcaatcgtgctc

SEQ ID NO: 59 - DNA, 30 bp, Bacillus; identical to SEQ ID NO: 57

SEQ ID NO: 60 - DNA, 19 bp, Bacillus
gtcgaatatgatggggatg

SEQ ID NO: 61 - DNA, 35 bp, Bacillus
ggacaaggacagattgtagcagttgctgatactgg

SEQ ID NO: 62 - DNA, 21 bp, Bacillus
gcgattacagttggggcaacc

SEQ ID NO: 63 - DNA, 23 bp, Bacillus
ggtagcacgacggcatcactaac

SEQ ID NO: 64 - DNA, 27 bp, Bacillus
gtccttcttggtacctggaagcagagc

SEQ ID NO: 65 - DNA, 34 bp, Bacillus
catgctgggcccttaaggccagtaccattttccc

SEQ ID NO: 66 - DNA, 69 bp, Bacillus
cagtaggccttaagggcccagcatgattgagctcaccaccatgggatccgcggccgcacaagggaaggc

SEQ ID NO: 67 - DNA, 24 bp, Bacillus
caattcatcctctagagtctcagg

SEQ ID NO: 68 - DNA, 41 bp, Bacillus
cattctgcagccgcggcaaattccggattttatgtaagcgg

SEQ ID NO: 69 - DNA, 48 bp, Bacillus
catcatatgcggccgcttatcattgaaaaacggtgcttaatctcgaag

SEQ ID NO: 70 - DNA, 18 bp, Bacillus
ggaagtacaaaaataagc

SEQ ID NO: 71 - DNA, 42 bp, Bacillus
catccccccgggagcttaattaaagataatatctttgaattg

SEQ ID NO: 72 - DNA, 24 bp, Bacillus
tgccgcggctgcagaatgaggcag

SEQ ID NO: 73 - DNA, 26 bp, Bacillus
gttaacttgaaaaacggtgcttaatc

(19) United States
(12) Patent Application Publication
(10) Pub. No.: US 2005/0221446 A1
(43) Pub. Date: Oct. 6, 2005

(54) Methods for producing hyaluronic acid in a Bacillus cell

(75) Inventors: William Widner, Davis, CA (US); Alan Sloma, Davis, CA (US); Michael Thomas, Davis, CA (US); Maria Tang, Fairfield, CA (US)

Correspondence Address: NOVOZYMES BIOTECH, INC., 1445 DREW AVE, DAVIS, CA, 95616, US

(73) Assignee: Novozymes Biopolymer A/S., Bagsvaerd (DK)

(21) Appl. No.: 11/096,190

(22) Filed: Mar. 31, 2005

Related U.S. Application Data
(60) Provisional application No. 60/558,507, filed on Mar. 31, 2004.

(51) Int. Cl. C12P 19/28; C12N 1/21

(52) U.S. Cl. 435/85; 435/252.31

(57) ABSTRACT

The present invention relates to methods for producing a hyaluronic acid, comprising: (a) cultivating a Bacillus host cell in a medium conducive for the production of the hyaluronic acid, wherein the Bacillus cell comprises a nucleic acid construct comprising a triple promoter comprising a variant amyL promoter having a mutation corresponding to position 590 of SEQ ID NO: 1, a consensus promoter having the sequence TTGACA for the “−35” region and TATAAT for the “−10” region, and a cryIIIA promoter, in which each promoter sequence of the triple promoter is operably linked to one or more coding sequences involved in the biosynthesis of the hyaluronic acid; and (b) isolating the hyaluronic acid from the cultivation medium. The present invention also relates to Bacillus cells comprising a nucleic acid construct which comprises (i) a triple promoter comprising a variant amyL promoter having a mutation corresponding to position 590 of SEQ ID NO: 1, a consensus promoter having the sequence TTGACA for the “−35” region and TATAAT for the “−10” region, and a cryIIIA promoter, in which each promoter sequence of the triple promoter is operably linked to one or more coding sequences involved in the biosynthesis of the hyaluronic acid.

### CROSS-REFERENCE TO RELATED APPLICATION

This application claims the benefit of U.S. Provisional Application No. 60/558,507, filed Mar. 31, 2004, which application is incorporated herein by reference.

### BACKGROUND OF THE INVENTION

1. Field of the Invention

The present invention relates to methods for producing a hyaluronic acid in a bacterial cell.

2. Description of the Related Art

Hyaluronic acid is an unsulphated glycosaminoglycan composed of repeating disaccharide units of N-acetylglucosamine (GlcNAc) and glucuronic acid (GIcUA) linked together by alternating beta-1,4- and beta-1,3-glycosidic bonds. Numerous roles of hyaluronic acid in the body have been identified (see, Laurent T. C. and Fraser J. R. E., 1992, FASEB J. 6: 2397-2404; and Toole B. P., 1991, “Proteoglycans and hyaluronan in morphogenesis and differentiation.” In: Cell Biology of the Extracellular Matrix, pp. 305-341, Hay E. D., ed., Plenum, New York). Hyaluronic acid is present in hyaline cartilage, synovial joint fluid, and skin tissue, both dermis and epidermis. Hyaluronic acid is suspected of having a role in numerous physiological functions, such as adhesion, development, cell motility, cancer, angiogenesis, and wound healing. Due to the unique physical and biological properties of hyaluronic acid, it is employed in eye and joint surgery. Products of hyaluronic acid have also been developed for use in orthopedics, rheumatology, and dermatology.

Rooster combs have been the conventional source for hyaluronic acid. However, the use of recombinant microorganisms containing genes for the biosynthesis of hyaluronic acid is emerging as an alternative.

Bacilli are well established as host cell systems for the production of native and recombinant proteins. U.S. Patent Application No. 2002/0160489 discloses the construction of three Bacillus subtilis strains to contain one or both of the Streptococcus pyogenes genes for hyaluronan synthase and UDP-glucose dehydrogenase. U.S. Patent Application No. 2003/0092118 describes the use of recombinant Bacillus host cells comprising a hyaluronan synthase gene from Streptococcus equisimilis, Streptococcus pyogenes, Streptococcus uberis, Pasteurella multocida, Sulfolobus solfactaricus, Bacillus anthracis pXO1, Paramecium bursaria Chlorella virus, or Ectocarpus siliculosus virus under control of a promoter for the production of hyaluronic acid. WO 03/054163 discloses methods for producing hyaluronic acid by cultivating a Bacillus host cell comprising a nucleic acid construct comprising a hyaluronan synthase encoding sequence operably linked to a promoter sequence foreign to the hyaluronan synthase encoding sequence.

U.S. Pat. Nos. 6,255,076 and 5,955,310 describe tandem promoters and constructs and methods for use in the expression of enzymes in Bacillus cells. The use of the cryIIIA stabilizer sequence for improved production in Bacillus is also described therein. WO 03/095658 discloses a triple promoter composed of amyL4199, short consensus amyQ, and cryIIIA promoter sequences.

It is an object of the present invention to provide improved methods for producing hyaluronic acid in a Bacillus strain.

### SUMMARY OF THE INVENTION

The present invention relates to methods for producing a hyaluronic acid, comprising: (a) cultivating a Bacillus host cell in a medium conducive for the production of the hyaluronic acid, wherein the Bacillus cell comprises a nucleic acid construct comprising a triple promoter comprising a variant amyL promoter having a mutation corresponding to position 590 of SEQ ID NO: 1, a consensus promoter having the sequence TTGACA for the “−35” region and TATAAT for the “−10” region, and a cryIIIA promoter, in which each promoter sequence of the triple promoter is operably linked to one or more coding sequences involved in the biosynthesis of the hyaluronic acid; and (b) isolating the hyaluronic acid from the cultivation medium. In a preferred aspect, the nucleic acid construct further comprises an mRNA processing/stabilizing sequence located downstream of the triple promoter and upstream of the one or more coding sequences involved in the biosynthesis of the hyaluronic acid.

The present invention also relates to Bacillus cells comprising a nucleic acid construct which comprises a triple promoter comprising a variant amyL promoter having a mutation corresponding to position 590 of SEQ ID NO: 1, a consensus promoter having the sequence TTGACA for the “−35” region and TATAAT for the “−10” region, and a cryIIIA promoter, in which each promoter sequence of the triple promoter is operably linked to one or more coding sequences involved in the biosynthesis of a hyaluronic acid. In a preferred aspect, the nucleic acid construct further comprises an mRNA processing/stabilizing sequence located downstream of the triple promoter and upstream of the one or more coding sequences involved in the biosynthesis of the hyaluronic acid.

The present invention also relates to methods for producing a selectable marker-free mutant of a Bacillus cell, comprising deleting a selectable marker gene of the Bacillus cell, wherein the Bacillus cell comprises a nucleic acid construct comprising a triple promoter comprising a variant amyL promoter having a mutation corresponding to position 590 of SEQ ID NO: 1, a consensus promoter having the sequence TTGACA for the “−35” region and TATAAT for the “−10” region, and a cryIIIA promoter, in which each promoter sequence of the triple promoter is operably linked to one or more coding sequences involved in the biosynthesis of a hyaluronic acid.

The present invention also relates to methods for obtaining a Bacillus host cell, comprising introducing into a Bacillus cell a nucleic acid construct comprising a triple promoter comprising a variant amyL promoter having a mutation corresponding to position 590 of SEQ ID NO: 1, a consensus promoter having the sequence TTGACA for the “−35” region and TATAAT for the “−10” region, and a cryIIIA promoter, in which each promoter sequence of the triple promoter is operably linked to one or more coding sequences involved in the biosynthesis of a hyaluronic acid.

The present invention further relates to nucleic acid constructs comprising a triple promoter comprising a variant amyL promoter having a mutation corresponding to position 590 of SEQ ID NO: 1, a consensus promoter having the sequence TTGACA for the “−35” region and TATAAT for the “−10” region, and a cryIIIA promoter, in which each promoter sequence of the triple promoter is operably linked to one or more coding sequences involved in the biosynthesis of a hyaluronic acid.

### BRIEF DESCRIPTION OF THE FIGURES

FIG. 1 shows a restriction map of pNBT28.

FIG. 2 shows a restriction map of pMRT038.

FIG. 3 shows a restriction map of pNBT29.

FIG. 4 shows a restriction map of pWWi001.1.

FIG. 5 shows a restriction map of pWWi005.

FIG. 6 shows a restriction map of pNBT30.

FIG. 7 shows a restriction map of pNBT31.

FIG. 8 shows a restriction map of pNBT33.

FIG. 9 shows a restriction map of pMDT006.

FIG. 10 shows a restriction map of pMDT007.

FIG. 11 shows a restriction map of pNBT37.

FIG. 12 shows a restriction map of pNBT38.

FIG. 13 shows a restriction map of pNBT39.

FIG. 14 shows a restriction map of pMRT040.

FIG. 15 shows a restriction map of pMRT044.

FIG. 16 shows a restriction map of pMRT070.

FIG. 17 shows a restriction map of pMRT075.

FIG. 18 shows a restriction map of pNBT40.

FIG. 19 shows a restriction map of pMRT077.

FIG. 20 shows a restriction map of pTH012.

FIG. 21 shows a restriction map of pMB1024-1.

FIG. 22 shows a restriction map of pMB1242.

FIG. 23 shows a restriction map of pTH029.

FIG. 24 shows a restriction map of pTH026.

FIG. 25 shows a restriction map of pTH013.

FIG. 26 shows a restriction map of pTH020.

FIG. 27 shows the production of hyaluronic acid by Bacillus licheniformis TH15.

### DETAILED DESCRIPTION OF THE INVENTION

The present invention relates to methods for producing a hyaluronic acid, comprising: (a) cultivating a Bacillus cell in a medium conducive for the production of the hyaluronic acid, wherein the Bacillus cell comprises a nucleic acid construct comprising a triple promoter comprising a variant amyL promoter having a mutation corresponding to position 590 of SEQ ID NO: 1, a consensus promoter having the sequence TTGACA for the “−35” region and TATAAT for the “−10” region, and a cryIIIA promoter, in which each promoter sequence of the triple promoter is operably linked to one or more coding sequences involved in the biosynthesis of the hyaluronic acid; and (b) isolating the hyaluronic acid from the cultivation medium. In the methods of the present invention, it is preferred that the nucleic acid construct further comprises an mRNA processing/stabilizing sequence located downstream of the triple promoter and upstream of the one or more coding sequences involved in the biosynthesis of the hyaluronic acid.

Hyaluronic Acid

“Hyaluronic acid” is defined herein as an unsulphated glycosaminoglycan composed of repeating disaccharide units of N-acetylglucosamine (GIcNAc) and glucuronic acid (GIcUA) linked together by alternating beta-1,4- and beta-1,3-glycosidic bonds. Hyaluronic acid is also known as hyaluronan, hyaluronate, or HA, and are used interchangeably herein.

In a preferred aspect, the hyaluronic acid obtained by the methods of the present invention has a molecular weight of about 10,000 to about 10,000,000 Da. In a more preferred aspect, the hyaluronic acid obtained by the methods of the present invention has a molecular weight of about 25,000 to about 5,000,000 Da. In a most preferred aspect, the hyaluronic acid obtained by the methods of the present invention has a molecular weight of about 50,000 to about 3,000,000 Da.

The level of hyaluronic acid produced by a Bacillus host cell of the present invention may be determined according to the modified carbazole method (Bitter and Muir, 1962, Anal Biochem. 4: 330-334). Moreover, the average molecular weight of the hyaluronic acid may be determined using standard methods in the art, such as those described by Ueno et al., 1988, Chem. Pharm. Bull. 36: 4971-4975; Wyatt, 1993, Anal. Chim. Acta 272: 1-40; and Wyatt Technologies, 1999, “Light Scattering University DAWN Course Manual” and “DAWN EOS Manual”, Wyatt Technology Corporation, Santa Barbara, Calif.

The hyaluronic acid obtained by the methods of the present invention may be subjected to various techniques known in the art to modify the hyaluronic acid, such as crosslinking as described, for example, in U.S. Pat. Nos. 5,616,568, 5,652,347, and 5,874,417. Moreover, the molecular weight of the hyaluronic acid may be altered using techniques known in the art.

Host Cells

The present invention also relates to Bacillus cells comprising a triple promoter comprising a variant amyL promoter having a mutation corresponding to position 590 of SEQ ID NO: 1, a consensus promoter having the sequence TTGACA for the “−35” region and TATAAT for the “−10” region, and a cryIIIA promoter, in which each promoter sequence of the triple promoter is operably linked to one or more coding sequences involved in the biosynthesis of a hyaluronic acid. In a preferred aspect, the nucleic acid construct further comprises an mRNA processing/stabilizing sequence located downstream of the triple promoter and upstream of the one or more coding sequences involved in the biosynthesis of a hyaluronic acid. In another preferred aspect, the Bacillus cell is free of a foreign or heterologous selectable marker gene.

The present invention also relates to methods for obtaining a Bacillus host cell, comprising introducing into a Bacillus cell a nucleic acid construct comprising a triple promoter comprising a variant amyL promoter having a mutation corresponding to position 590 of SEQ ID NO: 1, a consensus promoter having the sequence TTGACA for the “−35” region and TATAAT for the “−10” region, and a cryIIIA promoter, in which each promoter sequence of the triple promoter is operably linked to one or more coding sequences involved in the biosynthesis of a hyaluronic acid. In a preferred aspect, the nucleic acid construct further comprises an mRNA processing/stabilizing sequence located downstream of the triple promoter and upstream of the one or more coding sequences involved in the biosynthesis of a hyaluronic acid.

In the methods of the present invention, the Bacillus host cell may be any Bacillus cell suitable for recombinant production of a hyaluronic acid. The Bacillus host cell may be a wild-type Bacillus cell or a mutant thereof. Bacillus cells useful in the practice of the present invention include, but are not limited to, Bacillus agaraderhens, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. Mutant Bacillus subtilis cells particularly adapted for recombinant expression are described in WO 98/22598. Non-encapsulating Bacillus cells are particularly useful in the present invention.

In a preferred aspect, the Bacillus host cell is a Bacillus amyloliquefaciens, Bacillus clausii, Bacillus lentus, Bacillus licheniformis, Bacillus stearothermophilus or Bacillus subtilis cell. In a more preferred aspect, the Bacillus cell is a Bacillus amyloliquefaciens cell. In another more preferred aspect, the Bacillus cell is a Bacillus clausii cell. In another more preferred aspect, the Bacillus cell is a Bacillus lentus cell. In another more preferred aspect, the Bacillus cell is a Bacillus licheniformis cell. In another more preferred aspect, the Bacillus cell is a Bacillus subtilis cell. In a most preferred aspect, the Bacillus host cell is Bacillus subtilis A164Δ5 or Bacillus subtilis 168Δ4 (see U.S. Pat. No. 5,891,701). In another most preferred aspect, the Bacillus host cell is Bacillus licheniformis SJ1904 (see U.S. Pat. No. 5,733,753).

Transformation of a Bacillus host cell with a nucleic acid construct of the present invention may, for instance, be effected by protoplast transformation (see, for example, Chang and Cohen, 1979, Molecular General Genetics 168: 111-115), by using competent cells (see, for example, Young and Spizizen, 1961, Journal of Bacteriology 81: 823-829, or Dubnau and Davidoff-Abelson, 1971, Journal of Molecular Biology 56: 209-221), by electroporation (see, for example, Shigekawa and Dower, 1988, Biotechniques 6: 742-751), or by conjugation (see, for example, Koehler and Thorne, 1987, Journal of Bacteriology 169: 5271-5278).

Nucleic Acid Constructs

The construction of a Bacillus cell comprising a triple promoter or a triple promoter and an mRNA processing/stabilizing sequence, operably linked to a one or more genes involved in the biosynthesis of a hyaluronic acid may be accomplished by modifying the one or more genes using methods well known in the art to operably link the triple promoter and, alternatively also, the mRNA processing/stabilizing sequence to the one or more genes, inserting the construct into a vector, and introducing the vector into the Bacillus cell's chromosome by homologous recombination or into the Bacillus cell as an extrachromosomal autonomously replicating element, e.g., plasmid. However, it will be understood that the one or more genes may also be manipulated in vivo in the Bacillus cell using methods well known in the art.

“Nucleic acid construct” is defined herein as a nucleic acid molecule, either single- or double-stranded, which is isolated from a naturally occurring gene or which has been modified to contain segments of nucleic acid which are combined and juxtaposed in a manner which would not otherwise exist in nature. The term nucleic acid construct may be synonymous with the term expression cassette when the nucleic acid construct contains all the control sequences required for expression of a coding sequence.

“Promoter” is defined herein as a nucleotide sequence involved in the binding of RNA polymerase to initiate transcription of a gene.

“Triple promoter” is defined herein as three promoter sequences in tandem each of which is operably linked to a coding sequence or coding sequences and mediates the transcription of the coding sequence or coding sequences into mRNA.

“Operably linked” is defined herein as a configuration in which a control sequence, e.g., a triple promoter, is appropriately placed at a position relative to a coding sequence such that the control sequence directs the production of a hyaluronic acid encoded by the coding sequence.

“Coding sequence” is defined herein as a nucleotide sequence which is transcribed into mRNA and translated into an enzyme (or other protein) involved in the biosynthesis of a hyaluronic acid when placed under the control of the appropriate control sequences. The boundaries of the coding sequence are generally determined by a ribosome binding site located just upstream of the open reading frame at the 5′ end of the mRNA and a transcription terminator sequence located just downstream of the open reading frame at the 3′ end of the mRNA. A coding sequence can include, but is not limited to, genomic DNA, cDNA, semisynthetic, synthetic, and recombinant nucleic acids.

The techniques used to isolate or clone a gene encoding a polypeptide, e.g., enzyme, are well known in the art and include, for example, isolation from genomic DNA, preparation from cDNA, or a combination thereof. The cloning of the gene from such genomic DNA can be effected, e.g., by using antibody screening of expression libraries to detect cloned DNA fragments with shared structural features or the well known polymerase chain reaction (PCR). See, for example, Innis et al., 1990, PCR Protocols: A Guide to Methods and Application, Academic Press, New York. Other nucleic acid amplification procedures such as ligase chain reaction, ligated activated transcription, and nucleic acid sequence-based amplification may be used. The cloning procedures may involve excision and isolation of a desired nucleic acid fragment comprising the gene encoding the polypeptide, insertion of the fragment into a vector molecule, and incorporation of the recombinant vector into a Bacillus cell where clones of the nucleotide sequence will be replicated. The gene may be of genomic, cDNA, RNA, semi-synthetic, synthetic origin, or any combinations thereof.

An isolated gene encoding an enzyme (or other protein) involved in the biosynthesis of a hyaluronic acid may be manipulated in a variety of ways to provide for expression of the enzyme (or other protein). Manipulation of the gene's sequence prior to its insertion into a construct or vector may be desirable or necessary depending on the expression vector or Bacillus host cell. The techniques for modifying nucleotide sequences utilizing cloning methods are well known in the art. It will be understood that the sequence of the gene may also be manipulated in vivo in the host cell using methods well known in the art.

A number of enzymes are involved in the biosynthesis of hyaluronic acid. In the methods of the present invention, the one or more genes involved in the biosynthesis of a hyaluronic acid include, but are not limited to, genes encoding hyaluronan synthase, UDP-glucose 6-dehydrogenase, UDP-glucose pyrophosphorylase, UDP-N-acetylglucosamine pyrophosphorylase, glucose-6-phosphate isomerase, hexokinase, phosphoglucomutase, amidotransferase, mutase, and acetyl transferase. Hyaluronan synthase is the key enzyme in the production of hyaluronic acid.

“Hyaluronan synthase” is defined herein as a synthase that catalyzes the elongation of a hyaluronan chain by the addition of GIcUA and GIcNAc sugar precursors. The amino acid sequences of streptococcal hyaluronan synthases, vertebrate hyaluronan synthases, and viral hyaluronan synthases are distinct from the Pasteurella hyaluronan synthase, and have been proposed for classification as Group I and Group II hyaluronan synthases, the Group I hyaluronan synthases including Streptococcal hyaluronan synthases (DeAngelis, 1999, Cell. Mol. Life Sci. 56: 670-682). For production of a hyaluronan in Bacillus host cells, hyaluronan synthases of an eukaryotic origin, such as mammalian hyaluronan synthases, may be used, but are less preferred.

The hyaluronan synthase gene may be any hyaluronan synthase gene capable of being expressed in a Bacillus host cell. The gene may be of any origin. Preferred hyaluronan synthase genes include any of either Group I or Group II, such as the Group I hyaluronan synthase genes from Streptococcus equisimilis, Streptococcus pyogenes, Streptococcus uberis, and Streptococcus equi subsp. Zooepidemicus, or the Group II hyaluronan synthase gene of Pasturella multocida.

The nucleotide sequences disclosed herein or a subsequence thereof, as well as the amino acid sequence thereof or a fragment thereof, may be used to design a nucleic acid probe to identify and clone DNA encoding enzymes involved in the biosynthesis of a hyaluronic acid from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, preferably at least 25, more preferably at least 35, and most preferably at least 70 nucleotides in length. Longer probes can also be used. Both DNA and RNA probes can be used. The probes are typically labeled for detecting the corresponding gene (for example, with ³²P, ³H, ³⁵S, biotin, or avidin).

Thus, a genomic DNA or cDNA library prepared from such other organisms may be screened for DNA which hybridizes with the probes described above and which encodes an enzyme in the biosynthetic pathway of hyaluronic acid. Genomic or other DNA from such other organisms may be separated by agarose or polyacrylamide gel electrophoresis, or other separation techniques. DNA from the libraries or the separated DNA may be transferred to and immobilized on nitrocellulose or other suitable carrier material. In order to identify a clone or DNA which is homologous with the nucleotide sequences disclosed herein or subsequences thereof, the carrier material is used in a Southern blot. For purposes of the present invention, hybridization indicates that the nucleic acid sequence hybridizes to a labeled nucleic acid probe corresponding to the nucleotide sequences disclosed herein, its complementary strand, or a subsequence thereof, under very low to very high stringency conditions. Molecules to which the nucleic acid probe hybridizes under these conditions can be detected using X-ray film.

For long probes of at least 100 nucleotides in length, very low to very high stringency conditions are defined as prehybridization and hybridization at 42° C. in 5×SSPE, 0.3% SDS, 200 μg/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

For short probes which are about 15 nucleotides to about 70 nucleotides in length, stringency conditions are defined as prehybridization, hybridization, and washing post-hybridization at about 5° C. to about 10° C. below the calculated Tₘ using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1× Denhardt's solution, 1 mM sodium pyrophosphate, 1 mM sodium monobasic phosphate, 0.1 mM ATP, and 0.2 mg of yeast RNA per ml following standard Southern blotting procedures for 12 to 24 hours optimally.

For short probes which are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

In a preferred aspect, the hyaluronan synthase gene is a Group I hyaluronan synthase gene.

In a more preferred aspect, the Group I hyaluronan synthase gene is selected from the group consisting of (a) a gene encoding a hyaluronan synthase with an amino acid sequence having at least 70%, preferably at least 75%, more preferably at least 80%, more preferably at least 85%, even more preferably at least 90%, most preferably at least 95%, or even most preferably at least 97% identity to SEQ ID NO: 3, SEQ ID NO: 5, or SEQ ID NO: 7; (b) a gene which hybridizes under low, medium, medium-high, or high stringency conditions with SEQ ID NO: 2, SEQ ID NO: 4, or SEQ ID NO: 6; and (c) a complementary strand of (a) or (b). For purposes of the present invention, the degree of identity between two amino acid sequences is determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters were Ktuple=1, gap penalty=3, windows=5, and diagonals=5.

In a most preferred aspect, the Group I hyaluronan synthase gene encodes a hyaluronan synthase having the amino acid sequence of SEQ ID NO: 3, SEQ ID NO: 5, or SEQ ID NO: 7; or a fragment thereof having hyaluronan synthase activity.

In another preferred aspect, the hyaluronan synthase gene is a Group II hyaluronan synthase gene.

In a more preferred aspect, the Group II hyaluronan synthase gene is selected from the group consisting of (a) a gene encoding a hyaluronan synthase with an amino acid sequence having at least 70%, preferably at least 75%, more preferably at least 80%, more preferably at least 85%, even more preferably at least 90%, most preferably at least 95%, or even most preferably at least 97% identity to SEQ ID NO: 9; (b) a gene which hybridizes under low, medium, medium-high, or high stringency conditions with SEQ ID NO: 8; and (c) a complementary strand of (a) or (b).

In a most preferred aspect, the Group II hyaluronan synthase gene encodes a hyaluronan synthase having the amino acid sequence of SEQ ID NO: 9, or a fragment thereof having hyaluronan synthase activity.

Other hyaluronan synthase genes that may be used in the present invention are hyaluronan synthase genes from Bacillus anthracis, Sulfolobus solfataricus, Ectocarpus siliculosus virus, and Paramecium bursaria Chlorella virus (PBCV-1).

In another preferred aspect, the hyaluronan synthase gene is selected from the group consisting of (a) a gene encoding a hyaluronan synthase with an amino acid sequence having at least 70%, preferably at least 75%, more preferably at least 80%, more preferably at least 85%, even more preferably at least 90%, most preferably at least 95%, or even most preferably at least 97% identity to SEQ ID NO: 11, SEQ ID NO: 13, SEQ ID NO: 15, or SEQ ID NO: 17; (b) a gene which hybridizes under low, medium, medium-high, or high stringency conditions with SEQ ID NO: 10, SEQ ID NO: 12, SEQ ID NO: 14, or SEQ ID NO: 16; and (c) a complementary strand of (a) or (b).

In a more preferred aspect, the hyaluronan synthase gene encodes a hyaluronan synthase having the amino acid sequence of SEQ ID NO: 11, SEQ ID NO: 13, SEQ ID NO: 15, or SEQ ID NO: 17; or a fragment thereof having hyaluronan synthase activity.

The methods of the present invention also include nucleic constructs whereby precursor sugars of hyaluronan are supplied to the host cell by being encoded by endogenous genes, by non-endogenous genes, or by a combination of endogenous and non-endogenous genes present in a construct. The precursor sugar may be D-glucuronic acid or N-acetyl-glucosamine.

In the methods of the present invention, the nucleic acid construct may further comprise one or more genes encoding enzymes involved in the biosynthesis of a precursor sugar of a hyaluronan. Alternatively, the Bacillus host cell may further comprise one or more second nucleic acid constructs comprising one or more genes encoding enzymes involved in the biosynthesis of a precursor sugar. Hyaluronan production is improved by the use of constructs with a gene or genes directing a step in the biosynthetic pathway of a precursor sugar of hyaluronan. The phrase “directing a step in the biosynthetic pathway of a precursor sugar of hyaluronan” means herein that the expressed enzyme of the gene is active in the formation of N-acetyl-glucosamine or D-glucuronic acid, or a sugar that is a precursor of either of N-acetyl-glucosamine and D-glucuronic acid.

In a preferred method for supplying precursor sugars, constructs are provided for improving hyaluronan production in a host cell naturally containing a hyaluronan synthase gene, by culturing a host cell having a recombinant construct with a triple promoter operably linked to one or more genes encoding enzymes in the biosynthetic pathway of a precursor sugar of hyaluronan. In a preferred method, the host cell also comprises a recombinant construct having a triple promoter operably linked to a hyaluronan synthase. Thus, the present invention also relates to constructs for improving hyaluronan production by the use of constructs with one or more genes directing a step in the biosynthetic pathway of a precursor sugar of hyaluronan. Such genes in the nucleic acid constructs are operably linked to a triple promoter as described herein.

The genes involved in the biosynthesis of precursor sugars for the production of hyaluronic acid include a UDP-glucose 6-dehydrogenase gene, UDP-glucose pyrophosphorylase gene, UDP-N-acetylglucosamine pyrophosphorylase gene, glucose-6-phosphate isomerase gene, hexokinase gene, phosphoglucomutase gene, amidotransferase gene, mutase gene, and acetyl transferase gene.

In a cell containing a hyaluronan synthase gene, any one or combination of two or more of hasB, hasC, and hasD, or homologs thereof, such as the Bacillus subtilis tuaD, gtaB, and gcaD, respectively, as well as hasE, may be expressed to increase the pools of precursor sugars available to the hyaluronan synthase. The Bacillus subtilis genome is described in Kunst, et al., Nature 390, 249-256, “The complete genome sequence of the Gram-positive bacterium Bacillus subtilis” (20 Nov. 1997). In some instances, such as where the host cell does not have, for example, a native hyaluronan synthase activity, the construct further includes a hasA gene.

The genes encoding the biosynthetic enzymes may be native to the host cell, while in other cases heterologous genes may be utilized, or a combination of native and heterologous genes may be used. If one or more genes are included in a construct, they may be genes that are associated with one another in a native operon, such as the genes of the HAS operon of Streptococcus equisimilis, which comprises hasA, hasB, hasC and hasD. In other instances, the use of some combination of the precursor genes may be desired, without all components of the operon included. The use of some genes native to the host cell, and others which are exogenous, may also be preferred in other cases. The choice will depend on the available pools of sugars in a given host cell, the ability of the cell to accommodate overproduction without interfering with other functions of the host cell, and whether the cell regulates expression from its native genes differently than exogenous genes.

As one example, depending on the metabolic requirements and growth conditions of the cell, and the available precursor sugar pools, it may be desirable to increase production of N-acetyl-glucosamine by expression of a gene encoding UDP-N-acetylglucosamine pyrophosphorylase, such as the hasD gene, the Bacillus gcaD gene, or homologs thereof. Alternatively, the precursor sugar may be D-glucuronic acid. In one such aspect, the gene encodes UDP-glucose 6-dehydrogenase. Such genes include the Bacillus tuaD gene, the hasB gene of Streptococcus, or homologs thereof. Another gene may encode UDP-glucose pyrophosphorylase, such as the Bacillus gtaB gene, the hasC gene of Streptococcus, or homologs thereof.

In the methods of the present invention, the UDP-glucose 6-dehydrogenase gene may be a hasB gene or tuaD gene; or homologs thereof.

In a preferred aspect, the hasB gene is selected from the group consisting of (a) a gene encoding a UDP-glucose 6-dehydrogenase with an amino acid sequence having at least 70%, preferably at least 75%, more preferably at least 80%, more preferably at least 85%, even more preferably at least 90%, most preferably at least 95%, or even most preferably at least 97% identity to SEQ ID NO: 19, SEQ ID NO: 21, or SEQ ID NO: 23; (b) a gene which hybridizes under low, medium, medium-high, or high stringency conditions with SEQ ID NO: 18, SEQ ID NO: 20, or SEQ ID NO: 22; and (c) a complementary strand of (a) or (b).

In a more preferred aspect, the hasB gene encodes a UDP-glucose 6-dehydrogenase having the amino acid sequence of SEQ ID NO: 19, SEQ ID NO: 21, or SEQ ID NO: 23; or a fragment thereof having UDP-glucose 6-dehydrogenase activity.

In another preferred aspect, the tuaD gene is selected from the group consisting of (a) a nucleotide sequence encoding a polypeptide with an amino acid sequence having at least 70%, preferably at least 75%, more preferably at least 80%, more preferably at least 85%, even more preferably at least 90%, most preferably at least 95%, or even most preferably at least 97% identity to SEQ ID NO: 25; (b) a nucleotide sequence which hybridizes under low, medium, medium-high, or high stringency conditions with SEQ ID NO: 24; and (c) a complementary strand of (a) or (b).

In another more preferred aspect, the tuaD gene encodes a UDP-glucose 6-dehydrogenase having the amino acid sequence of SEQ ID NO: 25, or a fragment thereof having UDP-glucose 6-dehydrogenase activity.

In the methods of the present invention, the UDP-glucose pyrophosphorylase gene may be a hasC gene or gtaB gene; or homologs thereof.

In a preferred aspect, the hasC gene is selected from the group consisting of (a) a gene encoding a UDP-glucose pyrophosphorylase with an amino acid sequence having at least 70%, preferably at least 75%, more preferably at least 80%, more preferably at least 85%, even more preferably at least 90%, most preferably at least 95%, or even most preferably at least 97% identity to SEQ ID NO: 27, SEQ ID NO: 29, or SEQ ID NO: 31; (b) a gene which hybridizes under low, medium, medium-high, or high stringency conditions with SEQ ID NO: 26 or SEQ ID NO: 28, or SEQ ID NO: 30; and (c) a complementary strand of (a) or (b).

In another more preferred aspect, the hasC gene encodes a UDP-glucose pyrophosphorylase having the amino acid sequence of SEQ ID NO: 27 or SEQ ID NO: 29, or SEQ ID NO: 31; or a fragment thereof having UDP-glucose pyrophosphorylase activity.

In another preferred aspect, the gtaB gene is selected from the group consisting of (a) a gene encoding a UDP-glucose pyrophosphorylase with an amino acid sequence having at least 70%, preferably at least 75%, more preferably at least 80%, more preferably at least 85%, even more preferably at least 90%, most preferably at least 95%, or even most preferably at least 97% identity to SEQ ID NO: 33; (b) a gene which hybridizes under low, medium, medium-high, or high stringency conditions with SEQ ID NO: 32; and (c) a complementary strand of (a) or (b).

In another more preferred aspect, the gtaB gene encodes a UDP-glucose pyrophosphorylase having the amino acid sequence of SEQ ID NO: 33, or a fragment thereof having UDP-glucose pyrophosphorylase activity.

In the methods of the present invention, the UDP-N-acetylglucosamine pyrophosphorylase gene may be a hasD or gcaD gene; or homologs thereof.

In a preferred aspect, the hasD gene is selected from the group consisting of (a) a gene encoding a UDP-N-acetylglucosamine pyrophosphorylase with an amino acid sequence having at least 70%, preferably at least 75%, more preferably at least 80%, more preferably at least 85%, even more preferably at least 90%, most preferably at least 95%, or even most preferably at least 97% identity to SEQ ID NO: 35; (b) a gene which hybridizes under low, medium, medium-high, or high stringency conditions with SEQ ID NO: 34; and (c) a complementary strand of (a) or (b).

In another more preferred aspect, the hasD gene encodes a UDP-N-acetylglucosamine pyrophosphorylase having the amino acid sequence of SEQ ID NO: 35, or a fragment thereof having UDP-N-acetylglucosamine pyrophosphorylase activity.

In another preferred aspect, the gcaD gene is selected from the group consisting of (a) a gene encoding a UDP-N-acetylglucosamine pyrophosphorylase with an amino acid sequence having at least 70%, preferably at least 75%, more preferably at least 80%, more preferably at least 85%, even more preferably at least 90%, most preferably at least 95%, or even most preferably at least 97% identity to SEQ ID NO: 37; (b) a gene which hybridizes under low, medium, medium-high, or high stringency conditions with SEQ ID NO: 36; and (c) a complementary strand of (a) or (b).

In another more preferred aspect, the gcaD gene encodes a UDP-N-acetylglucosamine pyrophosphorylase having the amino acid sequence of SEQ ID NO: 37, or a fragment thereof having UDP-N-acetylglucosamine pyrophosphorylase activity.

In the methods of the present invention, the glucose-6-phosphate isomerase gene may be a hasE or homolog thereof.

In a preferred aspect, the hasE gene is selected from the group consisting of (a) a gene encoding a glucose-6-phosphate with an amino acid sequence having at least 70%, preferably at least 75%, more preferably at least 80%, more preferably at least 85%, even more preferably at least 90%, most preferably at least 95%, or even most preferably at least 97% identity to SEQ ID NO: 39; (b) a gene which hybridizes under low, medium, medium-high, or high stringency conditions with SEQ ID NO: 38; and (c) a complementary strand of (a) or (b).

In another more preferred aspect, the hasE gene encodes a glucose-6-phosphate having the amino acid sequence of SEQ ID NO: 39, or a fragment thereof having glucose-6-phosphate isomerase activity.

The present invention also relates to a nucleic acid construct comprising an isolated polynucleotide encoding a hyaluronan synthase operon comprising a hyaluronan synthase gene and a UDP-glucose 6-dehydrogenase gene, and optionally one or more genes selected from the group consisting of a UDP-glucose pyrophosphorylase gene, UDP-N-acetylglucosamine pyrophosphorylase gene, and glucose-6-phosphate isomerase gene.

“Artificial operons” can be constructed to mimic the operons of Streptococcus equisimilis (WO 03/054163) or Streptococcus pyogenes (Crater and van de Rijn, 1995, J. Biol. Chem. 270: 18452-18458). Such artificial operons comprise hasA, hasB, hasC, and hasD, or homologs thereof, or, alternatively, may include less than the full complement present in the Streptococcus equisimilis operon. The artificial operons may also comprise a glucose-6-phosphate isomerase gene (hasE) as well as one or more genes selected from the group consisting of a hexokinase gene, phosphoglucomutase gene, amidotransferase gene, mutase gene, and acetyl transferase gene. A polynucleotide encoding most of the hyaluronan synthase operon of Streptococcus equisimilis is found in SEQ ID NO: 40. This sequence contains the hasB (SEQ ID NO: 18) and hasC (SEQ ID NO: 26) homologs of the Bacillus subtilis tuaD gene (SEQ ID NO: 24) and gtaB gene (SEQ ID NO: 32), respectively, as is the case for Streptococcus pyogenes, as well as a homolog of the gcaD gene (SEQ ID NO: 36), which has been designated hasD (SEQ ID NO: 34). The Bacillus subtilis gcaD gene encodes UDP-N-acetylglucosamine pyrophosphorylase, which is involved in the synthesis of N-acetyl-glucosamine, one of the two components of hyaluronan. The Streptococcus equisimilis homolog of gcaD, hasD, is arranged by Streptococcus equisimilis on the hyaluronan synthase operon. The polynucleotide also contains a portion of the hasA gene (the last 1156 bp of SEQ ID NO: 2).

In a preferred aspect, the nucleic acid construct comprises one or more genes selected from the group consisting of hasA, hasB, tuaD, hasC, gtaB, hasD, gcaD, and hasE.

In another preferred aspect, the nucleic acid construct comprises hasA.

In another preferred aspect, the nucleic acid construct comprises hasA and hasB or tuaD. In another preferred aspect, the nucleic acid construct comprises hasA and hasC or gtaB. In another preferred aspect, the nucleic acid construct comprises hasA and hasD or gcaD. In another preferred aspect, the nucleic acid construct comprises hasA and hasE. In another preferred aspect, each of the nucleic acid constructs described above do not comprise hasA.

In another preferred aspect, the nucleic acid construct comprises hasA, hasB or tuaD, and hasC or gtaB. In another preferred aspect, the nucleic acid construct comprises hasA, hasB or tuaD, and hasD or gcaD. In another preferred aspect, the nucleic acid construct comprises hasA, hasB or tuaD, and hasE. In another preferred aspect, the nucleic acid construct comprises hasA, hasC or gtaB, and hasD or gcaD. In another preferred aspect, the nucleic acid construct comprises hasA, hasC or gtaB, and hasE. In another preferred aspect, each of the nucleic acid constructs described above do not comprise hasA.

In another preferred aspect, the nucleic acid construct comprises hasA, hasB or tuaD, hasC or gtaD, and hasD. In another preferred aspect, the nucleic acid construct comprises hasA, hasB, hasD or gcaD, and hasE. In another preferred aspect, the nucleic acid construct comprises hasA, hasC or gtaD, hasD or gcaD, and hasE. In another preferred aspect, the nucleic acid construct comprises hasA, hasB or tuaD, hasC or gtaD, and hasE. In another preferred aspect, each of the nucleic acid constructs described above do not comprise hasA.

Based on the above preferred aspects, the genes noted can be replaced with other homologs thereof.

In the methods of the present invention, the nucleic acid constructs comprise one or more genes involved in the biosynthesis of a hyaluronan operably linked to a triple promoter comprising a variant amyL promoter having a mutation corresponding to position 590 of SEQ ID NO: 1, a consensus promoter having the sequence TTGACA for the “−35” region and TATAAT for the “−10” region, and a cryIIIA promoter, in which each promoter sequence of the triple promoter is operably linked to one or more coding sequences involved in the biosynthesis of the hyaluronic acid. The promoter sequences of the triple promoter may be in any order.

In the methods of the present invention, the components of the triple promoter can be obtained from any bacterial source. In a preferred aspect, the promoter sequences are obtained from a gram positive bacterium such as a Bacillus strain, e.g., Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis; or a Streptomyces strain, e.g., Streptomyces lividans or Streptomyces murinus; or from a gram negative bacterium, e.g., E. coli or Pseudomonas sp.

An example of a suitable amyL promoter for use in the methods of the present invention is the promoter of the Bacillus licheniformis alpha-amylase gene (amyL). An example of a suitable cryIIIA promoter for use in the methods of the present invention is the promoter of the Bacillus thuringiensis subsp. tenebrionis cryIIIA gene.

In the methods of the present invention, a variant amyL promoter having a mutation corresponding to position 590 of SEQ ID NO: 1, where T was changed to A at position 590 to produce SEQ ID NO: 1, can be obtained according to U.S. Pat. Nos. 5,698,415 and 6,100,063. U.S. Pat. No. 5,698,415 claims variant promoters derived from the Bacillus licheniformis amyL promoter. With reference to claim 1 in the above patent, such a variant promoter is a fragment of the sequence given in this claim, in which N2-N9 has the sequence ATGTATCA. Such a variant promoter is constructed by incorporating the desired mutation into a long PCR primer, 28902 (U.S. Pat. No. 6,100,063), covering the amyL promoter region. Another PCR primer, LWN3216 (U.S. Pat. No. 6,100,063), reads upstream from a position spanning the PstI site in the AmyL signal peptide coding region. Together, these primers allow PCR amplification of a variant amyL promoter fragment derived from a parent amyL promoter.

In the present invention, the parent amyL promoter is (a) a polynucleotide having a nucleotide sequence which has at least 70% identity with SEQ ID NO: 1; or (b) a polynucleotide having a nucleotide sequence which hybridizes under at least low stringency conditions with SEQ ID NO: 1, or its complementary strand.

In a first aspect, the parent amyL promoter comprises a nucleotide sequence which has a degree of identity to SEQ ID NO: 1 of at least 70%, preferably at least 75%, more preferably at least 80%, more preferably at least 85%, even more preferably at least 90%, most preferably at least 95%, and even most preferably at least 97% (hereinafter “homologous amyL promoters”).

Preferably, the parent amyL promoter comprises the nucleotide sequence of SEQ ID NO: 1; or a fragment thereof that has promoter activity. In a preferred embodiment, the parent amyl promoter comprises the nucleotide sequence of SEQ ID NO: 1. In another preferred embodiment, the parent amyL promoter consists of the nucleotide sequence of SEQ ID NO: 1.

In a second aspect, the parent amyL promoter is a nucleotide sequence which hybridizes under low stringency conditions, preferably medium stringency conditions, more preferably medium-high stringency conditions, even more preferably high stringency conditions, and most preferably very high stringency conditions with SEQ ID NO: 1 or its complementary strand (J. Sambrook, E. F. Fritsch, and T. Maniatus, 1989, Molecular Cloning, A Laboratory Manual, 2d edition, Cold Spring Harbor, N.Y.). Such stringency conditions are defined herein.

The nucleotide sequence of SEQ ID NO: 1 or a fragment thereof may be used to identify and clone homologous amyL promoters from strains of different genera or species according to methods well known in the art.

In the present invention, an isolated amyL promoter variant comprises a nucleotide sequence which has a degree of identity of at least 70%, preferably at least 75%, more preferably at least 80%, more preferably at least 85%, even more preferably at least 90%, most preferably at least 95%, and even most preferably at least 97% to SEQ ID NO: 1.

For purposes of the present invention, the degree of identity between two nucleotide sequences is determined by the Wilbur-Lipman method (Wilbur and Lipman, 1983, Proceedings of the National Academy of Science USA 80: 726-730) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

The construction of a “consensus” promoter may be accomplished by site-directed mutagenesis to create a promoter which conforms more perfectly to the established consensus sequences for the “−10” and “−35” regions of the vegetative “sigma A-type” promoters for Bacillus subtilis (Voskuil et al., 1995, Molecular Microbiology 17: 271-279). The consensus sequence for the “−35” region is TTGACA and for the “−10” region is TATAAT. The consensus promoter may be obtained from any promoter which can function in a Bacillus host cell.

In a preferred aspect, the “consensus” promoter is obtained from a promoter obtained from the E. coli lac operon, Streptomyces coelicolor agarase gene (dagA), Bacillus lentus alkaline protease gene (aprH), Bacillus licheniformis alkaline protease gene (subtilisin Carlsberg gene), Bacillus subtilis levansucrase gene (sacB), Bacillus subtilis alpha-amylase gene (amyE), Bacillus licheniformis alpha-amylase gene (amyL), Bacillus stearothermophilus maltogenic amylase gene (amyM), Bacillus amyloliquefaciens alpha-amylase gene (amyQ), Bacillus lichenifomis penicillinase gene (penP), Bacillus subtilis xyIA and xyIB genes, Bacillus thuringiensis subsp. tenebrionis cryIIIA gene (SEQ ID NO: 41) or portions thereof, or prokaryotic beta-lactamase gene. The “consensus” promoter can also be obtained from the spo1 bacterial phage promoter.

In a more preferred aspect, the “consensus” promoter is obtained from a Bacillus amyloliquefaciens alpha-amylase gene (amyQ). In a most preferred aspect, the consensus promoter is the “consensus” amyQ promoter contained in nucleotides 1 to 185 of SEQ ID NO: 42 or SEQ ID NO: 43. In another most preferred aspect, the consensus promoter is the short “consensus” amyQ promoter contained in nucleotides 86 to 185 of SEQ ID NO: 42 or SEQ ID NO: 43. The “consensus” amyQ promoter of SEQ ID NO: 42 contains the following mutations of the nucleotide sequence containing the wild-type amyQ promoter (SEQ ID NO: 44): T to A and T to C in the −35 region (with respect to the transcription start site) at positions 135 and 136, respectively, and an A to T change in the −10 region at position 156 of SEQ ID NO: 44. The “consensus” amyQ promoter of SEQ ID NO: 43 further contains a T to A change at position 116, approximately 20 base pairs upstream of the −35 region (SEQ ID NO: 43), where the change has apparently no detrimental effect on promoter function since it is well removed from the critical −10 and −35 regions.

In a preferred aspect, the triple promoter comprises a variant amyL promoter having a mutation corresponding to position 590 of SEQ ID NO: 1. In another preferred aspect, the triple promoter comprises the variant amyL promoter of SEQ ID NO: 1. In another preferred aspect, the triple promoter comprises a consensus amyQ promoter having the sequence TTGACA for the “−35” region and TATAAT for the “−10” region. In another preferred aspect, the triple promoter comprises a short consensus amyQ promoter having the sequence TTGACA for the “−35” region and TATAAT for the “−10” region. In another preferred aspect, the triple promoter comprises the cryIIIA promoter or a portion thereof (Agaisse and Lereclus, 1994, Molecular Microbiology 13: 97-107).

In a more preferred aspect, the triple promoter comprises a variant amyL promoter having a mutation corresponding to position 590 of SEQ ID NO: 1, a short consensus amyQ promoter having the sequence TTGACA for the “−35” region and TATAAT for the “−10” region, and a cryIIIA promoter.

In another more preferred aspect, the triple promoter comprises a variant amyL promoter having a mutation corresponding to position 590 of SEQ ID NO: 1, a short consensus amyQ promoter having the sequence TTGACA for the “−35” region and TATAAT for the “−10” region, and a cryIIIA promoter, in the above order 5′ to 3′.

In most preferred aspect, the triple promoter comprises the variant amyL promoter of SEQ ID NO: 1, a short consensus amyQ promoter having the sequence TTGACA for the “−35” region and TATAAT for the “−10” region, and a cryIIIA promoter of SEQ ID NO: 41.

In another most preferred aspect, the triple promoter comprises the variant amyL promoter of SEQ ID NO: 1, a short consensus amyQ promoter having the sequence TTGACA for the “−35” region and TATAAT for the “−10” region, and a cryIIIA promoter of SEQ ID NO: 41, in the above order 5′ to 3′.

“An mRNA processing/stabilizing sequence” is defined herein as a sequence located downstream of one or more promoter sequences of the triple promoter and upstream of one or more coding sequences to which each of the triple promoter sequences are operably linked such that all mRNAs synthesized from the one or more promoter sequences may be processed to generate mRNA transcripts with a stabilizer sequence at the 5′ end of the transcripts. The presence of such a stabilizer sequence at the 5′ end of the mRNA transcripts increases their half-life (Agaisse and Lereclus, 1994, supra, Hue et al., 1995, Journal of Bacteriology 177: 3465-3471). The mRNA processing/stabilizing sequence is complementary to the 3′ extremity of a bacterial 16S ribosomal RNA. In a preferred aspect, the mRNA processing/stabilizing sequence generates essentially single-size transcripts with a stabilizing sequence at the 5′ end of the transcripts. In another preferred aspect, the mRNA processing/stabilizing sequence is located downstream of the entire triple promoter and upstream of the one or more coding sequences. In another preferred aspect, the mRNA processing/stabilizing sequence is located downstream of the cryIIIA promoter of the triple promoter and upstream of the one or more coding sequences.

The mRNA processing/stabilizing sequence is preferably located downstream of the triple promoter and upstream of the one or more coding sequences involved in the biosynthesis of the hyaluronic acid. However, the mRNA processing/stabilizing sequence can be located downstream of any of the promoter sequences of the triple promoter and upstream of the one or more coding sequences involved in the biosynthesis of the hyaluronic acid. Furthermore, an mRNA processing/stabilizing sequence can be located downstream of each of the promoter sequences of the triple promoter and upstream of the one or more coding sequences involved in the biosynthesis of the hyaluronic acid. The mRNA processing stabilizing sequence or sequences may be foreign to one or more of the promoter sequences of the triple promoter and/or foreign to each other.

In a preferred aspect, the mRNA processing/stabilizing sequence is the Bacillus thuringiensis cryIIIA mRNA processing/stabilizing sequence disclosed in WO 94/25612 and Agaisse and Lereclus, 1994, supra, or portions thereof which retain the mRNA processing/stabilizing function. In another more preferred aspect, the mRNA processing/stabilizing sequence is the Bacillus subtilis SP82 mRNA processing/stabilizing sequence disclosed in Hue et al., 1995, supra, or portions thereof which retain the mRNA processing/stabilizing function.

When the cryIIIA promoter and its mRNA processing/stabilizing sequence are employed in the methods of the present invention, a DNA fragment containing the sequence disclosed in WO 94/25612 and Agaisse and Lereclus, 1994, supra, delineated by nucleotides −635 to −22 of SEQ ID NO: 41, or portions thereof which retain the promoter and mRNA processing/stabilizing functions, may be used. The cryIIIA promoter is delineated by nucleotides −635 to −552 while the cryIIIA mRNA processing/stabilizing sequence is contained within nucleotides −551 to −22. In a preferred aspect, the cryIIIA mRNA processing/stabilizing sequence is contained in a fragment comprising nucleotides −568 to −22. In another preferred aspect, the cryIIIA mRNA processing/stabilizing sequence is contained in a fragment comprising nucleotides −367 to −21. Furthermore, DNA fragments containing only the cryIIIA promoter and/or only the cryIIIA mRNA processing/stabilizing sequence may be prepared using methods well known in the art to construct various triple promoter and mRNA processing/stabilizing sequence combinations.

In a preferred aspect, the cryIIIA promoter and its mRNA processing/stabilizing sequence are preferably placed downstream of the other promoter sequences constituting the triple promoter and upstream of the one or more coding sequences.

In a more preferred aspect, the triple promoter comprises a variant amyL promoter having a mutation corresponding to position 590 of SEQ ID NO: 1, a short consensus amyQ promoter having the sequence TTGACA for the “−35” region and TATAAT for the “−10” region, wherein the promoter sequences are in any order, and a cryIIIA promoter, and the cryIIIA mRNA processing/stabilizing sequence.

In another more preferred aspect, the triple promoter comprises the variant amyl promoter of SEQ ID NO: 1, a short consensus amyQ promoter having the sequence TTGACA for the “−35” region and TATAAT for the “−10” region, wherein the promoter sequences are in any order, and a cryIIIA promoter of SEQ ID NO: 41, and the cryIIIA mRNA processing/stabilizing sequence.

In a most preferred aspect, the triple promoter comprises a variant amyL promoter having a mutation corresponding to position 590 of SEQ ID NO: 1, a short consensus amyQ promoter having the sequence TTGACA for the “−35” region and TATAAT for the “−10” region, and a cryIIIA promoter, and the cryIIIA mRNA processing/stabilizing sequence, in the above order 5′ to 3′.

In another most preferred aspect, the triple promoter comprises the variant amyl promoter of SEQ ID NO: 1, a short consensus amyQ promoter having the sequence TTGACA for the “−35” region and TATAAT for the “−10” region, and a cryIIIA promoter of SEQ ID NO: 41, and the cryIIIA mRNA processing/stabilizing sequence, in the above order 5′ to 3′.

The one or more coding sequences involved in the biosynthesis of a hyaluronic acid may be further manipulated by operably linking the one or more coding sequences to one or more additional control sequences which direct the expression of the coding sequence in a Bacillus cell under conditions compatible with the control sequences. Expression will be understood to include any step involved in the production of the polypeptide including, but not limited to, transcription, post-transcriptional modification, translation, post-translational modification, and translocation. The techniques for modifying nucleotide sequences utilizing cloning methods are well known in the art.

The term “control sequences” is defined herein to include all components which are necessary or advantageous for expression of a coding sequence. Each control sequence may be native or foreign to the one or more coding sequences. In addition to the triple promoter, described earlier, such control sequences include, but are not limited to, a leader, a signal sequence, and a transcription terminator. The control sequences may be provided with linkers for the purpose of introducing specific restriction sites facilitating ligation of the control sequences with the coding regions.

The control sequence may also be a suitable transcription terminator sequence, a sequence recognized by a Bacillus cell to terminate transcription. The terminator sequence is operably linked to the 3′ terminus of the polynucleotide encoding the polypeptide. Any terminator which is functional in the Bacillus cell of choice may be used in the present invention.

The control sequence may also be a suitable leader sequence, a nontranslated region of an mRNA which is important for translation by the Bacillus cell. The leader sequence is operably linked to the 5′ terminus of the polynucleotide encoding the polypeptide. Any leader sequence which is functional in the Bacillus cell of choice may be used in the present invention.

The control sequence may also be a signal peptide coding region that codes for an amino acid sequence linked to the amino terminus of the polypeptide which translocates the expressed polypeptide, for example, into the cell's membrane. The signal peptide coding region may be native to the polypeptide or may be obtained from foreign sources. The 5′ end of the coding sequence may inherently contain a signal peptide coding region naturally linked in translation reading frame with the segment of the coding region which encodes the translocated polypeptide. Alternatively, the 5′ end of the coding sequence may contain a signal peptide coding region which is foreign to that portion of the coding sequence which encodes the transocated polypeptide. The foreign signal peptide coding region may be required where the coding sequence does not normally contain a signal peptide coding region. Alternatively, the foreign signal peptide coding region may simply replace the natural signal peptide coding region in order to obtain enhanced translocation of the polypeptide relative to the natural signal peptide coding region normally associated with the coding sequence. Any signal peptide coding region capable of directing translocation of the expressed polypeptide may be used in the present invention.

The Bacillus cell may contain one or more copies of at least two different nucleic acid constructs, wherein each of the constructs are constructed as described supra.

A nucleic acid construct may further contain one or more selectable markers which permit easy selection of transformed cells. A selectable marker is a gene the product of which provides for biocide resistance, resistance to heavy metals, prototrophy to auxotrophs, and the like. Examples of bacterial selectable markers are the dal genes from Bacillus subtilis or Bacillus licheniformis, or markers which confer antibiotic resistance, such as ampicillin, kanamycin, erythromycin, chloramphenicol or tetracycline resistance. Furthermore, selection may be accomplished by co-transformation, e.g., as described in WO 91/09129, where the selectable marker is on a separate vector.

A particular advantage of the present invention is that a Bacillus cell can be produced free of a foreign selectable marker gene, i.e., after the introduction of the nucleic acid construct into the Bacillus cell, the foreign selectable marker gene can be deleted from the Bacillus cell making the cell marker-free. Removal of the selectable marker gene to produce a Bacillus cell free of such a marker may be preferable for regulatory and environmental reasons.

Gene deletion or replacement techniques may be used for the complete removal of the selectable marker gene. For example, the deletion of the selectable marker gene may be accomplished by homologous recombination using a plasmid which has been constructed to contiguously contain the 5′ and 3′ regions flanking the selectable marker gene. The contiguous 5′ and 3′ regions may be introduced into a Bacillus cell on a temperature-sensitive plasmid, e.g., pE194, in association with a second selectable marker at a permissive temperature to allow the plasmid to become established in the cell. The cell is then shifted to a non-permissive temperature to select for cells which have the plasmid integrated into the chromosome at one of the homologous flanking regions. Selection for integration of the plasmid is effected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers (see, for example, Perego, 1993s, In A. L. Sonneshein, J. A. Hoch, and R. Losick, editors, Bacillus subtilis and Other Gram-Positive Bacteria, Chapter 42, American Society of Microbiology, Washington, D.C., 1993). Other methods well known in the art may also be used.

Expression Vectors

In the methods of the present invention, a recombinant expression vector comprising one or more genes involved in the biosynthesis of a hyaluronic acid, a triple promoter and, alternatively also, an mRNA processing/stabilizing sequence, and transcriptional and translational stop signals may be used for recombinant production. The various nucleic acid and control sequences described above may be joined together to produce a recombinant expression vector which may include one or more convenient restriction sites to allow for insertion or substitution of the polynucleotide at such sites. Alternatively, the one or more genes involved in the biosynthesis of a hyaluronic acid may be expressed by inserting the gene or genes or a nucleic acid construct comprising the one or more genes into an appropriate vector for expression. In creating the expression vector, the one or more genes involved in the biosynthesis of a hyaluronic acid are located in the vector so that the coding sequence or sequences are operably linked with a triple promoter and alternatively also an mRNA processing/stabilizing sequence, and any other appropriate control sequences for expression, and possibly translocation.

The recombinant expression vector may be any vector which can be conveniently subjected to recombinant DNA procedures and can bring about the expression of the one or more genes involved in the biosynthesis of a hyaluronic acid on introduction into a Bacillus cell. The choice of the vector will typically depend on the compatibility of the vector with the Bacillus cell into which the vector is to be introduced. The vectors may be linear or closed circular plasmids. The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the Bacillus cell, is integrated into the chromosome and replicated together with the chromosome into which it has been integrated. The vector system may be a single vector or plasmid or two or more vectors or plasmids, or a transposon.

“Introduction” means introducing a vector comprising the one or more genes involved in the biosynthesis of a hyaluronic acid into a Bacillus cell so that the vector is maintained as a chromosomal integrant or as a self-replicating extrachromosomal vector. Integration is generally considered to be an advantage as the one or more coding sequences or genes are more likely to be stably maintained in the cell. Integration of the vector into the chromosome occurs by homologous recombination, non-homologous recombination, or transposition.

The introduction of an expression vector into a Bacillus cell may, for instance, be effected by protoplast transformation, using competent cells, electroporation, or conjugation, as described herein.

For integration, the vector may rely on any component of the vector for stable integration of the vector into the genome by homologous recombination. The vector may contain additional polynucleotide sequences for directing integration by homologous recombination into the genome of the Bacillus cell. The additional polynucleotide sequences enable the vector to be integrated into the Bacillus cell genome at a precise location in the chromosome. To increase the likelihood of integration at a precise location, the integrational components should preferably contain a sufficient number of nucleic acids, such as 100 to 10,000 base pairs, preferably 400 to 10,000 base pairs, and most preferably 800 to 10,000 base pairs, which are highly homologous with the corresponding target sequence to enhance the probability of homologous recombination. The integrational components may be any polynucleotide sequence that is homologous with the target sequence in the genome of the Bacillus cell. Furthermore, the integrational components may be non-encoding or encoding sequences.

For autonomous replication, the vector may further comprise an origin of replication enabling the vector to replicate autonomously in the Bacillus cell in question. Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus. The origin of replication may be one having a mutation to make its function temperature-sensitive in the Bacillus cell (see, e.g., Ehrlich, 1978, Proceedings of the National Academy of Sciences USA 75:1433).

The procedures used to ligate the components described above to construct the recombinant expression vectors are well known to one skilled in the art (see, e.g., Sambrook et al., 1989, supra).

Production

In the methods of the present invention, the Bacillus host cells are cultivated in a nutrient medium suitable for production of hyaluronic acid using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the enzymes involved in hyaluronic acid synthesis to be expressed and the hyaluronic acid to be isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). The secreted hyaluronic acid can be recovered directly from the medium.

The resulting hyaluronic acid may be isolated using methods well known in the art. For example, the hyaluronic acid may be isolated from the nutrient medium by conventional procedures including, but not limited to, centrifugation, filtration, extraction, spray-drying, evaporation, or precipitation. The isolated hyaluronic acid may then be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), or extraction (see, e.g., Protein Purification, J.-C. Janson and Lars Ryden, editors, VCH Publishers, New York, 1989).

Since the hyaluronan of the recombinant Bacillus cell is expressed directly into the culture medium, a simple process may be used to isolate the hyaluronan from the culture medium. First, the Bacillus cells and cellular debris are physically removed from the culture medium. The culture medium may be diluted first, if desired, to reduce the viscosity of the medium. Many methods are known to those skilled in the art for removing cells from culture medium, such as centrifugation or microfiltration. If desired, the remaining supernatant may then be filtered, such as by ultrafiltration, to concentrate and remove small molecule contaminants from the hyaluronan. Following removal of the cells and cellular debris, a simple precipitation of the hyaluronan from the medium is performed by known mechanisms. Salt, alcohol, or combinations of salt and alcohol may be used to precipitate the hyaluronan from the filtrate. Once reduced to a precipitate, the hyaluronan can be easily isolated from the solution by physical means. Alternatively, the hyaluronan may be dried or concentrated from the filtrate solution by using evaporative techniques known to the art, such as spray drying. The level of hyaluronic acid produced by a Bacillus host cell of the present invention may be determined according to the modified carbazole method as describe herein.

In the methods of the present invention, the Bacillus cell preferably produces at least 25% more, more preferably at least 50% more, more preferably at least 75% more, more preferably at least 100% more, even more preferably at least 200% more, most preferably at least 300% more, and even most preferably at least 400% more hyaluronic acid relative to a Bacillus cell containing only one of the promoter sequences of the triple promoter operably linked to one or more coding sequences involved in the biosynthesis of the hyaluronic acid when cultured under identical production conditions.

Deletions/Disruptions

Gene deletion or replacement techniques may be used for the complete removal of a foreign or heterologous selectable marker gene or other undesirable gene. In such methods, the deletion of the selectable marker gene may be accomplished by homologous recombination using a plasmid that has been constructed to contiguously contain the 5′ and 3′ regions flanking the selectable marker gene. For example, the contiguous 5′ and 3′ regions may be introduced into a Bacillus cell on a temperature-sensitive plasmid, e.g., pE194, in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is effected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers (see, for example, Perego, 1993, In A. L. Sonneshein, J. A. Hoch, and R. Losick, editors, Bacillus subtilis and Other Gram-Positive Bacteria, Chapter 42, American Society of Microbiology, Washington, D.C., 1993).

A selectable marker gene may also be removed by homologous recombination by introducing into the mutant cell a nucleic acid fragment comprising 5′ and 3′ regions of the defective gene, but lacking the selectable marker gene, followed by selecting on the counter-selection medium. By homologous recombination, the defective gene containing the selectable marker gene is replaced with the nucleic acid fragment lacking the selectable marker gene. Other methods known in the art may also be used.

The procedures described above can also be used to delete or disrupt an undesirable gene. U.S. Pat. No. 5,891,701 discloses techniques for deleting several genes including spoIIAC, aprE, nprE, and amyE.

Other undesirable biological compounds may also be removed by the above described methods such as the red pigment synthesized by cypX (accession no. BG12580) and/or yvmC (accession no. BG14121).

In a preferred aspect, the Bacillus host cell is unmarked with any foreign or heterologous selectable markers. In another preferred aspect, the Bacillus host cell does not produce any red pigment synthesized by cypX and yvmC.

The present invention is further described by the following examples which should not be construed as limiting the scope of the invention.

### EXAMPLES

Primers and Oligos

All primers and oligos were synthesized on an Applied Biosystems Model 394 Synthesizer (Applied Biosystems, Inc., Foster City, Calif.) according to the manufacturer's instructions.

### Example 1

### Construction of Bacillus licheniformis MaTa2 Harboring the P11 Promoter/amyL Expression Cassette in the amyL Locus

The P11 promoter (Pr_{short “consensus” amyQ}/Pr_{cryIIIA stab}, U.S. Pat. No. 6,255,076) was introduced upstream of the amyL gene (essentially identical to accession no. M13256, Gray et al., 1986, J. Bacteriol. 166: 635, except there is a C at position 474 in place of T, a G at position 523 in place of C, a C at position 524 in place of G, and AAA at positions 768-770 in place of TTT) in Bacillus licheniformis SJ1904 (U.S. Pat. No. 5,733,753) by standard gene replacement procedures. Because of difficulties working with plasmids bearing fragments of the amyL coding region located downstream of functional promoters, the construction was performed in two steps as described below. First the amyL promoter and amyL ribosome-binding site (RBS) were replaced with a composite promoter lacking the RBS (to prevent expression of amyL). A second gene replacement was then performed to reintroduce the RBS thereby generating a functional expression cassette.

The pE194-based, temperature-sensitive plasmid pMRT064.1 (WO 03/054163) was introduced into Bacillus licheniformis strain SJ1904 via electroporation (Xue et al., 1999, Journal of Microbiological Methods 34: 183-191). The cells were plated onto Tryptose blood agar base (TBAB)-agar plates supplemented with 1 μg of erythromycin and 25 μg of lincomycin per ml after incubation at 28° C. for 24-48 hours. TBAB agar plates were composed per liter of 33 g of Tryptose blood agar base. An erythromycin-resistant transformant was isolated and grown in the presence of erythromycin (5 μg/ml) at the non-permissive temperature of 50° C. At this temperature, the pE194 origin of replication is inactive. Cells were able to grow in the presence of erythromycin only by integration of the plasmid into the amyL locus of the bacterial chromosome. To promote the loss or “looping out” of the plasmid, which would result in the replacement of the endogenous amyL promoter with the P11 promoter and the subsequent loss of the RBS, the integrants were grown in Luria-Bertani (LB) medium without selection at the permissive temperature of 30° C. for several generations. LB medium was composed per liter of 10 g of tryptone, 5 g of yeast extract, and 5 g of NaCl. At this temperature, the pE194 origin of replication was active and promoted excision of the plasmid from the genome (Molecular Biological Methods for Bacillus, edited by C. R. Harwood and S. M. Cutting, 1990, John Wiley and Sons Ltd.). The cells were then plated on non-selective LB agar plates (LB medium plus 15 g of Bacto agar per liter of LB medium) and colonies which contained the desired promoter replacement and loss of the pE194-based replicon were identified by the following criteria: (1) the inability to form halos on TBAB plates containing a 0.5% starch-azure (Sigma Chemical Co., St. Louis, Mo.) plus TBAB overlay indicated the presence of the P11 promoter and the loss of the amyL RBS; and (2) erythromycin sensitivity indicated the loss of the pE194-based replicon. One transformant was chosen, which met these three criteria, and was designated Bacillus licheniformis SJ1904::pMRT064.1.

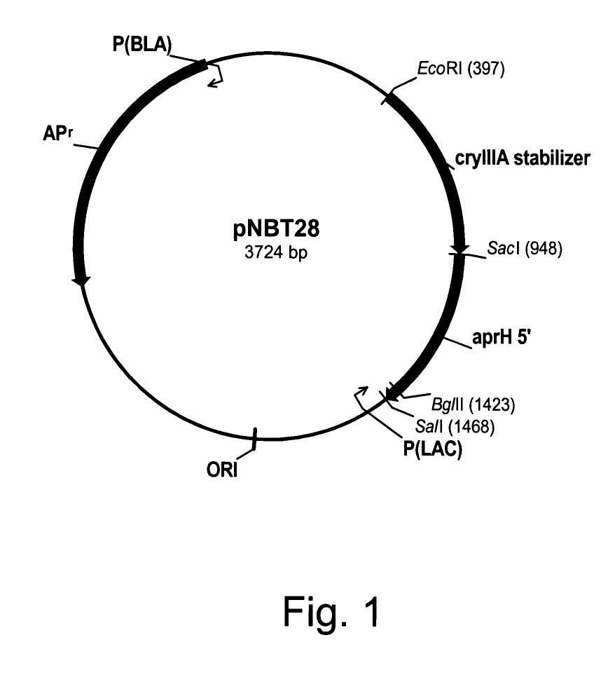

The amyL RBS was restored in the above strain as follows. Plasmid pNBT23 (pDG268MCSΔneo-Pr_{short “consensus” amyQ}/Pr_{cryIIIA}/cryIIIAstab/SAV, U.S. Pat. No. 6,255,076) was digested with PacI, the ends were blunted with T4 DNA polymerase I (Roche Applied Science, Indianapolis, Ind.), and then digested with SalI. Plasmid pUC19 (Yanisch-Perron et al., 1985, Gene 33: 103-119) was digested with Ecl13611 and SalI. The digestions were resolved on a 0.8% agarose gel using 44 mM Tris Base, 44 mM boric acid, 0.5 mM EDTA (0.5×TBE) buffer and the larger vector fragment (approximately 2661 bp) from pUC19 and the smaller cryIIIAstab/aprH 5′ fragment (approximately 1069 bp) from pNBT23 were gel purified using a QIAquick DNA Extraction Kit according to the manufacturer's instructions (QIAGEN Inc., Valencia, Calif.). The two purified fragments were ligated together with T4 DNA ligase according to the manufacturer's instructions (Roche Applied Science; Indianapolis, Ind.) and the ligation mix was transformed into E. coli SURE® competent cells (Stratagene, Inc., La Jolla, Calif.). Transformants were selected on 2×yeast-tryptone (2×YT) agar plates supplemented with 100 μg of ampicillin per ml. 2×YT plates were composed per liter of 16 g of tryptone, 10 g of yeast extract, 5 g of NaCl, and 15 g of Bacto agar. Plasmid DNA was purified from several transformants using a Bio Robot 9600 according to the manufacturer's instructions (QIAGEN Inc., Valencia, Calif.) and analyzed by EcoRI plus SalI digestion on a 0.8% agarose gel using 0.5× TBE buffer. The correct plasmid was identified by the presence of an approximately 1071 bp EcoRI/SalI cryIIIAstab/aprH 5′ fragment and was designated pNBT28 (FIG. 1).

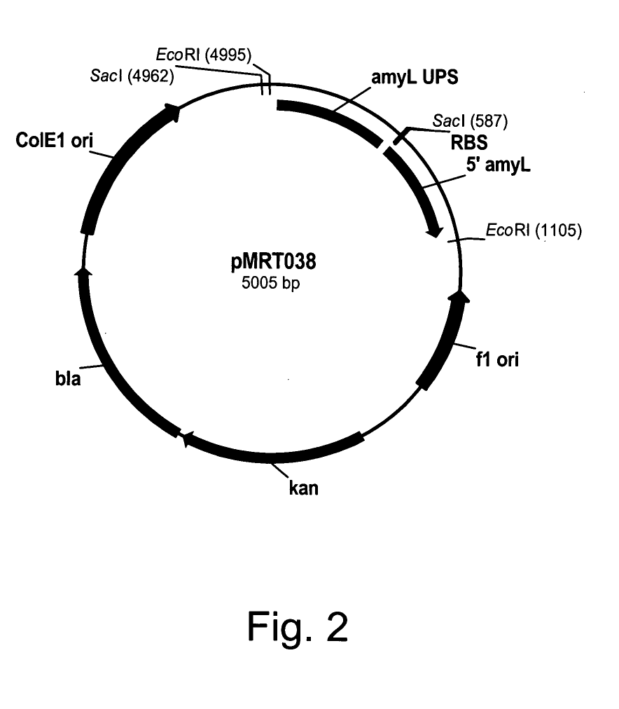

Plasmid pMRT038 was constructed by splicing by overlap extension (SOE) according to the procedure of Horton et al., 1989, Gene 77: 61-8. The amyL promoter region and 5′ coding sequence from plasmid pDN1981 (U.S. Pat. No. 5,698,415) were PCR amplified using primers pairs 733-45-1 and 733-45-2, and 733-68-1 and 733-70-1, respectively, as shown below. PCR amplification was conducted in 50 μl reactions composed of 1 ng of pDN1981 DNA, 0.4 μM of each primer, 200 μM each of dATP, dCTP, dGTP, and dTTP, 1×PCR Buffer II (Applied Biosystems, Inc., Foster City, Calif.) with 2.5 mM MgCl₂, and 2.5 units of AmpliTaq Gold™ DNA polymerase (Applied Biosystems, Inc., Foster City, Calif.). The reactions were performed in a RoboCycler 40 thermacycler (Stratagene, Inc., La Jolla, Calif.) programmed for 1 cycle at 95° C. for 10 minutes; 25 cycles each at 95° C. for 1 minute, 50° C. for 1 minute, and 72° C. for 1 minute; and 1 cycle at 72° C. for 7 minutes. The PCR product was visualized using a 0.8% agarose gel with 0.5×TBE buffer. The expected fragments for the amyL promoter region and 5′ coding sequence were approximately 600 and 500 bp, respectively. The final SOE fragment was amplified using primers 733-45-1 and 733-70-1. The final SOE fragment was cloned into pCR2.1 using a TA-TOPO Cloning Kit (Stratagene, Inc., La Jolla, Calif.). Transformants were selected on 2×YT agar plates supplemented with 100 μg of ampicillin per ml and incubated at 37° C. for 16 hours. Transformants carrying the correct plasmid were verified by DNA sequencing using M13 forward and reverse primers (Invitrogen, Inc, Carlsbad, Calif.). This plasmid was designated pMRT038 (FIG. 2).

Primer 733-45-1:

| | |
|---|---|
| 5′-GTCCTTCTTGGTACCTGGAAGCAGAGC-3′ | (SEQ ID NO: 45) |
| Primer 733-45-2: | |
| 5′GTATAAATATTCGGCCCTTAAGGCCAGTACCATTTTCCC-3′ | (SEQ ID NO: 46) |
| Primer 733-68-1: | |
| 5′-TGGTACTGGCCTTAAGGGCCGAATATTTATACAATATCATGAGCTCCACATTGAA | (SEQ ID NO: 47) |
| AGGG-3′ | |
| Primer 733-70-1: | |
| 5′-GGTGTTCTCTAGAGCGGCCGCGGTTGCGGTCAGC-3′ | (SEQ ID NO: 48) |

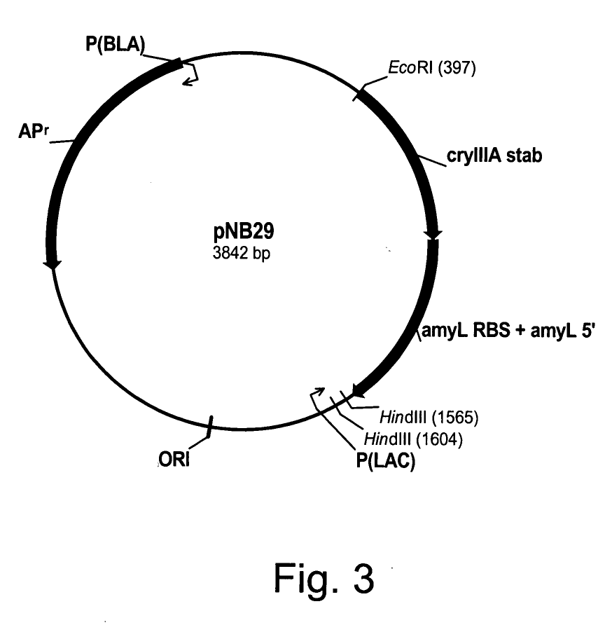

Plasmid pNBT28 was digested with BglII, the ends were blunted with Klenow fragment, and then digested with SacI. Plasmid pMRT038 was digested with EcoRI, the ends were blunted with Klenow fragment, and then digested with SacI. The digestions were resolved on a 0.8% agarose gel using 0.5×TBE buffer and the larger vector fragment (approximately 3253 bp) from pNBT28 and the smaller fragment (approximately 593 bp) from pMRT038 were gel purified using a QIAquick DNA Extraction Kit according to the manufacturer's instructions. The two purified fragments were ligated together with T4 DNA ligase and the ligation mix was transformed into E. coli SURE® competent cells. Transformants were selected on 2×YT agar plates supplemented with 100 μg of ampicillin per ml. Plasmid DNA was purified from several transformants using a QIAGEN Bio Robot 9600 according to the manufacturer's instructions and analyzed by EcoRI plus HindIII digestion on a 0.8% agarose gel using 0.5×TBE buffer. The correct plasmid was identified by the presence of an approximately 1168 bp EcoRI/HindIII fragment and was designated pNBT29 (FIG. 3).

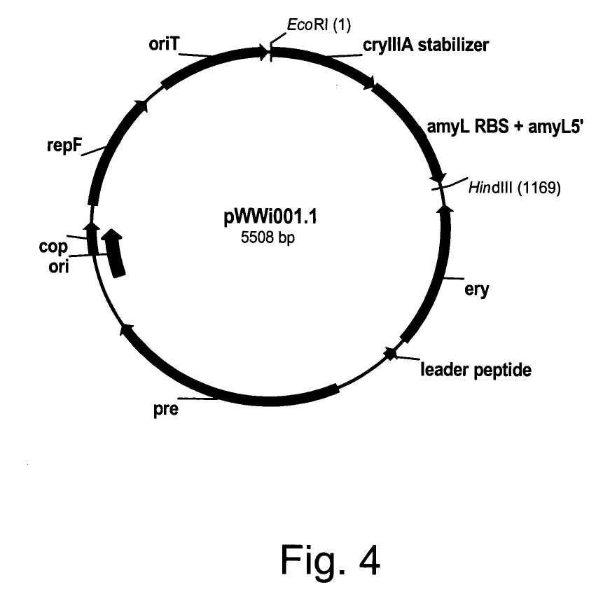

Plasmids pNBT29 and pCJ791 (WO 03/054163) were digested with EcoRI and HindIII. The digestions were resolved on a 0.8% agarose gel using 0.5×TBE buffer and the larger vector fragment (approximately 4340 bp) from pCJ791 and the smaller fragment (approximately 1168 bp) from pNBT29 were gel purified using a QIAquick DNA Extraction Kit according to the manufacturer's instructions. The two purified fragments were ligated together with T4 DNA ligase and the ligation mix was transformed into Bacillus subtilis 168Δ4 (WO 03/054163). Plasmid DNA was purified from several transformants using tip-20 columns (QIAGEN Inc., Valencia, Calif.) according to the manufacturer's instructions and analyzed by EcoRI plus HindIII digestion on a 0.8% agarose gel using 0.5×TBE buffer. The correct plasmid was identified by the presence of an approximately 1168 bp EcoRI/HindIII fragment and was designated pWWi001.1 (FIG. 4).

In order to restore the amyL RBS in Bacillus licheniformis strain SJ1904::pMRT064.1, plasmid pWWi001.1 was introduced into this strain via electroporation, then integrated into the chromosome and excised as described in Example 1, creating Bacillus licheniformis MaTa2. The desired clone was identified by the restoration of amyL expression which was assayed by growing the strain on TBAB plates containing a 0.5% starch-azure plus TBAB overlay. Strains producing amylase formed a clear halo around the colony or patch.

### Example 2

### Construction of Bacillus licheniformis MaTa3 Harboring the P12 Promoter/amyL Expression Cassette in the Native amyL Locus

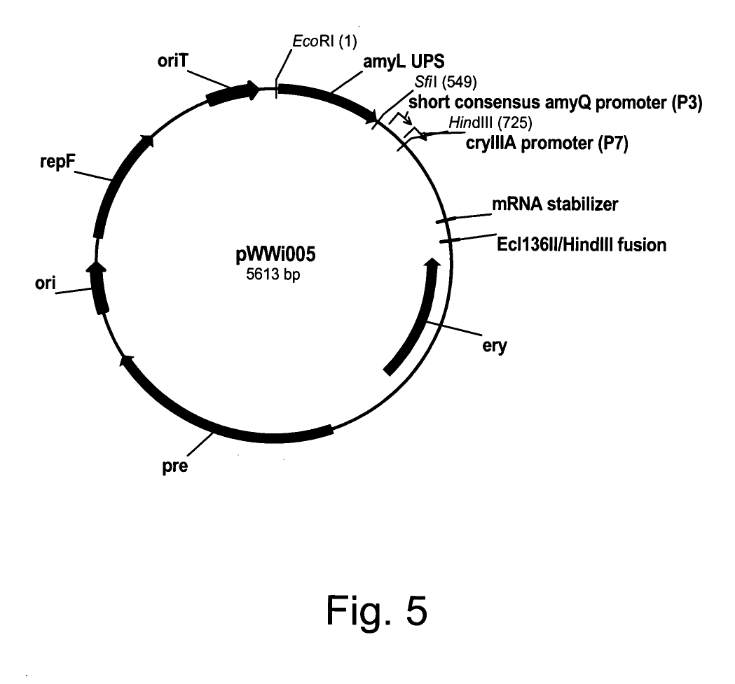

Plasmid pMRT064.1 was digested with HindIII, filled-in with Klenow fragment, and then digested with Sfil. Plasmid pNBT23 (U.S. Pat. No. 6,255,076) was digested with SfiI and Ecl136II. The digestions were resolved on a 0.8% agarose gel using 0.5×TBE buffer and the larger vector fragment (approximately 4892 bp) from pMRT064.1 and the smaller fragment (approximately 728 bp) from pNBT23 were gel purified using a QIAquick DNA Extraction Kit according to the manufacturer's instructions. The two purified fragments were ligated together with T4 DNA ligase and the ligation mix was transformed into Bacillus subtilis 168Δ4. Plasmid DNA was purified from several transformants using QIAGEN tip-20 columns according to the manufacturer's instructions and analyzed by EcoRI plus HindIII digestion on a 0.8% agarose gel using 0.5×TBE buffer. The correct plasmid was verified by restriction enzyme and/or PCR analysis and was designated pWWi005 (FIG. 5).

Plasmid pWWi005 was introduced into Bacillus licheniformis MaTa2 via electroporation, then integrated and excised from the chromosome as described in Example 1, replacing the P11 promoter with the P12 promoter (Pr_{short “consensus” amyQ}/Pr_{cryIIIA}/cryIIIAstab, U.S. Pat. No. 6,255,076), to create Bacillus licheniformis strain MaTa3. The desired clone was identified by PCR analysis using primers 961197 and 94-935 shown below. PCR amplification was conducted in 50 μl reactions composed of 200 ng of Bacillus licheniformis MaTa3 chromosomal DNA (isolated as described by Pitcher et al., 1989, Letters in Applied Microbiology 8: 151-156), 0.4 μM of each primer, 200 μM each of dATP, dCTP, dGTP, and dTTP, 1×PCR Buffer II with 2.5 mM MgCl₂, and 2.5 units of AmpliTaq Gold™ DNA polymerase. The reactions were performed in a RoboCycler 40 thermacycler programmed for 1 cycle at 95° C. for 10 minutes; 25 cycles each at 95° C. for 1 minute, 50° C. for 1 minute, and 72° C. for 1 minute; and 1 cycle at 72° C. for 7 minutes. The PCR product was visualized in a 2% agarose gel using 0.5×TBE buffer. The expected fragment was approximately 230 bp.

| | |
|---|---|
| Primer 961197: | |
| 5′- | (SEQ ID NO: 49) |
| GGCCTTAAGGGCCTGCTGTCCAGACTGTCCGCT- | |
| 3′ | |
| Primer 94-935: | |
| 5′-GGCGTTACAATTCAAAGA-3′ | (SEQ ID NO: 50) |

### Example 3

### Construction of Bacillus licheniformis Strain MDT217 which Harbors the P17 Promoter/amyL Expression Cassette in the Native amyL Locus

The amyL4199 promoter (Pr_{amyL4199}, U.S. Pat. No. 6,100,063), hereafter designated the P6 promoter), was PCR amplified from Bacillus licheniformis SJ1904 (U.S. Pat. No. 5,733,753) chromosomal DNA using the primers shown below, which incorporate a SfiI site and a SacI site, respectively.

Primer 950872:

(SEQ ID NO: 51)

5′-CCAGGCCTTAAGGGCCGCATGCGTCCTTCTTTGTGCT-3′
Primer 991151:

(SEQ ID NO: 52)

5′-GAGCTCCTTTCAATGTGATACATATGA-3′

PCR amplifications were conducted in triplicate in 50 μl reactions composed of 50 ng of Bacillus licheniformis SJ1904 chromosomal DNA (obtained according to Pitcher et al., 1989, supra), 0.4 μM each of primers 950872 and 991151, 200 μM each of dATP, dCTP, dGTP, and dTTP, 1×PCR Buffer II with 2.5 mM MgCl₂, and 2.5 units of AmpliTaq Gold™ DNA polymerase. The reactions were performed in a RoboCycler 40 thermacycler programmed for 1 cycle at 95° C. for 9 minutes; 30 cycles each at 95° C. for 1 minute, 55° C. for 1 minute, and 72° C. for 1 minute; and 1 cycle at 72° C. for 5 minutes. The PCR product was visualized using a 0.8% agarose gel with 0.5×TBE buffer. The expected fragment was approximately 600 bp.

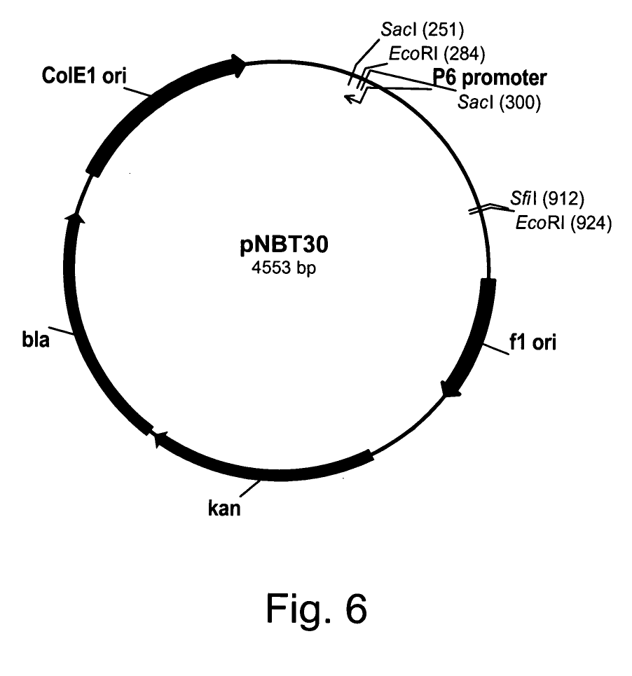

The 600 bp PCR fragment was cloned into pCR2.1 using a TA-TOPO Cloning Kit and transformed into E. coli OneShot™ competent cells according to the manufacturer's instructions (Stratagene, Inc., La Jolla, Calif.). Transformants were selected at 37° C. after 16 hours of growth on 2×YT agar plates supplemented with 100 μg of ampicillin per ml. Plasmid DNA from these transformants was purified using a QIAGEN Bio Robot 9600 according to the manufacturer's instructions and analyzed by digestion with EcoRI and electrophoresis on a 0.8% agarose gel using 0.5×TBE buffer. The plasmid was found to have the correct fragments (approximately 3913 bp and 640 bp). The DNA sequence of the insert DNA was confirmed by DNA sequencing using M13 (−20) forward and M13 reverse primers (Invitrogen, Inc, Carlsbad, Calif.). A plasmid with the correct cloned sequence was designated pNBT30 (FIG. 6).

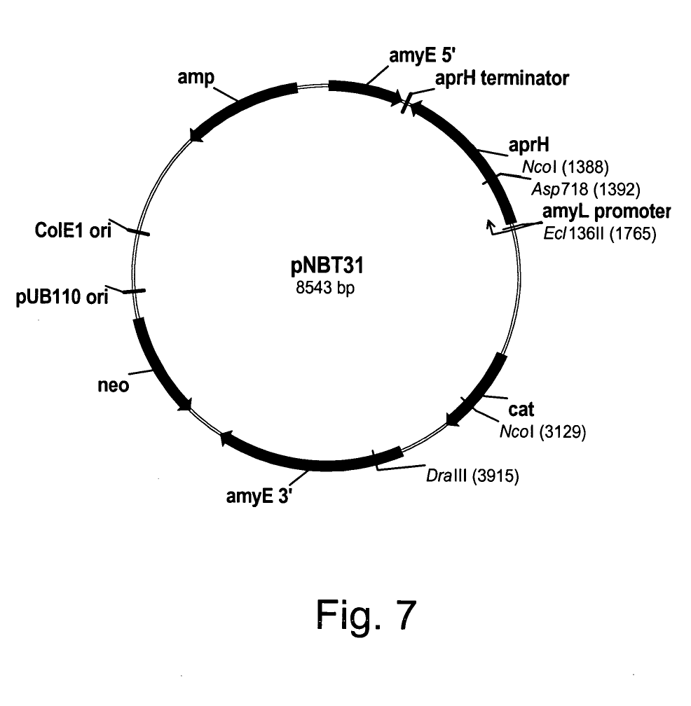

Plasmids pNBT11 (pDG268MCSΔneo-Pr_{cryIIIA}/SAV, U.S. Pat. No. 6,255,076) and pNBT30 were digested with SfiI and SacI. The digestions were resolved on a 0.8% agarose gel using 0.5×TBE buffer and the larger vector fragment (approximately 7931 bp) from pNBT11 and the smaller fragment (approximately 611 bp) from pNBT30 were gel purified using a QIAquick DNA Extraction Kit according to the manufacturer's instructions. The two purified fragments were ligated together with T4 DNA ligase and the ligation mix was transformed into E. coli SURE® competent cells. Transformants were selected on 2×YT agar plates supplemented with 100 μg of ampicillin per ml. Plasmid DNA from these transformants was purified using a QIAGEN Bio Robot 9600 according to the manufacturer's instructions and analyzed by digestion with NcoI and electrophoresis on a 0.8% agarose gel using 0.5×TBE buffer. The plasmid was found to have the correct fragments (approximately 6802 bp and 1741 bp) and was designated pNBT31 (FIG. 7).

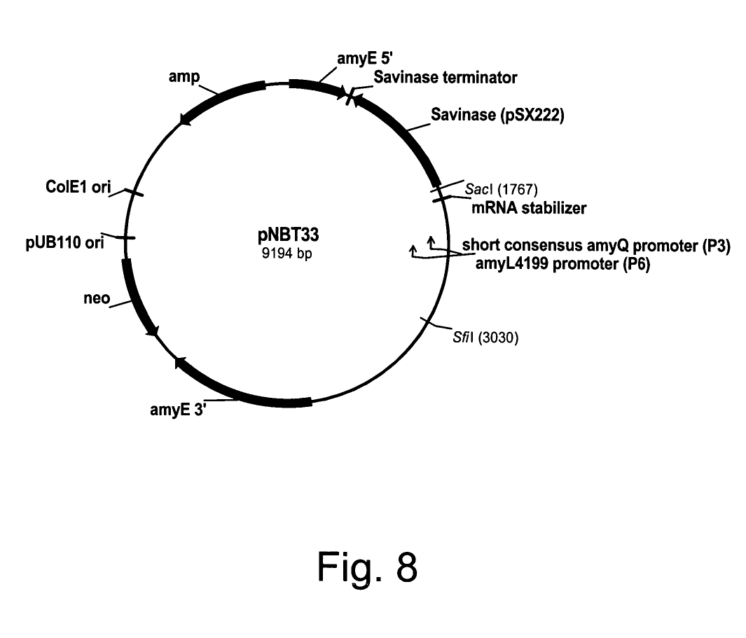

A Pr_{amyL}4199/Pr_{short “consensus” amyQ}/cryIIIAstab composite promoter (P16) was constructed as follows. Plasmid pNBT31 was digested with DraIII and Ecl136II. Plasmid pNBT24 (pDG268MCSΔneo-Pr_{short “consensus” amyQ}/long cryIIIAstab/SAV, U.S. Pat. No. 6,255,076) was digested with SfiI, the ends were blunted using T4 DNA polymerase I, and then digested with DraIII. The digestions were resolved on a 0.8% agarose gel using 0.5×TBE buffer and the larger vector fragment from pNBT31 and the smaller promoter fragment (approximately 1100 bp) from pNBT24 were gel purified using a QIAquick DNA Extraction Kit according to the manufacturer's instructions. The two purified fragments were ligated together with T4 DNA ligase and the ligation mix was transformed into E. coli SURE® competent cells according to the manufacturer's instructions. Transformants were selected on 2×YT agar plates supplemented with 100 μg of ampicillin per ml. Plasmid DNA was purified from several transformants using a QIAGEN Bio Robot 9600 according to the manufacturer's instructions and analyzed by SfiI plus SacI digestion on a 0.8% agarose gel using 0.5×TBE buffer. The correct plasmid was identified by the presence of an approximately 1400 bp SfiI/SacI fragment and was designated pNBT33 (FIG. 8).

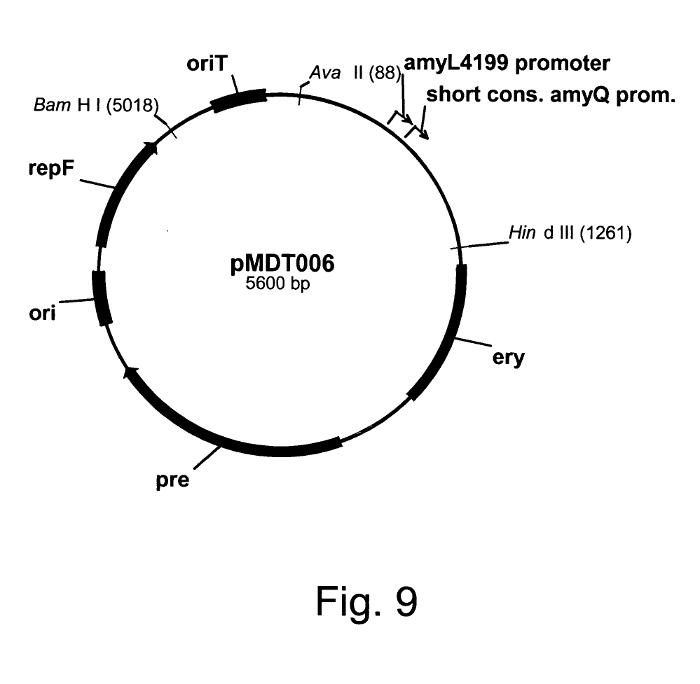

Plasmid pNBT33 was digested with AvaIl and Ecl136II. Plasmid pMRT074 (WO 03/054163) was digested with NotI, the ends were blunted using T4 DNA polymerase, and then digested with AvaIl. The digestions were resolved on a 0.8% agarose gel using 0.5×TBE buffer and the larger vector fragment (approximately 4433 bp) from pMRT074 and the smaller fragment (approximately 1157 bp) from pNBT33 were gel purified using a QIAquick DNA Extraction Kit according to the manufacturer's instructions. The two purified fragments were ligated together with T4 DNA ligase and the ligation mix was transformed into Bacillus subtilis 168Δ4. Transformants were selected on TBAB-agar plates supplemented with 1 μg of erythromycin and 25 μg of lincomycin per ml after incubation at 28° C. for 24-48 hours. Plasmid DNA from several transformants was isolated using QIAGEN tip-20 columns according to the manufacturer's instructions and verified by digestion with BamHI plus HindIII. The resulting plasmid was designated pMDT006 (FIG. 9).

Plasmid pMDT006 was introduced into Bacillus licheniformis MaTa3 via electroporation. An erythromycin-resistant transformant was isolated and the plasmid was integrated and excised from the chromosome as described in Example 1, which resulted in the replacement of the P12 tandem promoter with the P16 promoter yielding Bacillus licheniformis strain MDT216. Bacillus licheniformis MDT216 is essentially a rifampicin-sensitive version of Bacillus licheniformis MDT206 (see Example 6). The desired clone was identified by PCR analysis using primers 94-935 and 94-919 (above).

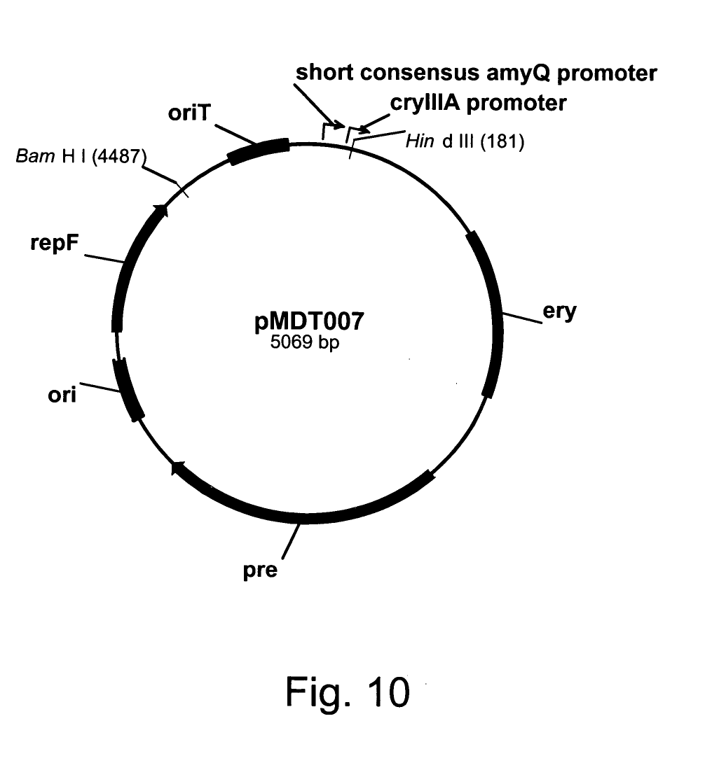

Plasmid pWWi005 (Example 2) was digested with SfiI and EcoRI to remove the DNA sequence located upstream of the amyL gene. The ends were blunted with T4 DNA polymerase and the fragments were resolved on a 0.8% agarose gel using 0.5×TBE buffer. The larger vector fragment (approximately 5069 bp) was gel purified using a QIAquick DNA Extraction Kit according to the manufacturer's instructions. The purified fragment was ligated together with T4 DNA ligase and the ligation mix was transformed into Bacillus subtilis 168Δ4. Transformants were selected on TBAB-agar plates supplemented with 1 μg of erythromycin and 25 μg of lincomycin per ml after incubation at 28° C. for 24-48 hours. Plasmid DNA from several transformants was isolated using QIAGEN tip-20 columns according to the manufacturer's instructions and verified by digestion with BamHI plus HindIII. The resulting plasmid was designated pMDT007 (FIG. 10).

Plasmid pMDT007 was introduced into Bacillus licheniformis MDT216 via electroporation. An erythromycin-resistant transformant was isolated and the plasmid was integrated and excised from the chromosome as described in Example 1, which resulted in the conversion of the P16 promoter to the Pr_{amyL4199}/Pr_{short “consensus” amyQ}/Pr_{cryIIIA}/cryIIIAstab triple tandem promoter (P17) yielding Bacillus licheniformis strain MDT217. The desired clone was identified by PCR analysis using primers 94-935 and 94-919 described above.

### Example 4

### Construction of Bacillus licheniformis MDT220, a C-component protease-deleted derivative of Bacillus licheniformis MDT217

Bacillus licheniformis MDT220, a C-component-negative strain containing the amyL gene (accession no. M13256) under control of the P17 triple tandem promoter, was constructed by deletion of the C-component protease gene of Bacillus licheniformis MDT217 (Example 3) as described below.

A deleted version of the gene encoding Bacillus licheniformis C-component protease (U.S. Pat. No. 5,459,064, accession no. D10060, Kakudo et al., 1992, J. Biol. Chem. 267: 23782) was constructed by PCR using SOE (Horton et al., 1989, supra). The 5′ and 3′ regions of the C-component gene were PCR amplified from Bacillus licheniformis SJ1904 DNA (Example 3) using primer 991173 (which introduced a 5′ EcoRI restriction site) and primer 991174 for the 5° C.-component fragment and primers 991175 and 991176 (which introduced a 3′ HindIII restriction site) for the 3′ C-component fragment, shown below.

Primer 991173:

(SEQ ID NO: 53)

5′-GAATTCGACGGCTTCCCGTGCGCC-3′
Primer 991174:

(SEQ ID NO: 54)

5′-GCAAGCGAGCACGGATTGTAAGTACAAGTTAGATA-3′
Primer 991175:

(SEQ ID NO: 55)

5′-AACTTGTACTTACAATCCGTGCTCGCTTGCCGTAC-3′
Primer 991176:

(SEQ ID NO: 56)

5′-AAGCTTCCATTCAAACCTGGTGAGGAAG-3′

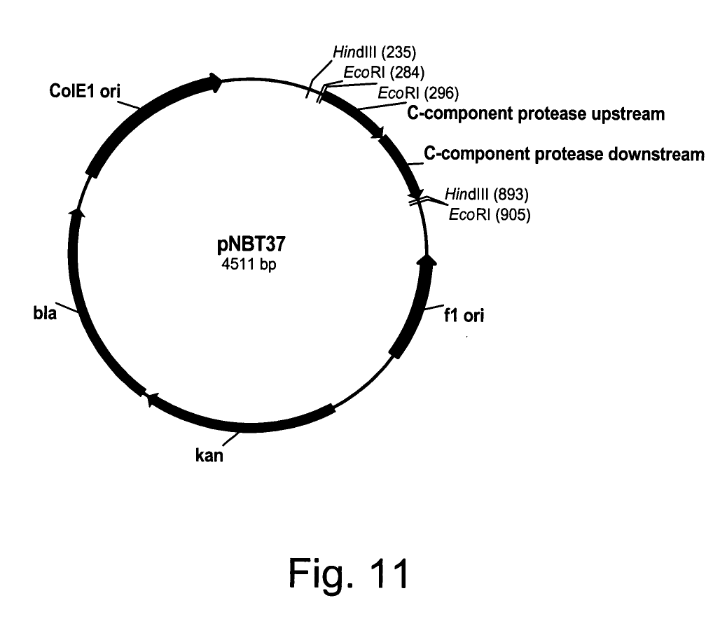

PCR amplifications were carried out in triplicate in 30 μl reactions composed of 50 ng of Bacillus licheniformis SJ1904 chromosomal DNA (obtained according to Pitcher et al., 1989, supra), 0.4 μM each of primer pair 991173 and 991174 for the 5° C.-component fragment or primer pair 991175 and 991176 for the 3° C.-component fragment, 200 μM each of dATP, dCTP, dGTP, and dTTP, 1×PCR Buffer II with 2.5 mM MgCl₂, and 2.5 units of AmpliTaq Gold™ DNA polymerase. The reactions were performed in a RoboCycler 40 thermacycler programmed for 1 cycle at 95° C. for 9 minutes; 3 cycles each at 95° C. for 1 minute, 52° C. for 1 minute, and 72° C. for 1 minute; 27 cycles each at 95° C. for 1 minute, 55° C. for 1 minute, and 72° C. for 1 minute; and 1 cycle at 72° C. for 5 minutes. The PCR products were visualized using a 0.8% agarose gel with 0.5×TBE buffer. The expected fragments were approximately 290 bp in size. The final SOE fragment was generated using primer pair 991173 and 991176 according to Horton et al., 1989, supra, and cloned into pCR2.1 using a TA-TOPO Cloning Kit. Transformants were selected on 2×YT agar plates supplemented with 100 μg of ampicillin per ml after incubation at 37° C. for 16 hours. Plasmid DNA from several transformants was isolated using QIAGEN tip-20 columns according to the manufacturer's instructions and verified by DNA sequencing with M13 (−20) forward and M13 reverse primers. The plasmid harboring the SOE fragment was designated pNBT37 (FIG. 11).

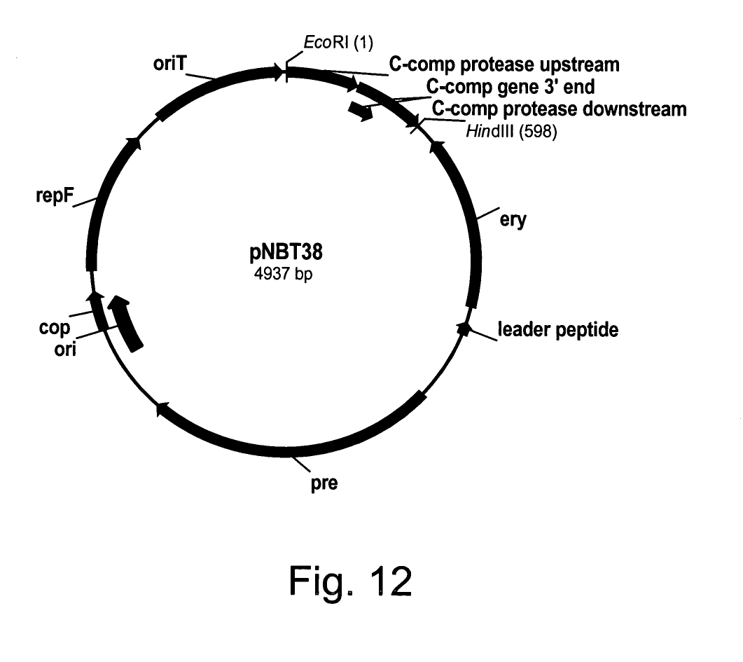

Plasmids pCJ791 (WO 03/054163) and pNBT37 were digested with EcoRI and HindIII. The digestions were resolved on a 0.8% agarose gel using 0.5×TBE buffer and the larger vector fragment (approximately 4340 bp) from pCJ791 and the smaller C-component deletion fragment (approximately 580 bp) from pNBT37 were gel purified using a QIAquick DNA Extraction Kit according to the manufacturer's instructions. The two purified fragments were ligated together with T4 DNA ligase and the ligation mix was transformed into Bacillus subtilis 168Δ4. Transformants were selected on TBAB-agar plates supplemented with 1 μg of erythromycin and 25 μg of lincomycin per ml after incubation at 28° C. for 24-48 hours. Plasmid DNA from several transformants was isolated using QIAGEN tip-20 columns according to the manufacturer's instructions and verified by digestion with EcoRI plus HindIII and agarose gel electrophoresis. A plasmid was identified, which yielded the expected fragment sizes of approximately 4340 bp and approximately 580 bp, and was designated pNBT38 (FIG. 12).

Plasmid pNBT38 was introduced into Bacillus licheniformis MDT217 via electroporation. An erythromycin resistant transformant was isolated and the plasmid was integrated and excised from the chromosome as described in Example 1. Chromosomal DNA was isolated from several transformants according to Pitcher et al., 1989, supra, and was analyzed by PCR using primers 991173 and 991176 as described above to identify the C-component-deleted strains. Several transformants were identified which yielded the expected size PCR fragment of approximately 580 bp which confirmed a partial deletion of the C-component gene. One of the transformants was chosen and designated Bacillus licheniformis MDT220.

### Example 5

### Construction of Plasmid pTH012

The gene encoding the Bacillus sp. JP170 alkaline protease (WO 98/56927 and U.S. Pat. No. 5,891,701, accession no. AR069954) was PCR amplified from Bacillus sp. JP170 chromosomal DNA using the primers below.

| Primer 992843: | |
|---|---|
| 5′- | (SEQ ID NO: 57) |
| CGAGCTCGATGTGTTATAAATTGAGAGGAG-3′ | |
| Primer 961252: | |
| 5′- | (SEQ ID NO: 58) |
| GCGGCCGCGTCATAAACGTTGCAATCGTGCTC- | |
| 3′ | |

PCR amplifications were conducted in triplicate in 50 μl reactions composed of 50 ng of Bacillus sp. JP170 chromosomal DNA (obtained according to Pitcher et al., 1989, supra), 0.4 μM each of primers 992843 and 961252, 200 μM each of dATP, dCTP, dGTP, and dTTP, 1×PCR Buffer II with 2.5 mM MgCl₂, and 2.5 units of AmpliTaq Gold™ DNA polymerase. The reactions were performed in a RoboCycler 40 thermacycler programmed for 1 cycle at 95° C. for 9 minutes; 30 cycles each at 95° C. for 1 minute, 55° C. for 1 minute, and 72° C. for 1 minute; and 1 cycle at 72° C. for 5 minutes. The PCR product was visualized using a 0.8% agarose gel with 0.5×TBE buffer. The expected fragment was approximately 2163 bp.

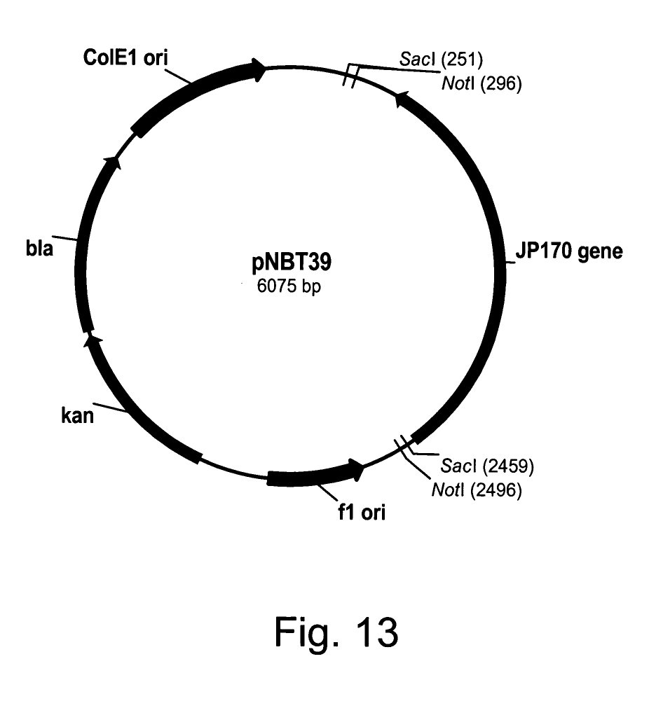

The 2163 bp PCR fragment was cloned into pCR2.1 using a TA-TOPO Cloning Kit and transformed into E. coli OneShot™ competent cells according to the manufacturer's instructions. Transformants were selected at 37° C. after 16 hours of growth on 2×YT agar plates supplemented with 100 μg of ampicillin per ml. Plasmid DNA was purified from several transformants using a QIAGEN Bio Robot 9600 according to the manufacturer's instructions and analyzed by SacI plus NotI digestion on a 0.8% agarose gel using 0.5×TBE buffer. Plasmids with the correct size inserts were identified and the DNA sequence of the inserts was confirmed by DNA sequencing using M13 (−20) forward and M13 reverse primers and the following internal primers. A plasmid with the correct sequence was identified and designated pNBT39 (FIG. 13).

Primer 992843:

(SEQ ID NO: 59)

5′-CGAGCTCGATGTGTTATAAATTGAGAGGAG-3′
Primer 961021:

(SEQ ID NO: 60)

5′-GTCGAATATGATGGGGATG-3′
Primer 960898:

(SEQ ID NO: 61)

5′-GGACAAGGACAGATTGTAGCAGTTGCTGATACTGG-3′
Primer 961048:

(SEQ ID NO: 62)

5′-GCGATTACAGTTGGGGCAACC-3′
Primer 961222:

(SEQ ID NO: 63)

5′-GGTAGCACGACGGCATCACTAAC-3′

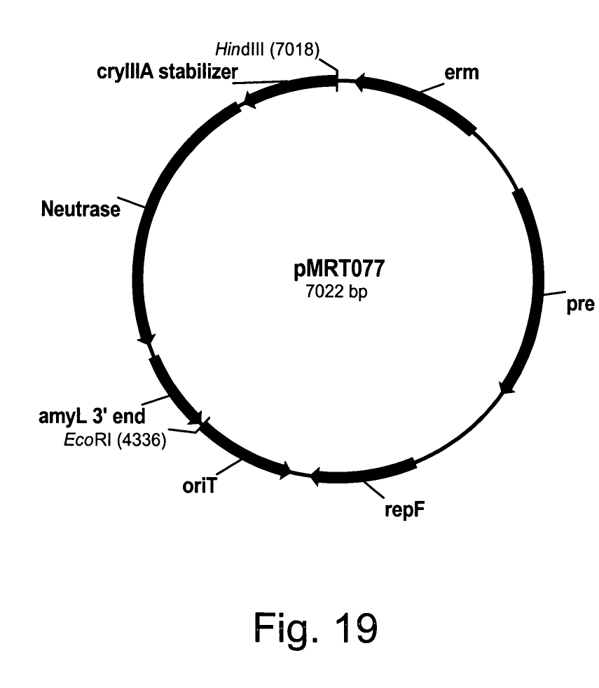

Plasmid pMRT077 (FIG. 19) was constructed as follows. The upstream region and the 3′ region of the amyL gene were fused via SOE using primer pair 733-45-1 and 733-45-7 and primer pair 757-19-1 and 733-45-6, respectively, shown below.

Primer 733-45-1:

| | |
|---|---|
| 5′-GTCCTTCTTGGTACCTGGAAGCAGAGC-3′ | (SEQ ID NO: 64) |
| Primer 733-45-7: | |
| 5′-CATGCTGGGCCCTTAAGGCCAGTACCATTTTCCC-3′ | (SEQ ID NO: 65) |
| Primer 757-19-1: | |
| 5′-CAGTAGGCCTTAAGGGCCCAGCATGATTGAGCTCACCACCATGGGATCCGCGG | (SEQ ID NO: 66) |
| CCGCACAAGGGAAGGC-3′ | |
| Primer 733-45-6: | |
| 5′-CAATTCATCCTCTAGAGTCTCAGG-3′ | (SEQ ID NO: 67) |

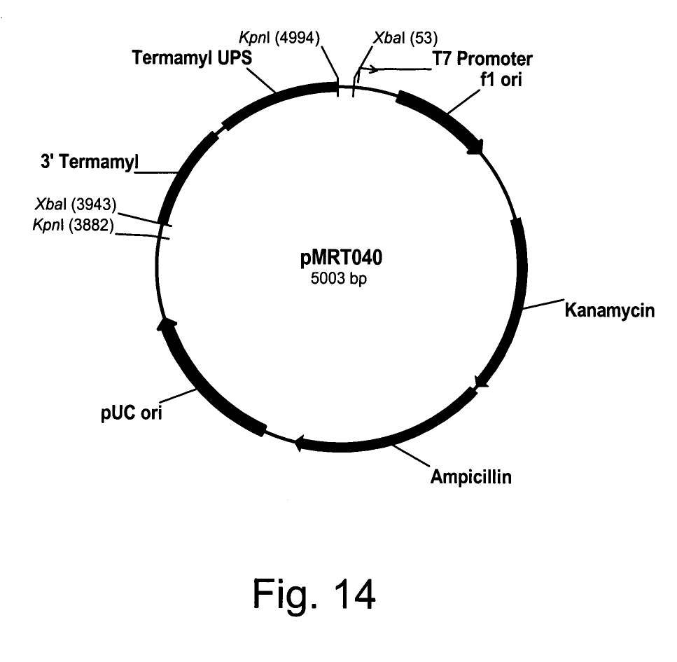

PCR amplification was conducted in 50 μl reactions composed of 1 ng of pDN1981 DNA (U.S. Pat. No. 5,698,415), 0.4 μM of each primer, 200 μM each of dATP, dCTP, dGTP, and dTTP, 1×PCR Buffer II with 2.5 mM MgCl₂, and 2.5 units of AmpliTaq Gold™ DNA polymerase. The reactions were performed in a RoboCycler 40 thermacycler programmed for 1 cycle at 95° C. for 10 minutes; 25 cycles each at 95° C. for 1 minute, 50° C. for 1 minute, and 72° C. for 1 minute; and 1 cycle at 72° C. for 7 minutes. The PCR products were visualized by 0.8% agarose gel electrophoresis using 0.5×TBE buffer. The expected fragments were approximately 600 and 500 bp, respectively. The final fragment was amplified using primers 733-45-1 and 73345-6. The final fragment was cloned into pCR2.1 vector using a TA-TOPO Cloning Kit. Transformants were selected on 2×YT agar plates supplemented with 100 μg of ampicillin per ml and incubated at 37° C. for 16 hours. Transformants carrying the correct plasmid were verified by DNA sequencing using M13 forward and reverse primers. The plasmid was designated pMRT040 (FIG. 14).

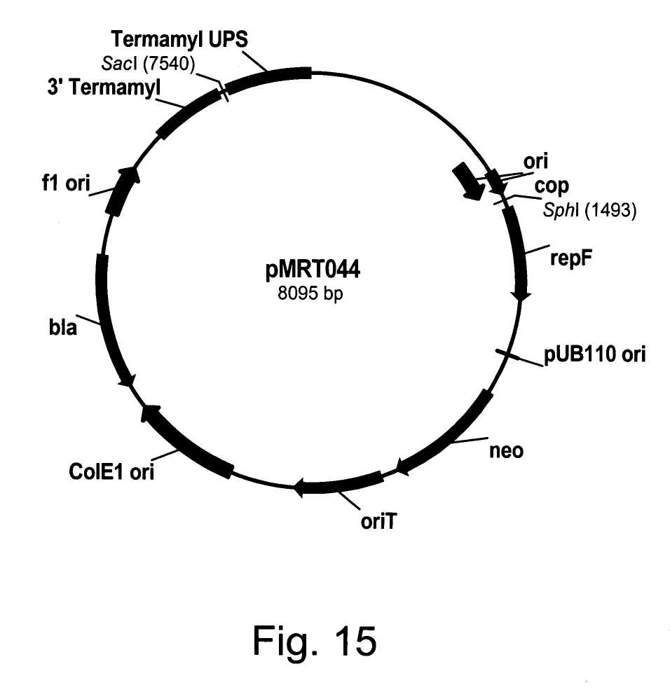

Plasmid pMRT040 was digested with KpnI/XbaI and filled-in with Klenow fragment, and a fragment of approximately 1000 bp was isolated from a 0.8% agarose-0.5×TBE gel using a QIAquick DNA Purification Kit according to the manufacturer's instructions. This fragment was cloned into plasmid pShV3 (WO 03/054163) digested with EcoRV, and transformed into E. coli XL1 Blue cells according to the manufacturer's instructions (Stratagene, Inc., La Jolla, Calif.). Transformants were selected on 2×YT agar plates supplemented with 100 μg of ampicillin per ml and incubated at 37° C. for 16 hours. Plasmid DNA from several transformants was isolated using QIAGEN tip-20 columns according to the manufacturer's instructions and verified on a 0.8% agarose gel using 0.5×TBE buffer by restriction analysis with SacI/SphI. The resulting plasmid was designated pMRT044 (FIG. 15).

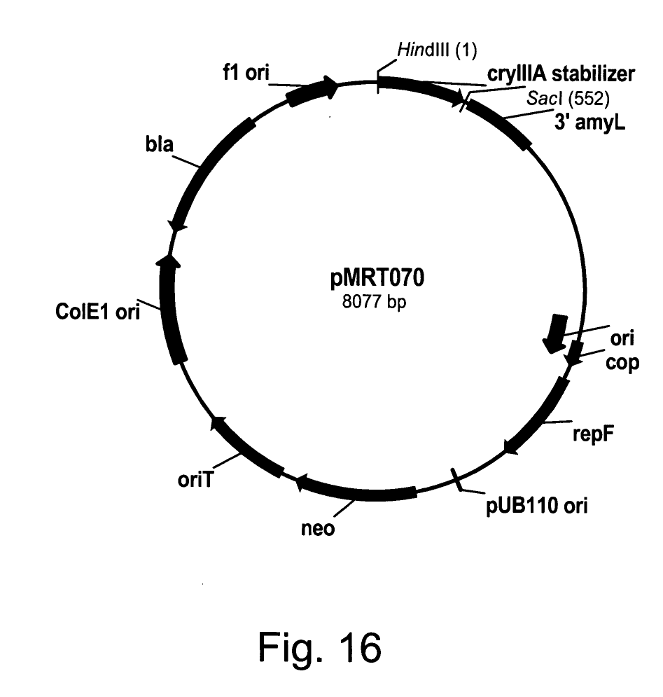

Plasmid pMRT044 and pNBT3 (pDG268MCSΔneo-Pr_{cryIIIA}/cryIIIAstab/SAV, U.S. Pat. No. 6,255,076) were digested with SacI plus HindIII. The digestions were resolved on a 0.8% agarose gel using 0.5×TBE buffer and the larger vector fragment (approximately 7500 bp) from pMRT044 and the smaller cryIIIA stabilizer fragment (approximately 540 bp) from pNBT3 were gel purified using a QIAquick DNA Extraction Kit according to the manufacturer's instructions. The two purified fragments were ligated together with T4 DNA polymerase and the ligation mix was used to transform E. coli XL1 Blue cells according to the manufacturer's instructions. Transformants were selected on 2×YT agar plates supplemented with 100 μg of ampicillin per ml and incubated at 37° C. for 16 hours. Plasmid DNA from several transformants was isolated using QIAGEN tip-20 columns according to the manufacturer's instructions and verified by restriction analysis with SacI/HindIII on a 0.8% agarose gel using 0.5×TBE buffer. The resulting plasmid was designated pMRT070 (FIG. 16).

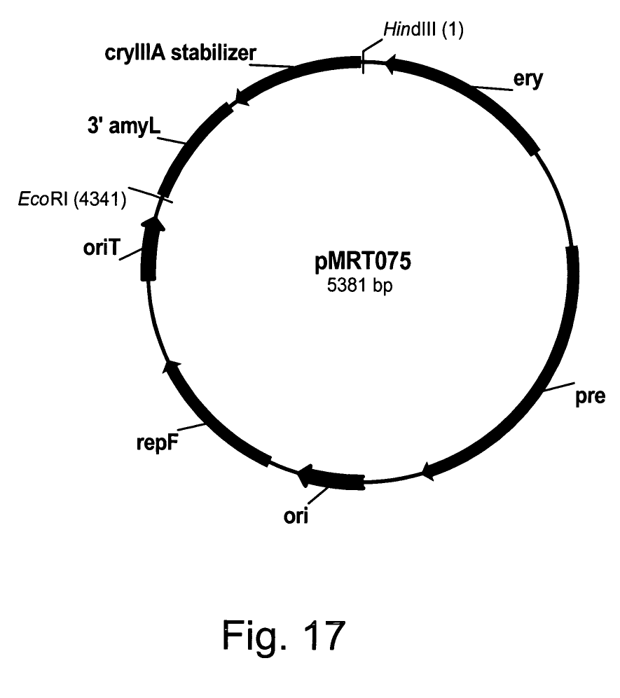

Plasmids pMRT074 (WO 03/054163) and pMRT070 were digested with EcoRI/HindIII. A fragment of approximately 3500 bp from pMRT074 and a fragment of approximately 1100 bp from pMRT070 were isolated from a 0.8% agarose gel using 0.5×TBE buffer and a QIAquick DNA Purification Kit according to the manufacturer's instructions, ligated, and transformed into Bacillus subtilis 168Δ4 competent cells. Transformants were selected on TBAB-agar plates supplemented with 1 μg of erythromycin and 25 μg of lincomycin per ml and incubated at 30° C. for 24-48 hours. Plasmid DNA from several transformants was isolated using QIAGEN tip-20 columns according to the manufacturer's instructions and verified by restriction analysis with EcoRI/HindIII on a 0.8% agarose gel using 0.5×TBE buffer. The resulting plasmid was designated pMRT075 (FIG. 17).

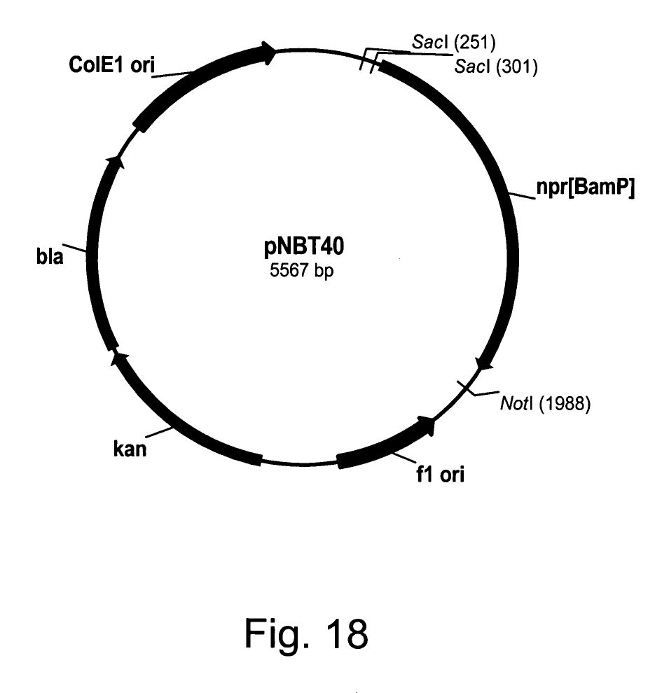

Plasmids pMRT075 and pNBT40 (FIG. 18) were digested with SacI plus NotI. Plasmid pNBT40 is essentially the pCR2.1-TOPO vector containing a gene (npr[BamP]) encoding a neutral protease from Bacillus amyliquefaciens (Vasantha et al., 1984, J. Bacteriol. 159: 811, accession no. K02497). A DNA fragment of approximately 5500 bp from pMRT075 and a fragment of approximately 1600 bp from pNBT40 were isolated from a 0.8% agarose gel using 0.5×TBE buffer and a QIAquick DNA Purification Kit according to the manufacturer's instructions, ligated, and transformed into Bacillus subtilis 168Δ4 competent cells. Transformants were selected on TBAB-agar plates supplemented with 1% skim milk, and 1 μg of erythromycin and 25 μg of lincomycin per ml and incubated at 30° C. for 24-48 hours. Transformants producing clearing zones on TBAB-agar skim milk plates were obtained and the resulting plasmid was designated pMRT077 (FIG. 19).

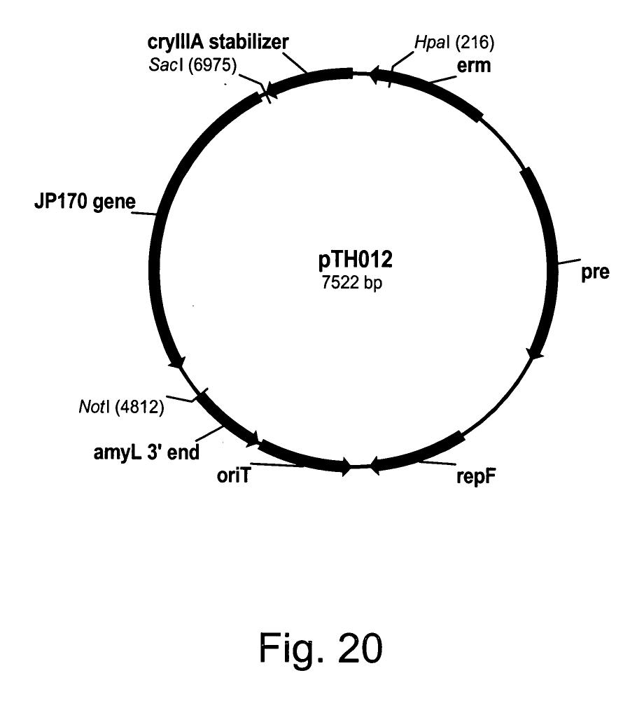

Plasmid pNBT39 and pMRT077 were digested with SacI plus NotI. The digestions were resolved on a 0.8% agarose gel using 0.5×TBE buffer and the larger vector fragment (approximately 5400 bp) from pMRT077 and the smaller JP170 protease fragment (approximately 2163 bp) from pNBT39 were gel purified using a QIAquick DNA Extraction Kit according to the manufacturer's instructions. The two purified fragments were ligated together with T4 DNA ligase and the ligation mix was transformed into Bacillus subtilis 168Δ4 competent cells. Plasmid DNA was purified from several transformants using QIAGEN tip-20 columns according to the manufacturer's instructions and analyzed by SacI plus NotI digestion on a 0.8% agarose gel using 0.5×TBE buffer. The correct plasmid was identified by the presence of an approximately 2163 bp SacI/NotI JP170 fragment and was designated pTH012 (FIG. 20).

### Example 6

### Construction of a Bacillus licheniformis Strain TH15 Containing One-Copy of the P17 Promoter/se hasA/tuaD/gtaB Expression Cassette in the amyL Locus

Plasmid pMB748 harbors the mature portion of the mannanase gene from Bacillus sp. 1633 (WO 99/64619) fused to the amyL signal sequence from the Bacillus licheniformis amyL gene. A truncated version of this gene was constructed by deleting the 3′ portion of the mannanase gene using primer pair 80501D1B11 and 172965 shown below and plasmid pMB748 as template DNA. PCR amplifications were conducted in 50 μl reactions composed of 50 ng of pMB748 DNA, 0.4 μM each of primers 80501D1B11 and 172965, 200 μM each of dATP, dCTP, dGTP, and dTTP, 1×PCR Buffer II with 2.5 mM MgCl₂, and 2.5 units of AmpliTaq Gold™ DNA polymerase. The reactions were performed in a RoboCycler 40 thermacycler programmed for 1 cycle at 95° C. for 9 minutes; 30 cycles each at 95° C. for 1 minute, 55° C. for 1 minute, and 72° C. for 1 minute; and 1 cycle at 72° C. for 5 minutes. A PCR product of approximately 900 bp was visualized using a 0.8% agarose gel with 0.5×TBE buffer.

Primer 80501D1B11:

(SEQ ID NO: 68)

5′-CATTCTGCAGCCGCGGCAAATTCCGGATTTTATGTAAGCGG-3′
Primer 172965:

(SEQ ID NO: 69)

5′-
CATCATATGCGGCCGCTTATCATTGAAAAACGGTGCTTAATCTCGAAG-
3′

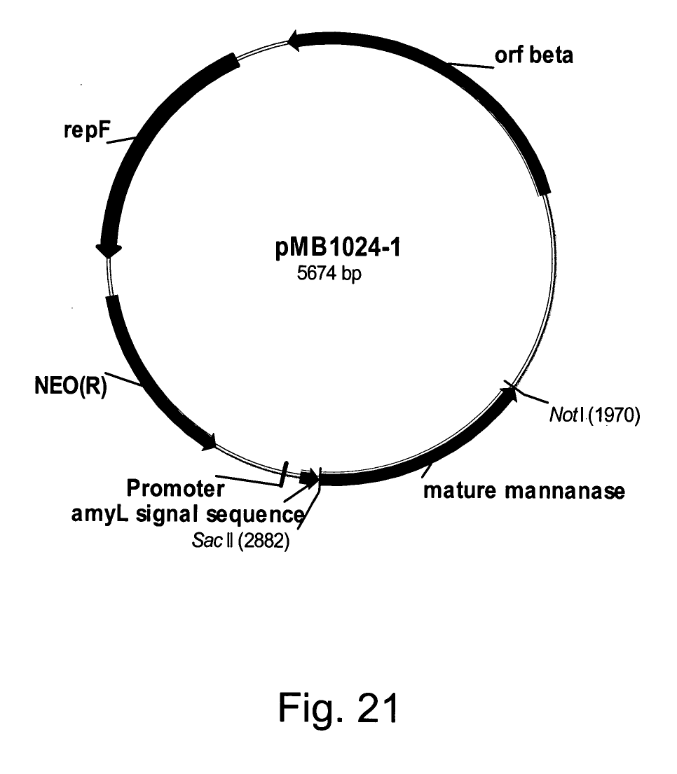

The PCR fragment was cloned into plasmid pMOL944 (WO 99/64619) which was digested with SacI and NotI. The plasmid vector fragment and the amyL/mannanase PCR fragment were isolated from a 0.8% agarose-0.5×TBE gel using a QIAquick DNA Purification Kit according to the manufacturer's instructions, ligated, and transformed into Bacillus subtilis strain PL2306 (U.S. Pat. No. 6,677,147) selecting for kanamycin resistance on TBAB plates supplemented with 10 μg of kanamycin per ml. Plasmids from several transformants were purified using a QIAprep Spin Miniprep Kit (QIAGEN Inc., Valencia, Calif.) according to the manufacturer's instructions. The plasmids were analyzed by restriction enzyme digestion. A plasmid was identified which contained the correct insert and was designated pMB1024-1 (FIG. 21).

Bacillus subtilis MDT206 was constructed as follows. Plasmid pMDT006 (Example 3) was introduced into Bacillus licheniformis MaTa4 via electroporation. An erythromycin resistant transformant was isolated and the plasmid was integrated and excised from the chromosome as described in Example 1, which resulted in the replacement of the P11 promoter with the P16 promoter yielding Bacillus licheniformis strain MDT206. The desired clone was identified by PCR analysis using primer 94-935 (above) and the following primer:

| | |
|---|---|
| Primer 94-919: | |
| 5′-GGAAGTACAAAAATAAGC-3′ | (SEQ ID NO: 70) |

The cryIIIA mRNA processing stabilizer sequence and amyL signal sequence were PCR amplified and cloned from Bacillus licheniformis strain MDT206 as follows. PCR amplifications were conducted in 50 μl reactions composed of 50 ng of Bacillus licheniformis MDT206 chromosomal DNA (obtained according to Pitcher et al., 1989, supra), 0.4 μM each of primers 226370 and 219916, 200 μM each of dATP, dCTP, dGTP, and dTTP, 1×PCR Buffer II with 2.5 mM MgCl₂, and 2.5 units of AmpliTaq Gold™ DNA polymerase. The reactions were performed in a RoboCycler 40 thermacycler programmed for 1 cycle at 95° C. for 9 minutes; 30 cycles each at 95° C. for 1 minute, 55° C. for 1 minute, and 72° C. for 1 minute; and 1 cycle at 72° C. for 5 minutes. A PCR product of approximately 650 bp was visualized using a 0.8% agarose gel with 0.5×TBE buffer.

Primer 226370:

(SEQ ID NO: 71)

5′-CATCCCCCCGGGAGCTTAATTAAAGATAATATCTTTGAATTG-3′
Primer 219916:

(SEQ ID NO: 72)

5′-TGCCGCGGCTGCAGAATGAGGCAG-3′

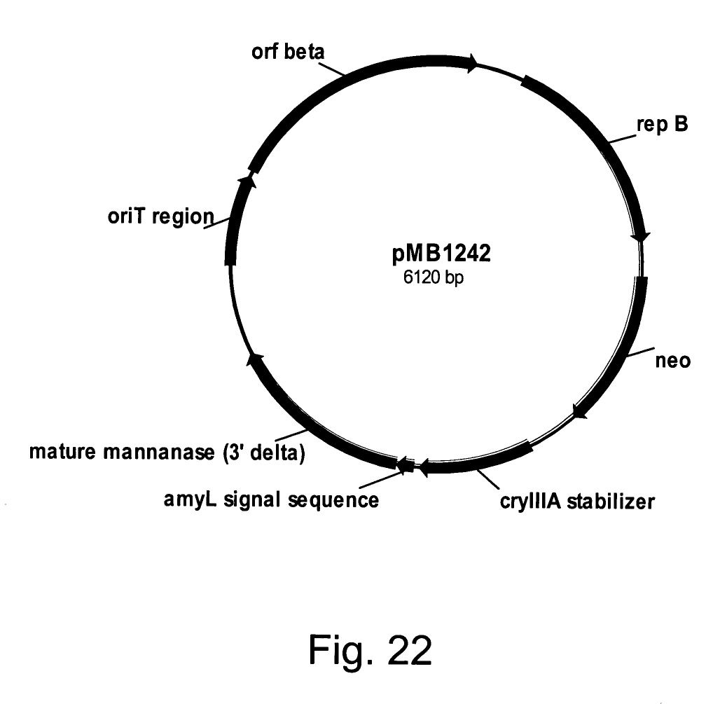

Plasmid pMB1024-1 and the cryIIIA/amyL signal sequence PCR fragment were digested with XmaI and PstI. The larger vector fragment (approximately 5500 bp) from pMB1024-1 and the cryIIIA/amyL signal sequence PCR fragment (approximately 650 bp) were isolated from a 0.8% agarose-0.5×TBE gel using a QIAquick DNA Purification Kit according to the manufacturer's instructions, ligated, and transformed into Bacillus subtilis strain MOL2023 selecting for kanamycin resistance on TBAB plates supplemented with 10 μg of kanamycin per ml. This strain is essentially Bacillus subtilis A164Δ10 with the erythromycin resistance gene from plasmid pE194 (Horinouchi and Weisblum, 1982, J. Bacteriol. 150: 804-814) inserted into the ydhT gene (encodes a mannan endo-1,4-beta-mannosidase). Bacillus subtilis A164Δ10 is derived from Bacillus subtilis A164Δ5 (U.S. Pat. No. 5,891,701) and has deletions in the following genes: spoIIAC, aprE, nprE, amyE, srfAC, wprA, bpr, vpr, mpr, and epr. Plasmid DNA from several transformants was purified using a QIAprep Spin Miniprep Kit according to the manufacturer's instructions and analyzed by restriction enzyme digestion. A plasmid containing the correct insert was identified and designated pMB1242 (FIG. 22).

The RBS and mannanase gene were PCR amplified from pMB1242 using primer 993634 shown below and primer 733-68-1 (Example 1).

| | |
|---|---|
| Primer 993634: | |
| 5′-GTTAACTTGAAAAACGGTGCTTAATC-3′ | (SEQ ID NO: 73) |

PCR amplifications were conducted in triplicate in 50 μl reactions composed of 50 ng of pMB1242, 0.4 μM each of primers 993634 and 733-68-1, 200 μM each of dATP, dCTP, dGTP, and dTTP, 1×PCR Buffer II with 2.5 mM MgCl₂, and 2.5 units of AmpliTaq Gold™ DNA polymerase. The reactions were performed in a RoboCycler 40 thermacycler programmed for 1 cycle at 95° C. for 9 minutes; 30 cycles each at 95° C. for 1 minute, 55° C. for 1 minute, and 72° C. for 1 minute; and 1 cycle at 72° C. for 5 minutes. The PCR product was visualized using a 0.8% agarose gel using 0.5×TBE buffer. The expected fragment was approximately 1052 bp.

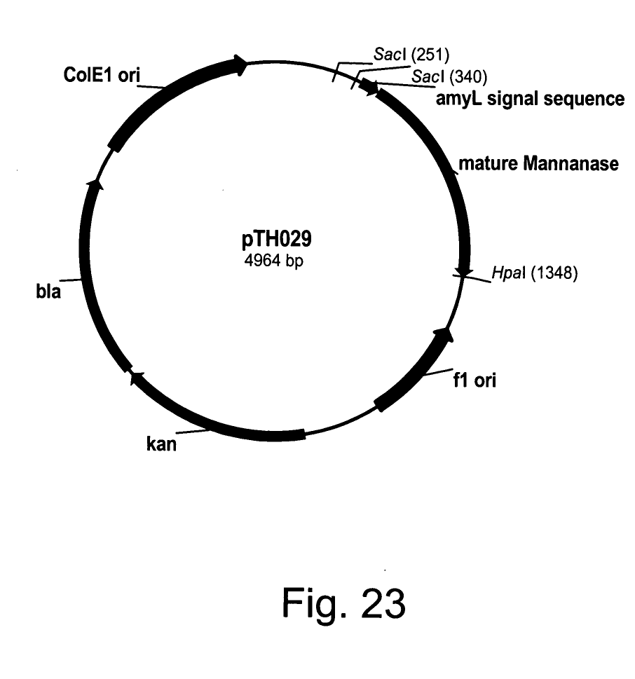

The 1052 bp PCR fragment was cloned into pCR2.1 using a TA-TOPO Cloning Kit and transformed into E. coli OneShot™ competent cells according to the manufacturer's instructions. Transformants were selected at 37° C. after 16 hours of growth on 2×YT agar plates supplemented with 100 μg of ampicillin per ml. Plasmid DNA from these transformants was purified using a QIAGEN Bio Robot 9600 according to the manufacturer's instructions and digested with SacI plus NotI. Plasmids were identified and the DNA sequence of the inserts was confirmed by DNA sequencing using M13 (−20) forward and M13 reverse primers. A plasmid with the correct sequence was identified and designated pTH029 (FIG. 23).

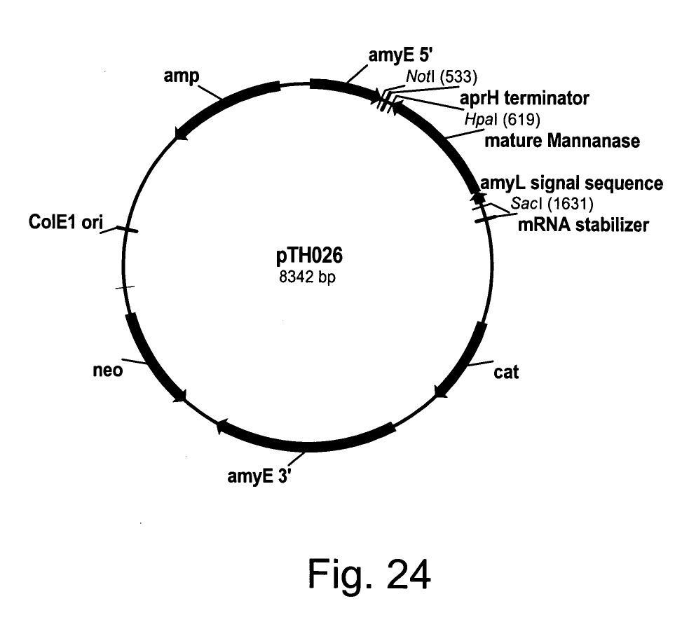

The mannanase gene was next cloned into pNBT18 (pDG268MCSΔneo-long cryIIIAstab/SAV, U.S. Pat. No. 6,255,076) as a SacI/HpaI fragment, replacing the aprH coding region as follows. Plasmid pTH029 was digested with SacI plus HpaI. Plasmid pNBT18 was digested with SacI plus HpaI. The digestions were resolved on a 0.8% agarose gel using 0.5×TBE buffer and the larger vector fragment (approximately 7330 bp) from pNBT18 and the smaller mannanase gene fragment (approximately 1010 bp) from pTH029 were gel purified using a QIAquick DNA Extraction Kit according to the manufacturer's instructions. The two purified fragments were ligated together with T4 DNA ligase and the ligation mix was transformed into E. coli OneShot™ competent cells according to the manufacturer's instructions. Transformants were selected at 37° C. after 16 hours of growth on 2×YT agar plates supplemented with 100 μg of ampicillin per ml. Plasmid DNA from these transformants was purified using a QIAGEN robot according to the manufacturer's instructions. The correct plasmid was identified by the presence of an approximately 1095 bp SacI/NotI mannanase gene fragment and was designated pTH026 (FIG. 24).

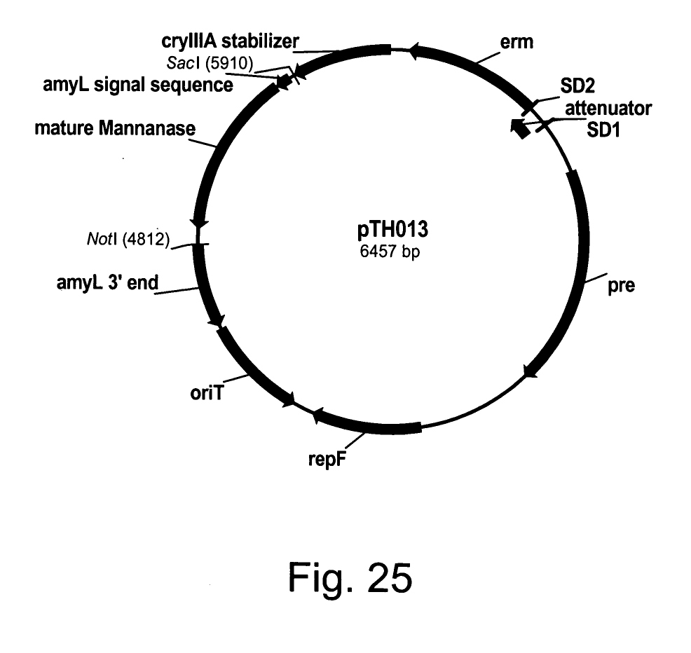

The mannanase gene and aprH gene terminator from pTH026 were then inserted into pTH012 as a SacI/NotI fragment, replacing the JP170 protease gene as follows. Plasmids pTH012 and pTH026 were digested with SacI and NotI. The digestions were resolved on a 0.8% agarose gel using 0.5×TBE buffer and the larger vector fragment (approximately 5359 bp) from pTH012 and the smaller mannanase gene fragment (approximately 1095 bp) from pTH026 were gel purified using a QIAquick DNA Extraction Kit according to the manufacturer's instructions. The two purified fragments were ligated together with T4 DNA ligase and the ligation mix was transformed into Bacillus subtilis 168Δ4. Plasmid DNA was purified from several transformants using QIAGEN tip-20 columns according to the manufacturer's instructions and analyzed by SacI plus NotI digestion on a 0.8% agarose gel using 0.5×TBE buffer. The correct plasmid was identified by the presence of an approximately 1095 bp SacI/NotI mannanase gene fragment (this fragment, bearing the amyL RBS, the amyL signal-mannanase coding sequence fusion, and the aprH terminator, will hereinafter be referred to as “the mannanase gene”). The resulting plasmid was designated pTH013 (FIG. 25).

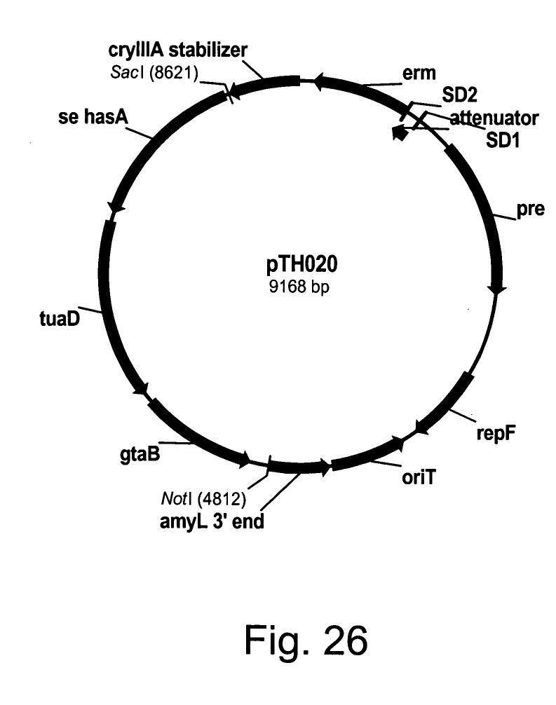

Plasmid pTH020 was constructed to introduce an artificial hyaluronic acid (HA) operon comprising a Streptococcus equisimilis hyaluronan synthase gene (hasA) into the chromosome of Bacillus licheniformis MDT220 under the control of the P17 triple promoter. The cryIIIAstab/hasA/tuaD/gtaB artificial operon from pHA3 (WO 03/054163) was inserted into plasmid pTH013 as a SacI/NotI fragment, replacing the mannanase gene as follows. Plasmids pTH013 and pHA3 were digested with SacI and NotI. The digestions were resolved on a 0.8% agarose gel using 0.5×TBE buffer and the larger vector fragment (approximately 5400 bp) from pTH013 and the smaller cryIIIAstab/hasA/tuaD/gtaB artificial operon fragment (approximately 3800 bp) from pHA3 were gel purified using a QIAquick DNA Extraction Kit according to the manufacturer's instructions. The two purified fragments were ligated together with T4 DNA ligase and the ligation mix was transformed into Bacillus subtilis 168Δ4. Plasmid DNA was purified from several transformants using QIAGEN tip-20 columns according to the manufacturer's instructions and analyzed by SacI plus NotI digestion on a 0.8% agarose gel using 0.5×TBE buffer. The correct plasmid was identified by restriction enzyme digestion and/or PCR analysis. The resulting plasmid was designated pTH020 (FIG. 26).

Plasmid pTH020 was introduced into Bacillus licheniformis MDT220 via electroporation. An erythromycin resistant transformant was isolated and the plasmid was integrated and excised from the chromosome as described in Example 1. Chromosomal DNA was isolated from several transformants (Pitcher et al., 1989, supra) and was analyzed by PCR. A PCR check was performed using primers 992463 and 992468 (hasA gene), 992464 and 992472 (tuaD gene), and 992471 and 992477 (gtaB gene). After the HA operon was integrated into the chromosome, the final strain was identified as being protease-negative and erythromycin-sensitive and the same primers were used again to verify that the operon was present. One of these strains was chosen and designated Bacillus licheniformis TH15.

### Example 7

### Fermentation of Bacillus licheniformis TH15

The production of hyaluronic acid (HA) by Bacillus licheniformis TH15 was evaluated in two-liter fermentors (Applikon, Inc., Holland) with pH controlled at 7.0±0.2 in a medium composed per liter of 6.5 g of KH₂PO₄, 4.5 g of Na₂HPO₄, 3.0 g of (NH₄)₂SO₄, 2.0 g of sodium citrate, 3.0 g of MgSO₄.7H₂O, 15 g of sucrose, 0.5 g of CaCl₂.2H₂O, 6.0 ml of trace metals solution (composition below), and 3.0 ml of defoaming agents. The feed consisted of 20% (w/w) sucrose. The trace metals solution was composed per liter of 100 g of citric acid, 20 g of FeSO₄.7H₂O, 5 g of MnSO₄.H₂O, 2 g of CuSO₄.5H₂O, and 2 g of ZnCl₂. Temperature was controlled at 37° C. during the 48 hour fermentations. Maximum airflow and agitation were 1.5 vvm and 1300 rpm, respectively.

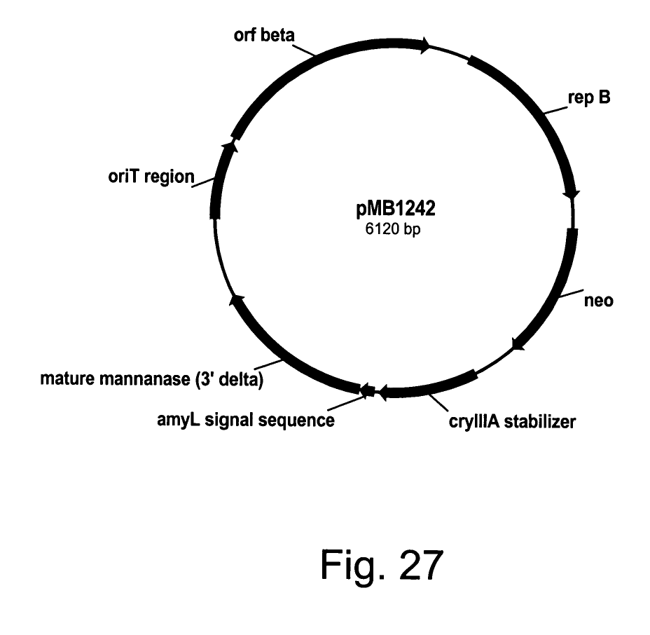

The results shown in FIG. 27 demonstrated the capability of the P17 triple promoter in driving the expression of the hasA/tuaD/gtaB operon to produce HA in Bacillus licheniformis TH15.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

1. A method for producing a hyaluronic acid, comprising:
(a) cultivating a Bacillus cell in a medium conducive for the production of the hyaluronic acid, wherein the Bacillus cell comprises a nucleic acid construct comprising a triple promoter comprising a variant amyL promoter having a mutation corresponding to position 590 of SEQ ID NO: 1, a consensus promoter having the sequence TTGACA for the “−35” region and TATAAT for the “−10” region, and a cryIIIA promoter, in which each promoter sequence of the triple promoter is operably linked to one or more coding sequences involved in the biosynthesis of the hyaluronic acid; and
(b) isolating the hyaluronic acid from the cultivation medium.

2. The method of claim 1, wherein the variant amyL promoter is SEQ ID NO: 1.

3. (canceled)

4. (canceled)

5. (canceled)

6. The method of claim 1, wherein the consensus promoter is obtained from the Bacillus amyloliquefaciens alpha-amylase gene (amyQ).

7. The method of claim 6, wherein the consensus amyQ promoter has the nucleotide sequence of SEQ ID NO: 42 or SEQ ID NO: 43.

8. (canceled)

9. (canceled)

10. The method of claim 1, wherein the nucleic acid construct further comprises an mRNA processing/stabilizing sequence located downstream of the triple promoter and upstream of the one or more coding sequences involved in the biosynthesis of the hyaluronic acid.

11. (canceled)

12. (canceled)

13. (canceled)

14. (canceled)

15. (canceled)

16. (canceled)

17. The method of claim 1, wherein the one or more coding sequences involved in the biosynthesis of the hyaluronic acid are selected from the group consisting of a hyaluronan synthase, UDP-glucose 6-dehydrogenase, UDP-glucose pyrophosphorylase, UDP-N-acetylglucosamine pyrophosphorylase, glucose-6-phosphate isomerase, hexokinase, phosphoglucomutase, amidotransferase, mutase, and acetyl transferase gene.

18. (canceled)

19. (canceled)

20. (canceled)

21. (canceled)

22. (canceled)

23. A Bacillus cell comprising a nucleic acid construct which comprises (a) a triple promoter comprising a variant amyL promoter having a mutation corresponding to position 590 of SEQ ID NO: 1, a consensus promoter having the sequence TTGACA for the “−35” region and TATAAT for the “−10” region, and a cryIIIA promoter, in which each promoter sequence of the triple promoter is operably linked to one or more coding sequences involved in the biosynthesis of a hyaluronic acid, and optionally (b) an mRNA processing/stabilizing sequence located downstream of the triple promoter and upstream of the one or more coding sequences involved in the biosynthesis of the hyaluronic acid.

24. The Bacillus cell of claim 23, wherein the variant amyL promoter is SEQ ID NO: 1.

25. (canceled)

26. (canceled)

27. (canceled)

28. The Bacillus cell of claim 23, wherein the consensus promoter is obtained from the Bacillus amyloliquefaciens alpha-amylase gene (amyQ).

29. The Bacillus cell of claim 28, wherein the consensus amyQ promoter has the nucleotide sequence of SEQ ID NO: 42 or SEQ ID NO: 43.

30. (canceled)

31. (canceled)

32. The Bacillus cell of claim 23, wherein the nucleic acid construct further comprises an mRNA processing/stabilizing sequence located downstream of the triple promoter and upstream of the one or more coding sequences involved in the biosynthesis of the hyaluronic acid.

33. (canceled)

34. (canceled)

35. (canceled)

36. (canceled)

37. (canceled)

38. (canceled)

39. The Bacillus cell of claim 23, wherein the one or more coding sequences involved in the biosynthesis of the hyaluronic acid are selected from the group consisting of a hyaluronan synthase, UDP-glucose 6-dehydrogenase, UDP-glucose pyrophosphorylase, UDP-N-acetylglucosamine pyrophosphorylase, glucose-6-phosphate isomerase, hexokinase, phosphoglucomutase, amidotransferase, mutase, and acetyl transferase gene.

40. (canceled)

41. (canceled)

42. (canceled)

43. (canceled)

44. (canceled)

45. A method for producing a selectable marker-free mutant of a Bacillus cell, comprising deleting a selectable marker gene of the Bacillus cell, wherein the Bacillus cell comprises a nucleic acid construct comprising a triple promoter comprising a variant amyl promoter having a mutation corresponding to position 590 of SEQ ID NO: 1, a consensus promoter having the sequence TTGACA for the “−35” region and TATAAT for the “−10” region, and a cryIIIA promoter, in which each promoter sequence of the triple promoter is operably linked to one or more coding sequences involved in the biosynthesis of a hyaluronic acid.

46. The method of claim 45, wherein the variant amyL promoter is SEQ ID NO: 1.

47. (canceled)

48. (canceled)

49. (canceled)

50. The method of claim 45, wherein the consensus promoter is obtained from the Bacillus amyloliquefaciens alpha-amylase gene (amyQ).

51. The method of claim 50, wherein the consensus amyQ promoter has the nucleotide sequence of SEQ ID NO: 42 or SEQ ID NO: 43.

52. (canceled)

53. (canceled)

54. The method of claim 45, wherein the nucleic acid construct further comprises an mRNA processing/stabilizing sequence located downstream of the triple promoter and upstream of the one or more coding sequences involved in the biosynthesis of the hyaluronic acid.

55. (canceled)

56. (canceled)

57. (canceled)

58. (canceled)

59. (canceled)

60. The method of claim 45, wherein the Bacillus cell contains no foreign selectable marker gene.

61. The method of claim 45, wherein the one or more coding sequences involved in the biosynthesis of the hyaluronic acid are selected from the group consisting of a hyaluronan synthase, UDP-glucose 6-dehydrogenase, UDP-glucose pyrophosphorylase, UDP-N-acetylglucosamine pyrophosphorylase, glucose-6-phosphate isomerase, hexokinase, phosphoglucomutase, amidotransferase, mutase, and acetyl transferase gene.

62. (canceled)

63. (canceled)

64. (canceled)

65. (canceled)

66. (canceled)

67. A selectable marker-free mutant of a Bacillus cell obtained by the method of claim 45.

68. (canceled)

69. (canceled)

70. (canceled)

71. (canceled)

72. (canceled)

73. (canceled)

74. (canceled)

75. (canceled)
